(19) United States
(12) Patent Application Publication
(10) Pub. No.: US 2025/0032628 A1
(43) Pub. Date: Jan. 30, 2025

(54) TARGETED VESICLE COMPOSITIONS AND METHODS

(71) Applicant: CAPRICOR, INC., San Diego, CA (US)

(72) Inventors: Minghao SUN, San Diego, CA (US); Mahrou SADRI, San Diego, CA (US); Firouz MOHSENIAN, San Diego, CA (US); Kristi ELLIOTT, Encinitas, CA (US); Mafalda CACCIOTTOLO, San Diego, CA (US); Li-En HSIEH, La Jolla, CA (US); Yujia LI, San Diego, CA (US); Michael LECLAIRE, San Diego, CA (US); Meena MURALI, San Diego, CA (US)

(21) Appl. No.: 18/786,287

(22) Filed: Jul. 26, 2024

Related U.S. Application Data
(60) Provisional application No. 63/654,820, filed on May 31, 2024.
(60) Provisional application No. 63/593,197, filed on Oct. 25, 2023.
(60) Provisional application No. 63/588,301, filed on Oct. 6, 2023.
(60) Provisional application No. 63/530,299, filed on Aug. 2, 2023.
(60) Provisional application No. 63/529,116, filed on Jul. 26, 2023.

(51) Int. Cl. A61K 47/64; A61K 47/68; C07K 14/705; C07K 16/28; C12N 15/113

(52) U.S. Cl. CPC A61K 47/6425; A61K 47/6843; C07K 14/70596; C07K 16/2881; C12N 15/113; C07K 2317/569; C12N 2310/11

(57) ABSTRACT

The present disclosure relates to compositions and methods for targeting vesicles to specific tissue and cell types. Also disclosed are compositions and methods for delivering therapeutic molecules, including nucleic acids and nucleic acid derivatives, to specific cells or tissues using vesicles with cell and tissue-specific targeting moieties expressed on their surfaces.

### CROSS-REFERENCE TO RELATED APPLICATIONS

This application claims priority to U.S. Provisional Application No. 63/529,116, filed Jul. 26, 2023, U.S. Provisional Application No. 63/530,299, filed Aug. 2, 2023, U.S. Provisional Application No. 63/588,301, filed Oct. 6, 2023, U.S. Provisional Application No. 63/593,197, filed Oct. 25, 2023, and U.S. Provisional Application No. 63/654,820, filed May 31, 2024, the contents of which are incorporated herein by reference in their entireties.

### SEQUENCE LISTING

The instant application contains a Sequence Listing which has been submitted electronically in .XML format and is hereby incorporated by reference in its entirety. Said .XML copy, created on Jul. 24, 2024, is named CAP520US_SEQ_ST26.xml and is 28,380 bytes in size. The sequence listing contained in this .XML file is part of the specification and is hereby incorporated by reference herein in its entirety.

### BACKGROUND OF THE INVENTION

Many drugs and drugs in development have low safety or efficacy profiles due to problems associated with specificity, targeting, delivery to desired site-of-action, pharmacokinetics, complicated administration procedures, and patient compliance. Therefore, there exists a significant unmet need to drug delivery systems that enable lower doses, lower side effects, exquisite targeting and delivery, greater patient compliance, and ease and cost of manufacturing.

### SUMMARY OF THE INVENTION

Here, a targeted vesicle drug delivery composition and method are disclosed.

In one embodiment, an engineered vesicle that contains a fusion protein with a targeting moiety sequence and a vesicle protein sequence, such that the targeting moiety is expressed on the outer surface of the vesicle, is disclosed. In one aspect, the vesicle is an exosome.

In one aspect, the targeting moiety is a receptor ligand, a receptor, an antibody, a heavy chain only antibody (VHH), a ScFv, a virus antigen, a virus antigen receptor, or fragments or chimeras thereof.

In one aspect, the targeting moiety is a soluble protein, a type I transmembrane protein, or a type II transmembrane protein.

In one aspect, the targeting moiety binds specifically to no more than five tissues or cell-types.

In one aspect, the targeting moiety binds to muscle tissue, a muscle cell, or a muscle cell receptor.

In one aspect, the targeting moiety binds to an acetylcholine receptor, a transferrin receptor, a ryanodine receptor, a cholinergic receptor, a dystrophin, a myosin heavy chain, an alpha actinin, a PRAME family member 9, an FGF8, a protein phosphatase 1 regulatory subunit 27, an isopentenyl-diphosphate delta isomerase 2, a membrane integral NOTCH2 associated receptor 2, a SERCA2, an acetylcholine receptor epsilon, an SCN4A, a muscle specific creatine kinase (CK-MM), or a junctional sarcoplasmic reticulum protein 1.

In one aspect, the targeting moiety binds to lung tissue, a lung cell, or a lung cell receptor.

In one aspect, the targeting moiety binds to an ACE2 receptor, a surfactant protein, a secretoglobin family member, an advanced glycosylation end-product specific receptor, a membrane spanning 4-domains A15, a napsin A aspartic peptidase, a rhotekin 2, a solute carrier family 34 member, a mannose receptor C-type 1, a macrophage receptor, a mast cell expressed membrane protein, a mesothelin, or a periaxin.

In one aspect, the targeting moiety is an antibody, an ScFv, or a VHH.

In one aspect, the targeting moiety is a virus glycoprotein, such as, e.g., a coronavirus spike glycoprotein, an influenza hemagglutinin, an influenza neuraminidase, a respiratory syncytial virus F glycoprotein, or a respiratory syncytial virus G glycoprotein, or more specifically, a SARS-CoV-2 spike protein.

In one aspect, the vesicle protein is selected from the group consisting of Lamp-1, Lamp-2, CD13, Flotillin, Syntaxin −3, CD44, ICAM-1, Integrin alpha4, L1CAM, LFA-1, Vti-1A and B, CD9, CD37, CD53, CD63, CD81, CD82, CD151, ICAM-1 and tetraspanins. In a specific aspect, the vesicle protein is a CD9 protein.

In one specific aspect, the fusion protein contains, in order from amino terminus to carboxy terminus, a SARS-CoV-2 spike protein polypeptide, a linker polypeptide, and a CD9 polypeptide, as depicted, e.g., in FIGS. 1A and 1B.

In another specific aspect, the fusion protein contains, in order from amino terminus to carboxy terminus, a signal peptide, an ScFv or VHH protein polypeptide, a hinge region, a transmembrane domain polypeptide, a linker polypeptide, and a CD9 polypeptide, as depicted, e.g., in FIGS. 2A and 2B.

In one aspect, the vesicle contains a cargo molecule(s), such as for delivery to the target specified by the targeting moiety. Useful cargos include, for example, inter alia, a fluorescent dye, a hydrophobic small molecule drug, a hydrophilic small molecule drug, a nucleic acid, a peptide, a peptide amino acid, an antibody or antibody fragment, and/or a contrast agent.

In more specific aspects, the drug cargo is an antisense oligonucleotide (ASO) or a small interfering RNA (siRNA).

Disclosed is a vesicle composition, a method for making the vesicle composition, and a method for using the vesicle composition, such that the vesicle contains an exogenous or synthetic polynucleotide.

Regarding the vesicle composition, the exogenous or synthetic polynucleotide may be positioned proximate the membrane and outside of the vesicle proper, or the exogenous or synthetic polynucleotide may be positioned inside the lumen of the exosome. Both positions are included within the term “contains.”

Here, a preferred vesicle is an extracellular vesicle, preferably a secreted vesicle such as an exosome. The synthetic or exogenous polynucleotide contained within the vesicle may be an RNA, such as mRNA, siRNA, and/or miRNA, and/or a DNA, preferably an antisense oligonucleotide (ASO). The polynucleotide may be modified or may be native in structure. Useful modifications include cholesterol adducts, 2′-O-methyl phosphorothioate, phophorodithioate, methylphosphonate, and/or phosphorodiamidate modifications. Preferred modified polynucleotides include phosphorodiamidate morpholino (PMO) nucleic acids.

In one embodiment, the composition contains an exosome loaded with an ASO. Here, the ASO may be a dystrophin exon skipping antisense oligonucleotide. The ASO may be positioned outside the exosome and proximate the membrane, or within the lumen of the exosome. In those situations where the polynucleotide is positioned outside the exosome proximate the membrane, the polynucleotide may be linked to a moiety that can bind to an exosomal surface protein.

In a specific embodiment, the composition contains an exosome loaded with a dystrophin exon-skipping ASO (e.g., exon 51, exon 53, or the like), wherein the exosome has expressed on its surface a muscle-targeting moiety, such as, e.g., an anti-transferrin receptor 1 moiety, which is fused to an exosomal protein such as, e.g., CD9 or the like. The muscle-targeted anti-TfR1 expressing exosome containing the dystrophin exon-skipping ASO was observed to specifically or preferentially target and deliver the ASO to skeletal muscle relative to untargeted exosomes.

The vesicle of the composition may be derived from any cell source. Preferred cell sources for the vesicle include HEK293 cells and their derivatives, and cardiosphere-derived cells (CDCs).

Regarding the method for making the vesicle that contains the exogenous or synthetic polynucleotide, the steps described hereinafter refer to the various polynucleotide-containing vesicle embodiments described above. In one method-of-making embodiment, (i) the vesicle, as described above, is contacted with a polynucleotide, as described above, (ii) pores are opened in the vesicle membrane, thus permitting the polynucleotide to enter the lumen of the vesicle, and (iii) the pores are closed thereby containing the polynucleotide within the lumen of the vesicle.

Here, the pores can be opened in the exosome membrane by sonication, electroporation, heat shock, and/or freeze-thaw, and the pores can be closed by resting the vesicle/polynucleotide mixture on ice. For heat shocking, (i) the vesicle, Calcium chloride, and the polynucleotide are combined, (ii) the combination is placed on ice, (iii) the combination is then exposed to heat at 42° C., and then (iv) the heat exposed combination is placed on ice to produce a recovered vesicle that contains the polynucleotide. For electroporation, in some cases, nucleic acid arcing products (e.g., aggregates) may be formed, which are subsequently removed from the polynucleotide-loaded exosomes.

In an embodiment in which the synthetic or exogenous polynucleotide is positioned outside the exosome proximate the exosome membrane, (i) the polynucleotide is linked to a moiety that can bind to a vesicle protein, and (ii) the linked polynucleotide is combined with the vesicle. Here, the moiety binds to the vesicle protein thereby linking the polynucleotide to the exosome proximate to the membrane such that the polynucleotide is displayed on/proximate the surface of the exosome. In a preferred embodiment, the moiety is CP05 and the vesicle protein is CD63. In a preferred embodiment, the polynucleotide is an ASO that targets a dystrophin exon.

Regarding the method of treating a subject with the polynucleotide-loaded vesicle composition described hereinabove or the polynucleotide-loaded vesicle composition made according to the method described hereinabove (or in the alternate verbiage, the use of said composition to treat said subject), in one embodiment, the polynucleotide-loaded vesicle composition is administered to a subject wherein the polynucleotide is directed to a disease or condition of which the subject suffers. In a preferred embodiment, the subject suffers from muscular degeneration, the vesicle is an exosome from a HEK293 derivative cell, and the polynucleotide is a dystrophin exon-skipping ASO.

### BRIEF DESCRIPTION OF THE DRAWINGS

FIG. 1A depicts a linear cartoon of a SARS-CoV-2 spike CD9 fusion protein oriented from left-to-right amino-terminus-to-carboxy-terminus with a linker sequence positioned between the spike protein polypeptide and the CD9 protein polypeptide.

FIG. 1B depicts a cartoon of a SARS-CoV-2 spike CD9 fusion protein of FIG. 1A relative to a vesicle membrane oriented from left-to-right amino-terminus-to-carboxy-terminus. The spike protein polypeptide spans the membrane once with its amino terminus oriented on the outside of the vesicle. The linker is positioned on the luminal side of the membrane. The CD9 protein polypeptide spans the membrane 4 times with its carboxy terminus oriented in the lumen of the vesicle.

FIG. 2A depicts a linear cartoon of an ScFv or VHH CD9 fusion protein oriented from left-to-right amino-terminus-to-carboxy-terminus with an amino terminal signal peptide fused to an ScFv or VHH polypeptide fused to a hinge region peptide fused to a transmembrane domain peptide fused to a linker peptide fused to a CD9 protein polypeptide.

FIG. 2B depicts a cartoon of an ScFv or VHH CD9 fusion protein of FIG. 2A relative to a vesicle membrane oriented from left-to-right amino-terminus-to-carboxy-terminus. The nucleocapsid protein polypeptide with the amino terminal signal sequence is positioned on the outside (cytoplasmic or external side) with a linker sequence connecting the nucleocapsid protein polypeptide to the transmembrane domain peptide, which spans the membrane and which in turn connects to the linker positioned in the lumen and which connects to the CD9 protein polypeptide which spans the membrane 4 times with its carboxy terminus oriented in the lumen of the vesicle.

FIG. 3A is a flow chart depicting the elements and steps for producing cells expressing a spike protein polypeptide fusion protein using a packaging cell (301) and a host cell (311) mediated by a lentivirus vector (304).

FIG. 3B is a histogram depicting relative fluorescent intensity flow analysis of host cells expressing spike protein on their surface.

FIG. 4A is a graph depicting the concentration of spike-expressing exosomes per milliliter as a function of exosome diameter in nanometers.

FIG. 4B is a western blot stained for SARS-CoV-2 spike protein. The first column from left to right depicts lane loaded with size markers, second column represents lane loaded with non-transduced 293F (host cell) protein, third column represents lane loaded with protein from exosomes derived from non-transduced 293F cells, fourth column represents lane loaded with protein from 293F cells constitutively expressing spike fusion protein, fifth column represents lane loaded with protein from exosomes derived from 293F cells constitutively expressing spike fusion protein, and sixth column represents lane loaded with spike fusion protein.

FIG. 4C is a histogram depicting relative fluorescent intensity flow analysis of exosomes with spike expressed on the surface. The left curve represents exosomes derived from 293F cells that do not express a spike-CD9 fusion protein. The right curve represents exosomes derived from 293F cells expressing a spike-CD9 fusion protein.

FIG. 5A depicts a transmission electron micrograph of exosomes expressing SARS-CoV-2 spike protein. Inset is a blow up showing an exosome decorated with spike protein. Arrows point to SARS-CoV-2 spikes around the circumference of exosomes.

FIG. 5B depicts a higher magnification transmission electron micrograph of exosomes expressing SARS-CoV-2 spike protein. Arrows point to SARS-CoV-2 spikes around the circumference of exosomes.

FIG. 6 is a line graph depicting the percent uptake of exosomes by HEK293-hACE2 cells as a function of exosome concentration expressed in number of exosomes per mL. Series 1 (blue) represents HEK293-hACE2 cell-uptake of 293F exosomes that do not express spike protein. Series 2 (orange) represents HEK293-hACE2 cell-uptake of spike-expressing exosomes.

FIG. 7 is a graphical representation of an anti-TfR1 VHH CD9 fusion protein showing from amino terminus (left) to carboxy terminus (right) a first VH domain, a second VH domain, a CD8 sequence containing a transmembrane domain, and a CD9 sequence containing its four transmembrane spanning domains (tetraspannin).

FIG. 8 is a western blot probed with an anti-CD9 antibody. The first column (from left to right) is a molecular weight ladder. The second column contains non-engineered 293F cell extract. The third column contains non-engineered 293F exosomes extract. The fourth column contains CD9 knockout 293F cell extract. The fifth column contains CD9 knockout, αTfR1-VHH-CD9 engineered 293F cell extract.

FIGS. 9A-9C depict anti-CD9 flow cytometry histograms showing anti-CD9 fluorescence intensity as a function of cell count. FIG. 9A depicts non-engineered 293F cells showing endogenous CD9 expression. FIG. 9B depicts CD9 knockout 293 cells showing a lack of CD9 expression. FIG. 9C depicts CD9 knock out 293 cells expressing an αTfR1-VHH-CD9 fusion showing expression of CD9 antigen.

FIGS. 10A-10C depict flow cytometry scatter plots showing exosome fluorescence intensity as a function of cell number. FIG. 10A depicts C2C12 cells contacted with PBS. FIG. 10B depicts C2C12 cells contacted with non-engineered 293F-derived exosomes. FIG. 10C depicts C2C12 cells contacted with 293F-derived exosomes engineered to express an αTfR1 targeting moiety.

FIGS. 11A-11C depict fluorescent images of mouse tissues obtained from mice injected with (i) PBS, (ii) non-engineered 293F exosomes containing a fluorescent label, or (iii) 293F-derived exosomes engineered to express an αTfR1 targeting moiety containing a fluorescent label. FIG. 11A, FIG. 11B, and FIG. 11C are iterations of the same experiment with different mice. For example, aTFR signal was recorded into muscle in 5 out of six (83%) mice tested (last two rows, last column of each panel).

FIG. 12 depicts a compilation flow cytometry histogram showing fluorescence intensity of exosomes as a function of labeled ASO concentration. Top row (row 1) shows unloaded αTfR1 exosomes. Row 2 shows αTfR1 exosomes loaded with 500 pM ASO. Row 3 shows αTfR1 exosomes loaded with 1 nM ASO. Row 4 shows αTfR1 exosomes loaded with 2 nM ASO. Row 5 shows αTfR1 exosomes loaded with 3 nM ASO.

FIG. 13 depicts fluorescent images of mouse tissues obtained from mice injected with (i) PBS (row 1), (ii) Cy5.5-labeled antisense oligonucleotide (ASO) (row 2), or (iii) 293F-derived exosomes engineered to express an αTfR1 targeting moiety containing a Cy5-5-labeled ASO (row 4).

FIG. 14A depicts fluorescent images of mouse hindlimb muscle tissues obtained from mice injected with (i) PBS (row 1), (ii) 5 doses of 293F-derived exosomes engineered to express an αTfR1 targeting moiety containing a Cy5-5-labeled ASO (row 2), (iii) 3 doses of 293F-derived exosomes engineered to express an αTfR1 targeting moiety containing a Cy5-5-labeled ASO (row 3), and 1 dose of 293F-derived exosomes engineered to express an αTfR1 targeting moiety containing a Cy5-5-labeled ASO (row 4).

FIG. 14B is a histogram depicting Cy5-5-labeled ASO fluorescence intensity of mouse hindlimb muscle as a function of number of does of 293F-derived exosomes engineered to express an αTfR1 targeting moiety containing a Cy5-5-labeled ASO. Column 1 depicts PBS control; column 2 depicts 5 doses; column 3 depicts 3 doses; and column 4 depicts 1 dose.

FIG. 15A depicts fluorescent images of mouse heart tissues obtained from mice injected with (i) PBS (row 1), (ii) 5 doses of 293F-derived exosomes engineered to express an αTfR1 targeting moiety containing a Cy5-5-labeled ASO (row 2), (iii) 3 doses of 293F-derived exosomes engineered to express an αTfR1 targeting moiety containing a Cy5-5-labeled ASO (row 3), and 1 dose of 293F-derived exosomes engineered to express an αTfR1 targeting moiety containing a Cy5-5-labeled ASO (row 4).

FIG. 15B is a histogram depicting Cy5-5-labeled ASO fluorescence intensity of mouse heart tissue as a function of number of does of 293F-derived exosomes engineered to express an αTfR1 targeting moiety containing a Cy5-5-labeled ASO. Column 1 depicts PBS control; column 2 depicts 5 doses; column 3 depicts 3 doses; and column 4 depicts 1 dose.

FIG. 16 is nucleic acid blot depicting mouse dystrophin and exon 23-skipped mouse dystrophin nucleic acid. Column 1 depicts a nucleic acid ladder; column 2 targeted exosomes loaded with 24 uM exon 23 skipping PMO ASO showing 86% exon skipping; column 3 targeted exosomes loaded with 8 uM exon 23 skipping PMO ASO showing 44% exon skipping; column 4 targeted exosomes loaded with 2.4 uM exon 23 skipping PMO ASO showing 22% skipping; column 5 naked 24 uM exon 23 skipping PMO ASO showing 32% exon skipping; column 6 naked 8 uM exon 23 skipping PMO ASO showing 19% exon skipping; column 7 naked 2.4 uM exon 23 skipping PMO ASO showing 0% exon skipping; column 8 endoporter delivered 24 uM exon 23 skipping PMO ASO showing 71% exon skipping; column 9 endoporter delivered 8 uM exon 23 skipping PMO ASO showing 38% exon skipping; column 10 endoporter delivered 2.4 uM exon 23 skipping PMO ASO showing 0% exon skipping.

FIG. 17 is nucleic acid blot depicting mouse dystrophin and exon 23-skipped mouse dystrophin nucleic acid. Column 1 depicts a nucleic acid ladder; column 2 targeted exosomes loaded with 24 uM exon 23 skipping PMO ASO showing 86% exon skipping; column 3 targeted exosomes loaded with 8 uM exon 23 skipping PMO ASO showing 44% exon skipping; column 4 targeted exosomes loaded with 2.4 uM exon 23 skipping PMO ASO showing 22% skipping; column 5 naked 24 uM exon 23 skipping PMO ASO showing 32% exon skipping; column 6 naked 8 uM exon 23 skipping PMO ASO showing 19% exon skipping; column 7 naked 2.4 uM exon 23 skipping PMO ASO showing 0% exon skipping; column 8 endoporter delivered 24 uM exon 23 skipping PMO ASO showing 71% exon skipping; column 9 endoporter delivered 8 uM exon 23 skipping PMO ASO showing 38% exon skipping; column 10 endoporter delivered 2.4 uM exon 23 skipping PMO ASO showing 0% exon skipping.

FIG. 18 is a histogram depicting percent Cy3 expression flow in a cell population as a function of filtration procedure and construct. From left to right, bar 1 represents 293F exosomes control, bar 2 represents exosomes plus RRM2 control, bar 3 represents electroporated (EP) RRM3 Cy3 siRNA control, bar 4 represents exosomes loaded with RRM2-Cy3 siRNA followed by filtration method F01, bar 5 represents electroporated (EP) RRM2 followed by filtration method F01, bar 6 represents RRM3 Cy3 siRNA only followed by filtration method F01, bar 7 represents exosomes loaded with RRM2-Cy3 siRNA followed by filtration method F02, bar 8 represents electroporated (EP) RRM2 followed by filtration method F02, bar 9 represents RRM3 Cy3 siRNA only followed by filtration method F02, bar 10 represents exosomes loaded with RRM2-Cy3 siRNA followed by filtration method F03, bar 11 represents electroporated (EP) RRM2 followed by filtration method F03, bar 12 represents RRM3 Cy3 siRNA only followed by filtration method F03.

FIG. 19 is a histogram depicting RRM2 siRNA in nanograms per microliter recovered as a function of filtration process and construct. From left to right, bar 1 represents spike-in control, bar 2 represents exosomes loaded with RRM2-Cy3 siRNA followed by filtration method F01, bar 3 represents electroporated (EP) RRM2 followed by filtration method F01, bar 4 represents RRM3 Cy3 siRNA only followed by filtration method F01, bar 5 represents exosomes loaded with RRM2-Cy3 siRNA followed by filtration method F02, bar 6 represents electroporated (EP) RRM2 followed by filtration method F02, bar 7 represents RRM3 Cy3 siRNA only followed by filtration method F02, bar 8 represents exosomes loaded with RRM2-Cy3 siRNA followed by filtration method F03, bar 9 represents electroporated (EP) RRM2 followed by filtration method F03, bar 10 represents RRM3 Cy3 siRNA only followed by filtration method F03.

FIG. 20 is a histogram depicting quantity of siRNA delivered to cells by loaded exosomes as a function of filtration process and construct. From left to right, bar 1 represents spike-in control, bar 2 represents exosomes loaded with RRM2-Cy3 siRNA followed by filtration method F01, bar 3 represents electroporated (EP) RRM2 followed by filtration method F01, bar 4 represents RRM3 Cy3 siRNA only followed by filtration method F01, bar 5 represents exosomes loaded with RRM2-Cy3 siRNA followed by filtration method F02, bar 6 represents electroporated (EP) RRM2 followed by filtration method F02, bar 7 represents RRM3 Cy3 siRNA only followed by filtration method F02, bar 8 represents exosomes loaded with RRM2-Cy3 siRNA followed by filtration method F03, bar 9 represents electroporated (EP) RRM2 followed by filtration method F03, bar 10 represents RRM3 Cy3 siRNA only followed by filtration method F03.

FIG. 21 is a histogram depicting percent exosomes loaded Cy3-labeled RRM2 siRNA as a function of filtration method and construct for two dated experiments. The histogram bars are presented two per experiment, with the first bar of the pair representing experiments of day-1 and the second bar of the pair representing experiments two days later. From left to right, bar pair 1 represents spike-in control, bar pair 2 represents exosomes loaded with RRM2-Cy3 siRNA followed by filtration method F01, bar pair 3 represents electroporated (EP) RRM2 followed by filtration method F01, bar pair 4 represents RRM3 Cy3 siRNA only followed by filtration method F01, bar pair 5 represents exosomes loaded with RRM2-Cy3 siRNA followed by filtration method F02, bar pair 6 represents electroporated (EP) RRM2 followed by filtration method F02, bar pair 7 represents RRM3 Cy3 siRNA only followed by filtration method F02, bar pair 8 represents exosomes loaded with RRM2-Cy3 siRNA followed by filtration method F03, bar pair 9 represents electroporated (EP) RRM2 followed by filtration method F03, bar pair 10 represents RRM3 Cy3 siRNA only followed by filtration method F03, bar pair 11 represents exosomes loaded with RRM2-Cy3 siRNA followed by filtration method F04, bar pair 12 represents electroporated (EP) RRM2 followed by filtration method F04, bar pair 13 represents RRM3 Cy3 siRNA only followed by filtration method F04.

FIG. 22 is a histogram depicting quantity of siRNA delivered to SKOV2 cells by loaded exosomes and subsequent gene knock-down as a function of filtration process and construct. From left to right, bar 1 represents spike-in control, bar 2 represents exosomes loaded with RRM2-Cy3 siRNA followed by filtration method F01, bar 3 represents electroporated (EP) RRM2 followed by filtration method F01, bar 4 represents RRM3 Cy3 siRNA only followed by filtration method F01, bar 5 represents exosomes loaded with RRM2-Cy3 siRNA followed by filtration method F02, bar 6 represents electroporated (EP) RRM2 followed by filtration method F02, bar 7 represents RRM3 Cy3 siRNA only followed by filtration method F02, bar 8 represents exosomes loaded with RRM2-Cy3 siRNA followed by filtration method F03, bar 9 represents electroporated (EP) RRM2 followed by filtration method F03, bar 10 represents RRM3 Cy3 siRNA only followed by filtration method F03, bar 11 represents exosomes loaded with RRM2-Cy3 siRNA followed by filtration method F04, bar 12 represents electroporated (EP) RRM2 followed by filtration method F04, bar 13 represents RRM3 Cy3 siRNA only followed by filtration method F04.

FIG. 23 is a histogram depicting quantity of FAM-labeled luciferase antisense oligonucleotide (f-Luc-ASO-FAM) as a function of filtration process and construct. From left to right, bar 1 represents 293F exosomes control, bar 2 represents exosomes plus f-Luc-ASO-FAM control, bar 3 represents electroporated (EP) f-Luc-ASO-FAM control, bar 4 represents exosomes loaded with f-Luc-ASO-FAM followed by filtration method F01, bar 5 represents electroporated (EP) f-Luc-ASO-FAM followed by filtration method F01, bar 6 represents f-Luc-ASO-FAM only followed by filtration method F01, bar 7 represents exosomes loaded with f-Luc-ASO-FAM followed by filtration method F02, bar 8 represents electroporated (EP) f-Luc-ASO-FAM followed by filtration method F02, bar 9 represents f-Luc-ASO-FAM only followed by filtration method F02, bar 10 represents exosomes loaded with f-Luc-ASO-FAM followed by filtration method F03, bar 11 represents electroporated (EP) f-Luc-ASO-FAM followed by filtration method F03, bar 12 represents f-Luc-ASO-FAM only followed by filtration method F03.

FIG. 24A is a histogram representing total loaded ARG1 mRNA in nanograms in exosomes as a function of method of loading exosomes. Bar 1 represents unloaded vehicle exosomes, bar 2 represents ARG1 mRNA loaded via calcium chloride heat shock, bar 3 represents ARG1 mRNA loaded via freeze-thaw.

FIG. 24B is a histogram representing ARG1 mRNA percent loading efficiency into exosomes as a function of loading method. Bar 1 represents loading via calcium chloride heat shock, bar 2 represents loading via freeze-thaw.

FIGS. 25A-25D are histograms depicting relative ARG1 mRNA expression (fold change in expression) in cells as a function of input treatment and inputs. FIG. 25A depicts from left to right untreated HEK cells and HEK cells treated with ARG1 loaded exosomes. FIG. 25B depicts from left to right HEK cells treated with unloaded free exosomes and HEK cells treated with ARG1 loaded exosomes. FIG. 25C depicts from left to right HEK cells treated with RNase-treated exosomes and HEK cells treated with ARG1 loaded exosomes. FIG. 25D depicts from left to right HEK cells treated with ARG1 loaded exosomes further treated with RNase and HEK cells treated with ARG1 loaded exosomes.

FIG. 26 is a histogram depicting ARG1 mRNA expression in HEK293 cells as a function of mRNA delivery method. From left to right, bar 1 depicts naked mRNA, bar 2 depicts exosome-based ARG1 mRNA delivery, bar 3 depicts lipofectamine-based delivery of ARG1 mRNA.

FIG. 27 is a bioanalyzer blot depicting ARG1 mRNA. Lanes from left to right, lane 1 ladder, lane 2 10 ng ARG mRNA, lane 3 10 ng ARG mRNA, lane 4 5 ng ARG mRNA, lane 5 5 ng ARG mRNA, lane 6 1 ng ARG mRNA, lane 7 1 ng ARG mRNA, lane 8 exosomes loaded with ARG mRNA, lane 9 exosomes loaded with ARG mRNA, lane 10 exosomes without AGR mRNA, lane 11 exosomes without AGR mRNA, lane 12 water, lane 13 water.

FIG. 28A is a histogram depicting percent siRNA loading efficiency into exosomes as a function of loading method, from left to right, calcium chloride heat shock and freeze thaw methods. FIG. 28B is a histogram depicting total siRNA loaded into exosomes as a function of loading, from left to right, unloaded vehicle exosomes, calcium chloride heat shock loading, and freeze thaw loading.

FIGS. 29A-29C are histograms depicting relative luciferase mRNA expression as fold change as a function of exosome delivery of luciferase siRNA. FIG. 29A depicts relative luciferase mRNA expression at 24 hours with 3.2E11 exosomes containing 10 micrograms luciferase siRNA (second bar). FIG. 29B depicts expression at 48 hours with 3.2E11 exosomes containing 10 micrograms luciferase siRNA (second bar). FIG. 29C depicts expression at 48 hours with 3.2E11 exosomes containing 10 micrograms luciferase siRNA (second bar) and 6.4E11 exosomes containing 20 micrograms luciferase siRNA (third bar).

FIG. 30A is a histogram depicting the percentage of murine dystrophin exon 23 exon skipping as a function of drug delivered to C2C12 cells. The X-axis represents 6 experiment samples with three bars per experiment sample. From left to right, the first bar represents naked exon 23 PMO ASO, the second bar represents CP05-conjugated exon 23 PMO ASO, the third bar represents exosomes containing exon 23 PMO ASO.

FIG. 30B is a histogram depicting abundance of exon 23 skipped dystrophin relative to actin as a function of cell type and exon 23 PMO ASO treatment.

FIG. 31 is a flow diagram depicting fluorescein signal intensity as a function of the concentration of FITC-labeled ASO loaded into exosomes. Here, anti-TFR1 expressing exosomes were loaded with increasing amounts of FAM labelled dystrophin exon-skipping ASO and analyzed by flow cytometry. Shown is fluorescence intensity as a function of quantity of loaded anti-sense oligonucleotide. Here, the exosomes were loaded using a CaCl heat shock method.

FIG. 32A is a photograph of far-red-excited test tubes. The first tube contains PBS. The second tube contains unlabeled or unloaded exosomes, the third tube contains Cy5-labeled ASO. The fourth tube contains Cy5-ASO-loaded exosomes.

FIG. 32B is a photograph of tissues from mice injected with Tfr1-targeted HEF293 exosomes loaded with Cy5-labeled ASO. Row 1 represents PBS injected controls (tissues from left to right are salivary gland, lungs, spleen, diaphragm, and lower limbs). Row 2 represents ASO alone injected (tissues from left to right are salivary gland, lungs, spleen, and lower limbs). Rows 3-5 represent Cy5-ASO-loaded anti-TfR1 exosomes (tissues from left to right are salivary gland, lungs, spleen, and lower limbs). Here, exosomes carrying an anti-TfR targeting moiety were loaded with Cy5.5 labeled exon-skipping ASO and injected into wild-type Balb/c female mice by I.V., the tissue was imaged at 24 hours post I.V. injection. Tissue shown are lower limb skeletal muscle. Here, 2 of 5 mice showed a good accumulation of labeled signal in the lower limbs in the first try but not observed in the ASO only injected mice.

FIG. 33 is a FACS plot of HEK293F derived exosomes loaded with Cy5-labeled ASO using the CaCl2) heat shock method.

FIGS. 34A-34D are fluorescence micrographs of HEK293 cells treated/contacted with different reagents. FIG. 34A depicts cells contacts with Cy5-labeled ASO-loaded exosomes. FIG. 34B depicts cells contacted with Cy5-labeled ASO formulated with lipofectamine. FIG. 34C depicts cells contacted with ASO alone. FIG. 34D depicts cells contacts with unloaded exosomes.

FIGS. 35A and 35B are FACS dot plots of exosomes loaded with PE-labeled luciferase siRNA. FIG. 35A depicts unloaded exosomes being positive for CD9 and negative for fLuc-siRNA (quadrant 3, 94.9%). FIG. 35B depicts fLuc-siRNA-loaded exosomes showing a clear population of loaded exosomes positive for both APC CD9 (exosome marker) and PE siRNA (quadrant 4, 98.6%).

FIG. 36 is a histogram depicting relative luciferase mRNA expression in cells (fold change) as a function of treatment, from left to right, naked exosomes, luciferase siRNA loaded exosomes, and luciferase siRNA.

FIG. 37 is a citation assay histogram depicting lissamine signal intensity as a function of exosome loading material, from left to right, naked exosomes, naked PMO, and exosome loaded lissamine-labeled PMO.

FIG. 38 is a FACS histogram of exosomes loaded with varying amounts (5 micromoles to 10 micromoles) of PE-labeled PMO.

FIG. 39 is a FACS histogram of cells transfected with various PMO constructs including naked exosomes, oligo PMO, naked PMO, and PMO-containing exosomes

FIG. 40 is a FACS histogram of exosomes loaded with various concentrations of PMO ASO exon 51 skipper (5M, 10 uM, and 20 uM) using the disclosed heat shock method and with one filtration step.

FIG. 41 is a FACS dot plot of exosomes loaded with labeled siRNA.

FIG. 42 is a FACS dot plot of exosomes loaded with labeled siRNA.

FIG. 43 is a histogram depicting exosome loading efficiency as a function of input, from left to right, loaded exosomes, electroporated siRNA, siRNA only, and QIAZOL spike-in.

FIGS. 44A-44D are photomicrographs of SKOV-3 cells stained with Hoechst and propidium iodide. FIG. 44A depicts untreated cells. FIG. 44B depicts cells treated with RRM2 Cy3-siRNA-loaded exosomes. FIG. 44C depicts cells treated with electroporated RRM2 siRNA. FIG. 44D depicts cells treated with siRNA alone.

FIG. 45, panels A and B, are histograms depicting percent SKOV-3 cell viability (Panel A) and relative RRM2 gene expression (Panel B) as a function of treatment, from left to right, untreated, siRNA-loaded exosomes, electroporated siRNA, and siRNA only.

FIG. 46 is a histogram depicting relative expression of RRM2 protein in SKOV-3 cells as determined by western blot as a function of treatment, from left to right, untreated, exosomes loaded with Cy3-siRNA-RRM2, electroporated siRNA, and siRNA only.

FIG. 47A is a histogram depicting caspace3/7 fluorescence in cells as a function of treatment, from left to right, untreated, exosomes loaded with Cy3-siRNA-RRM2, electroporated siRNA, and siRNA only.

FIG. 47B is a histogram depicting fold induction of apoptosis in cells as a function of treatment, from left to right, untreated, exosomes loaded with Cy3-siRNA-RRM2, electroporated siRNA, and siRNA only.

FIG. 48 is a cartoon depicting exosomes expressing a targeting moiety (e.g., anti-TfR1) that engages with its cognate target protein on muscle cells to effectuate delivery of the exosome cargo to muscle cells.

FIG. 49 is a photograph of tissues from mice injected with Tfr1-targeted HEF293 exosomes loaded with Cy5-labeled ASO. Tissues from left to right are salivary gland, brain, lungs, heart, liver [blocked due to high expected signal], spleen, kidney [blocked due to high expected signal], and lower limb skeletal muscle). Rows 1 and 2 represent PBS injected controls. Rows 3 and 4 represent wildtype exosome injection (no targeting). Rows 5 and 6 represent anti-TfR1 exosomes (targeted exosomes).

### DETAILED DESCRIPTION OF EMBODIMENTS

### Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “and/or” refers to and encompasses any and all possible combinations of one or more of the associated listed items, as well as the lack of combinations when interpreted in the alternative (or).

The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of ±up to 20 amino acid residues, ±up to 15 amino acid residues, ±up to 10 amino acid residues, ±up to 5 amino acid residues, ±up to 4 amino acid residues, ±up to 3 amino acid residues, ±up to 2 amino acid residues, or even ±1 amino acid residue.

The term “derived from” as in “A is derived from B” means that A is obtained from B in such a manner that A is not identical to B.

The terms “treat”, “therapeutic”, “prophylactic” and “prevent” are not intended to be absolute terms. Treatment, prevention and prophylaxis can refer to any delay in onset, amelioration of symptoms, improvement in patient survival, increase in survival time or rate, etc. Treatment, prevention, and prophylaxis can be complete or partial. The term “prophylactic” means not only “prevent”, but also minimize illness and disease. For example, a “prophylactic” agent can be administered to a subject, e.g., a human subject, to prevent infection, or to minimize the extent of illness and disease caused by such infection. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

A treatment can be considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. One skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder, and relates to a sufficient amount of therapeutic composition to provide the desired effect. The term “therapeutically effective amount” refers to an amount of a composition or therapeutic agent that is sufficient to provide a particular effect when administered to a typical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, the term “pharmaceutically acceptable” refers to a carrier that is compatible with the other ingredients of the formulation and not deleterious to the recipient thereof. The term is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “dose” and “dosage” are used interchangeably herein. A dose refers to the amount of active ingredient given to an individual at each administration. For the present invention, the dose can refer to the concentration of the extracellular vesicles or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The phrase “at least one” includes “a plurality”.

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10:0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

The term “native form” corresponds to the polypeptide as it is understood to be encoded by the infectious agent's genome. The term “exosomal form” corresponds to any derivative of the protein that, in whole or in part, is fused to an exosome-associated protein. The term “cytoplasmic form” corresponds to any derivative of the protein that, in whole or in part, is configured, or designed, to be expressed within the cytoplasm of the cell, rather than entering the canonical secretory pathway.

The expression that a certain protein is “configured, or designed, to be expressed” in a certain way means that its nucleotide sequence encodes certain a particular amino acid sequence such that when that protein is expressed in a cell, that protein will be in its native form, exosomal form, or cytoplasmic form by virtue of that particular amino acid sequence. For instance, if a spike protein(S) is expressed in its native form, it is configured, or designed, to induce a humoral or cellular immune response by virtue of the fact that it is a transmembrane protein with an extracellular domain.

The term “extracellular vesicle” (EV) refers to lipid bilayer-delimited particles that are naturally released from cells. EVs range in diameter from around 20-30 nanometers to about 10 microns or more. EVs can comprise proteins, nucleic acids, lipids and metabolites from the cells that produced them. EVs include exosomes (about 50 to about 200 nm), microvesicles (about 100 to about 300 nm), ectosomes (about 50 to about 1000 nm), apoptotic bodies (about 50 to about 5000 nm) and lipid-protein aggregates of the same dimensions.

The term “nucleic acid” refers to polynucleotides such as deoxyribonucleic acid (DNA) or ribonucleic acid (RNA), as well as chemically modified nucleic acids such as morpholino (PMO), peptide nucleic acid (PNA), 2′O-methyl, 2′ methoxy-ethyl, phosphoramidate, methylphosphonate, and phosphorothioate. Nucleic acids may be of any size. Nucleic acids include but are not limited to genomic DNA, cDNA, RNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid might be employed for introduction into, e.g., transfection of, cells, e.g., in the form of RNA which can be prepared by in vitro transcription from a DNA template. The RNA can moreover be modified before application by stabilizing sequences, capping, and polyadenylation. Generally, nucleic acid can be extracted, isolated, amplified, or analyzed by a variety of techniques such as those described by, e.g., Green and Sambrook, Molecular Cloning: A Laboratory Manual (Fourth Edition), Cold Spring Harbor Laboratory Press, Woodbury, NY 2,028 pages (2012).

“Polynucleotide” means any nucleic acid polymer, including natural polymers, polymers isolated from natural or engineered biological material (e.g., cells), and synthetic polymers. Polynucleotides include inter alia RNA, such as siRNA, mRNA, snRNA, tRNA, miRNA, rRNA, snoRNA, RNA with hairpin structure, and the like, and DNA, such as cDNA, genomic DNA and fragments of genomic DNA, oligonucleotides, single stranded oligonucleotides, anti-sense oligonucleotides (ASO), and the like, and combinations of RNA and DNA, DNA and polypeptides (e.g., aptamers), RNA and polypeptides, and the like.

“Synthetic polynucleotide” means any nucleic acid polymer made by recombinant technology or synthetic chemistry or the like. For purposes of clarity, synthetic polynucleotide is not naturally occurring nucleic acid sequence obtained from a non-engineered cell or other natural source. Synthetic polynucleotide includes DNA, RNA, and polymers with modified nucleotides/nucleosides, modified backbones, chemical modifications to either the nucleobase, the ribofuranose unit or the phosphate backbone, or other modifications, such as, e.g., 2′-O-methyl phosphorothioate, phophorodithioate, methylphosphonate, and phosphorodiamidate modifications, such as phosphorodiamidate morpholino oligomers (PMO). The term “exogenous polynucleotide” refers to a nucleic acid polymer that is positioned in a non-natural location, such as, for example, a polynucleotide that does not naturally occur in a vesicle (such as an exosome) and is positioned artificially in said vesicle.

The terms “peptide”, “polypeptide” and “protein” are used interchangeably herein and refer to any chain of at least two amino acids, linked by a covalent chemical bound. As used herein a peptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the phrase “protein polypeptide” means a polypeptide sequence of or derived from a protein. For example, a CD9 protein polypeptide may be any polypeptide of the CD9 protein, such as, e.g., a full length CD9 protein, a transmembrane domain polypeptide of a CD9 protein, a C-terminal stretch of a CD9 protein, an extracellular loop region of a CD9 protein, the intracellular (intralumenal) loop region of a CD9 protein, a C-terminal stretch of a CD9 protein, combinations thereof, and/or the like. Here, a protein polypeptide may be at least 10 amino acids long.

As used herein, the term “spike protein” includes any SARS-CoV-2 spike glycoprotein, fragment of a SARS-CoV-2 spike glycoprotein, monomer of a SARS-CoV-2 spike glycoprotein, trimer of SARS-CoV-2 spike glycoprotein monomers, variant of a SARS-CoV-2 spike glycoprotein, fusion protein or chimeral protein containing a SARS-CoV-2 spike glycoprotein sequence and another non-SARS-CoV-2 spike glycoprotein sequence, SARS-CoV-2 spike glycoproteins having one or more deletions, additions, or substitutions of one or more amino acids, and conservatively substituted variations of a SARS-CoV-2 spike glycoprotein having at least 80% amino acid sequence identity of e.g., at least the stem region of an S2 subunit, membrane-proximal stem helix region, or the receptor binding domain, or other like domains.

A fragment of a SARS-CoV-2 spike glycoprotein includes peptide or polypeptides the encompass, comprise, consist of, or overlap with e.g., antigenic epitopes, specific domains like receptor binding domain (RBD) in up or down conformational states, a receptor-binding fragment S1, fusion fragment S2, N-terminal domain (NTD), receptor-binding domain (RBD) C-terminal domain 1 (CTD1), C-terminal domain 2 (CTD2), fusion peptide (FP), fusion-peptide proximal region (FPPR), heptad repeat 1 (HR1), central helix (CH), connector domain (CD), heptad repeat 2 (HR2), transmembrane segment (TM), the cytoplasmic tail (CT), and the like.

A variant of a SARS-CoV-2 spike glycoprotein includes any known or yet to be discovered, including alpha, beta, gamma, delta, epsilon, eta, iota, kappa, 1.617.3, mu, zeta, omicron, or their subvariants, lineages, and conservatively substituted spike protein sequence.

SARS-CoV-2 spike glycoprotein may have additions, deletions, substitution, point mutations. For example, a spike protein may have a deletion of several (2-20) amino acids from its C-terminus (see Johnson et al., 2020 and Xiong et al., 2020), or furin cleavage site change (see e.g., Johnson et al., 2020).

As used herein, the term “tetraspanin” or “tetraspanin protein” means any member (or chimera thereof) of a family of proteins having four transmembrane domains and in some cases present in exosome membranes. Tetraspanin proteins are known to regulate trafficking and cell and membrane compartmentalization. Tetraspanins include inter alia CD9, CD37, CD63, CD81, CD82, CD151, TSPAN7, TSPAN8, TSPAN12, TSPAN33, peripherin, UP1a/1b, TSP-15, TSP-12, TSP3A, TSP86D, TSP26D, TSP-2, and analogs, orthologs, and homologs thereof. It is envisioned that useful tetraspanins may include chimeras of any one or more canonical tetraspanins, such as, e.g., a CD9/CD81 chimera or the like. Tetraspanins are generally described in Charrin et al., “Tetraspanins at a glance,” J Cell Sci (2014) 127 (17): 3641-3648; Kummer et al., “Tetraspanins: integrating cell surface receptors to functional microdomains in homeostasis and disease,” Med Microbiol Immunol. 2020; 209 (4): 397-405; and references cited therein. The CD9 member of the tetraspanin superfamily is generally described in Umeda et al., “Structural insights into tetraspanin CD9 function,” Nature Communications volume 11, Article number: 1606 (2020), and references cited therein.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical aIgorithm. A preferred, non-limiting example of a mathematical aIgorithm utilized for the comparison of two sequences is the aIgorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an aIgorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website).

Another preferred, non-limiting example of a mathematical aIgorithm utilized for the comparison of sequences is the aIgorithm of Myers and Miller, 1988, CABIOS 4:11-17. Such an aIgorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

For antibodies, percentage sequence identities can be determined when antibody sequences are maximally aligned by IMGT. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, multiplied by 100 to convert to percentage.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y,
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

As used herein the term “antibody” refers to immunoglobulin (Ig) molecules and immunologically active portions of immunoglobulin molecules, i.e., molecules that contain an antigen-binding site that specifically binds an antigen. Antibodies are usually heterotetrameric glycoproteins of about 150,000 Daltons, composed of two identical light (L) chains and two identical heavy (H) chains. The light chains from any vertebrate species can be assigned to one of two clearly distinct types, called kappa (κ) and lambda (A), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known. The antibody may have one or more effector functions which refer to those biological activities attributable to the Fc region (a native sequence Fc region or amino acid sequence variant Fc region or any other modified Fc region) of an antibody. Non-limiting examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells).

The term “neutralizing antibody” (Nab) refers an antibody that defends a cell from a pathogen or infectious particle by neutralizing any effect it has biologically. Neutralization renders the particle no longer infectious or pathogenic. Neutralizing antibodies are part of the humoral response of the adaptive immune system against viruses, intracellular bacteria and microbial toxin. By binding specifically to surface antigen on an infectious particle, neutralizing antibodies prevent the particle from interacting with its host cells it might infect and destroy.

Immunity due to neutralizing antibodies is also known as sterilizing immunity, as the immune system eliminates the infectious particle before any infection took place.

The term “antigen” refers to any substance that will elicit an immune response. For instance, an antigen relates to any substance, preferably a peptide or protein, that reacts specifically with antibodies or T-lymphocytes (T cells). As used herein, the term “antigen” comprises any molecule which comprises at least one epitope. For instance, an antigen is a molecule which, optionally after processing, induces an immune reaction. For instance, any suitable antigen may be used, which is a candidate for an immune reaction, wherein the immune reaction may be a cellular immune reaction. For instance, the antigen may be presented by a cell, which results in an immune reaction against the antigen. For example, an antigen is a product which corresponds to or is derived from a naturally occurring antigen. Such antigens include, but are not limited to, SARS-CoV-2 structural proteins S, N, M, and E, and any variants or mutants thereof.

The term “pharmaceutical composition” refers to a formulation comprising an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel.

Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

“Vesicle” means any lipid bound object, wherein a lipid membrane delineates an interior aqueous lumen from an external aqueous environment. The membrane may be a double membrane or a dingle membrane and may contain phospholipids, sterols, protein, and the like. Vesicles may be synthetic or natural. Natural vesicles include cell disruption debris, apoptotic bodies, secreted microvesicles, exosomes, microsomes, and the like. Vesicles can be secreted vesicles, such as extracellular vesicles (EV), including larger microvesicles, apoptotic vesicles, and smaller exosmes. Extracellular vesicles are generally described in Raposo and Stoorvogel, “Extracellular vesicles: Exosomes, microvesicles, and friends,” J. Cell Biol. Vol. 200 No. 4; 373-383; 2013.

“Exosome” means a vesicle of about 40 to 160 nm in diameter (average diameter of about 100 nm) that is secreted from a cell (e.g., mesenchymal stem cell, cardiosphere-derived cell, HEK293 cell or derivative thereof, primary cells, immortalized cell, banked cell, CHO cell, commercially available cell, insect cell, and the like). Exosomes are generally described in Kalluri and LeBleu, “The biology, function, and biomedical applications of exosomes,” Science 367, 640 (7 Feb. 2020). As used herein, an exosome may be natural or engineered. Engineered exosomes may contain or express various molecules or materials, such as, e.g., peptides and proteins, antigens, nucleic acids, biological drugs, small molecule drugs, and the like as cargo for delivery to target cells, and/or surface moieties designed to target the exosomes to specific cells or tissues, such as, e.g., muscle-targeting moieties, brain-targeting moieties, cancer cell-targeting moieties, immune cell-targeting moieties, and the like.

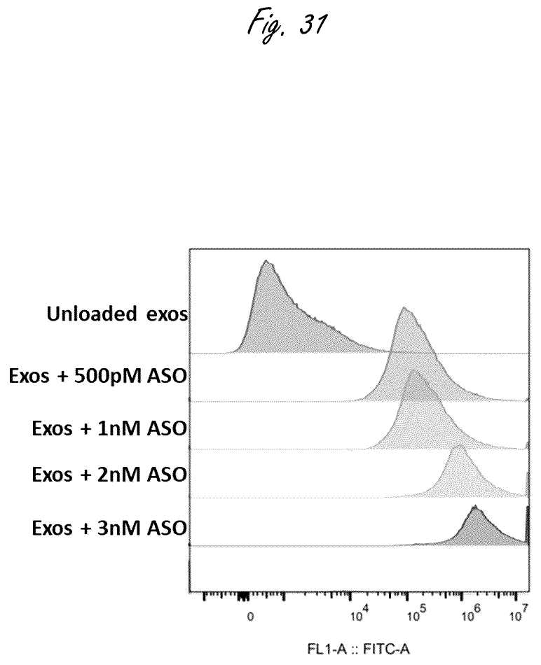

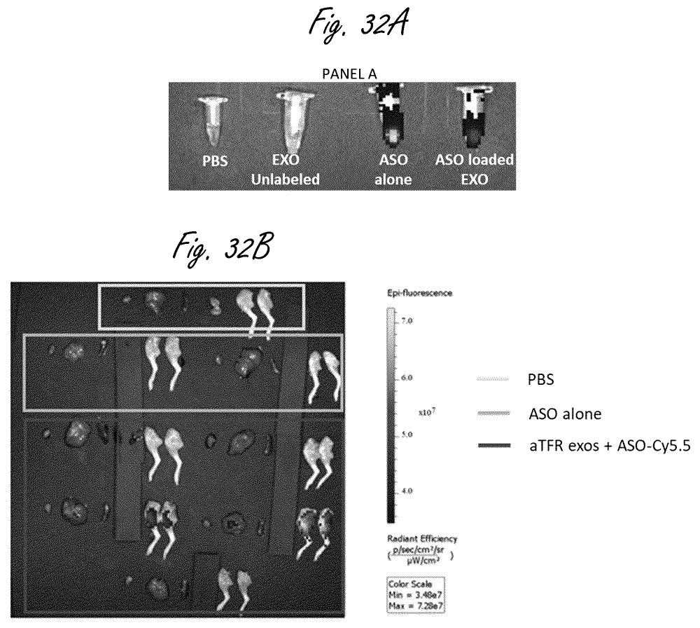

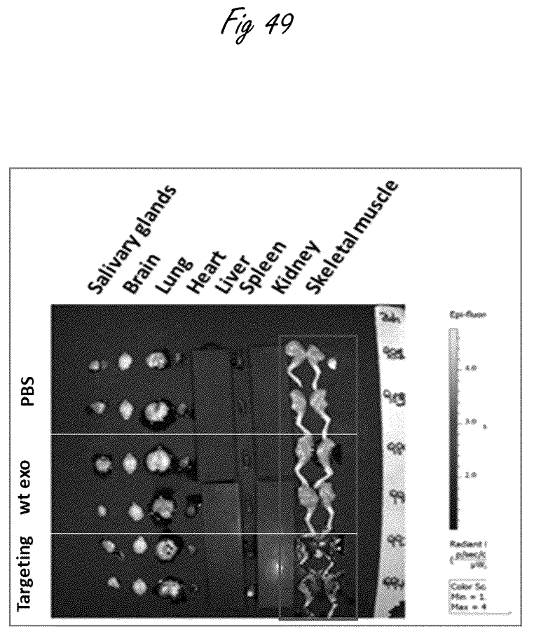

For example, engineered exosomes expressing tetraspanin or other exosomal membrane proteins fused to (covalently linked to, i.e. expressed as a fusion protein in the exosome-producing cell line) a VHH, ScFv, and/or complementarity determining region or regions that bind to transferrin receptor 1 (TfR) were produced from cells and loaded with a dystrophin exon-skipping antisense oligonucleotide (ASO) according to the method described herein (e.g., anti-TfR1-CD9 expressing 293-derived exosome loaded with exon 53 or exon 51-skipping antisense oligonucleotide, either labeled with a trackable fluorophore or unlabeled). See FIG. 31. These engineered exosomes were intravenously delivered to mice. The anti-TfR served as a muscle-targeting moiety and the engineered exosomes were observed to preferentially deliver the ASO to the muscles of the subject mice, most apparently to the muscles of the lower limb. See FIG. 32 and FIG. 49.

The term “transferrin receptor” refers to any molecule that binds to and/or directly or indirectly facilitates the movement of an iron-binding molecule across a membrane. Iron-binding molecules include but are not limited to transferrin and its various homologues and orthologues. Transferrin receptors include but are not limited to transferrin receptor 1 (TfR1), transferrin receptor 2 (TfR2), soluble transferrin receptor, and GAPDH, and their respective homologues and orthologues.

The terms “transferrin receptor binder, “transferrin receptor binding moiety,” and “transferrin receptor moiety” may be used interchangeably and refer to any compound or molecule that binds to a transferrin receptor. Such transferrin receptor binding moiety may contain, include, mimic, in whole or in part an antibody, antibody fragment, VHH, ScFv, nanobody or the like that binds a transferrin receptor, a transferrin, a transferrin fragment, or the like that binds to transferrin receptor, and fusion protein or chimeras containing same.

### Extracellular Vesicles and Exosomes

A variety of host cells are known in the art and suitable for proteins expression and extracellular vesicles production. Non-limiting examples of typical cell used for transfection include, but are not limited to, a bacterial cell, a eukaryotic cell, a yeast cell, an insect cell, or a plant cell. For example, human embryonic kidney 293 (HEK293), E. coli, Bacillus, Streptomyces, Pichia pastoris, Salmonella typhimurium, Drosophila S2, Spodoptera SJ9, CHO, COS (e.g. COS-7), 3T3-F442A, HeLa, HUVEC, HUAEC, NIH 3T3, Jurkat, 293, 293H, or 293F. See, e.g., Portolano et al., “Recombinant Protein Expression for Structural Biology in HEK 293F Suspension Cells: A Novel and Accessible Approach,” Journal of Visualized Experiments, October 2014, 92, e51897, pp. 1-8 for a description of the recombinant proteins in 293 cells in suspension culture.

Extracellular vesicles (EVs) are lipid bound vesicles secreted by cells into the extracellular space. The three main subtypes of EVs are microvesicles (MVs), exosomes, and apoptotic bodies, which are differentiated based upon their biogenesis, release pathways, size, content, and function. For a review of extracellular vesicles, see, e.g., Doyle and Wang, “Overview of Extracellular Vesicles, Their Origin, Composition, Purpose, and Methods for Exosome Isolation and Analysis,” Cells, v.8 (7), 2019 Jul., and references therein.

Exosomes include small, secreted vesicles of about 20-200 nm in diameter that are released by inter alia mammalian cells, and made either by budding into endosomes or by budding from the plasma membrane of a cell. In some cases, exosomes have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as, but not limited to, proteins, DNA and RNA (e.g., microRNA [miR] and noncoding RNA). In some embodiments, exosomes can be obtained from cells obtained from a source that is allogeneic, autologous, xenogeneic, or syngeneic with respect to the recipient of the exosomes.

Certain types of RNA, e.g., microRNA (miRNA), are known to be carried by exosomes. miRNAs function as post-transcriptional regulators, often through binding to complementary sequences on target messenger RNA transcripts (mRNAs), thereby resulting in translational repression, target mRNA degradation and/or gene silencing.

Useful exosomes can be obtained from any cell source, including prokaryotes, plants, fungi, metazoans, vertebrate, mammalian, primate, human, autologous cells and allogeneic cells. See, e.g., Kim et al., “Platform technologies and human cell lines for the production of therapeutic exosomes,” Extracell Vesicles Circ Nucleic Acids 2021; 2:3-17. For example, exosomes may be derived from mesenchymal stem cells, embryonic stem cells, iPS cells, immune cells, PBMCs, neural stem cells, HEK293 cells, which are described in e.g., Dumont et al., “Human cell lines for biopharmaceutical manufacturing: history, status, and future perspectives,” Crit Rev Biotechnol 2016; 36:1110-22, HEK293T cells, which are described in e.g., Li et al., “Identification and characterization of 293T cell-derived exosomes by profiling the protein, mRNA and MicroRNA components,” PLOS One 2016; 11: e0163043, 293F cells, Stenkamp et al., “Exosomes represent a novel mechanism of regulatory T cell suppression (P1079),” J Immunol May 1, 2013, 190 (1 Supplement) 121.11, amniotic cells, CAR-T cells, cardiospheres and cardiosphere-derived cells (CDCs), which are described in, e.g., WO2014028493, WO2022006178A1, US20210032598A1, U.S. Pat. No. 9,828,603B2, EP2914273A1, US20200316226A1, US20120315252A1, US20170360842A1, and references therein, and the like.

Briefly, methods for preparing exosomes can include the steps of: culturing cells in media, isolating the cells from the media, purifying the exosome by, e.g., sequential centrifugation, and optionally, clarifying the exosomes on a density gradient, e.g., sucrose density gradient. In some instances, the isolated and purified exosomes are essentially free of non-exosome components, such as cellular components or whole cells. Exosomes can be resuspended in a buffer such as a sterile PBS buffer containing 0.01-1% human serum albumin. The exosomes may be frozen and stored for future use.

Exosomes can be collected, concentrated and/or purified using methods known in the art. For example, differential centrifugation has become a leading technique wherein secreted exosomes are isolated from the supernatants of cultured cells. This approach allows for separation of exosomes from larger extracellular vesicles and from most non-particulate contaminants by exploiting their size. Exosomes can be prepared as described in a wide array of papers, including but not limited to, Fordjour et al., “A shared pathway of exosome biogenesis operates at plasma and endosome membranes”, bioRxiv, preprint posted Feb. 11, 2019, at https://www.biorxiv.org/content/10.1101/545228vl; Booth et ah, “Exosomes and HIV Gag bud from endosome-like domains of the T cell plasma membrane”, J Cell Biol., 172:923-935 (2006); and, Fang et ah, “Higher-order oligomerization targets plasma membrane proteins and HIV gag to exosomes”, PLOS Biol., 5: el58 (2007). Exosomes using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et ah, Journal of Extracellular Vesicles, 2:22614 (2013); Ono et ah, Sci Signal, 7 (332): ra63 (2014) and U.S. Application Publication Nos. 2012/0093885 and 2014/0004601. Methods for isolating exosome from cardiosphere-derived cells are found in, e.g., Ibrahim et al., “Exosomes as critical agents of cardiac regeneration triggered by cell therapy,” Stem Cell Reports, 2014. Specific methodologies include ultracentrifugation, density gradient, HPLC, adherence to substrate based on affinity, or filtration based on size exclusion.

Size exclusion allows for their separation from biochemically similar, but biophysically different microvesicles, which possess larger diameters of up to 1,000 nm. Differences in flotation velocity further allows for separation of differentially sized exosomes. In general, exosome sizes will possess a diameter ranging from 30-200 nm, including sizes of 40-100 nm. Further purification may rely on specific properties of the particular exosomes of interest. This includes, e.g., use of immunoadsorption with a protein of interest to select specific vesicles with exoplasmic or outward orientations.

Among current methods, e.g., differential centrifugation, discontinuous density gradients, immunoaffinity, ultrafiltration and high-performance liquid chromatography (HPLC), differential ultracentrifugation is the most commonly used for exosome isolation. This technique utilizes increasing centrifugal force from 2,000×g to 10,000×g to separate the medium- and larger-sized particles and cell debris from the exosome pellet at 100,000×g. Centrifugation alone allows for significant separation/collection of exosomes from a conditioned medium, although it may be insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/mL) or application of a discrete sugar cushion in preparation.

Ultrafiltration can be used to purify exosomes without compromising their biological activity. Membranes with different pore sizes-such as 100 kDa molecular weight cutoff (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. HPLC can also be used to purify exosomes to more uniformly sized particle preparations and preserve their biological activity as the preparation is maintained at a physiological pH and salt concentration. Other chemical methods have exploit differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes, although re-suspension of pellets prepared via this technique may be difficult. Flow field-flow fractionation (FIFFF) is an elution-based technique that is used to separate and characterize macromolecules (e.g., proteins) and nano-to micro-sized particles (e.g., organelles and cells) and which has been successfully applied to fractionate exosomes from culture media.

Beyond these techniques relying on general biochemical and biophysical features, focused techniques may be applied to isolate specific exosomes of interest. This includes relying on antibody immunoaffinity to recognizing certain exosome-associated antigens. As described, exosomes further express the extracellular domain of membrane-bound receptors at the surface of the membrane. This presents an opportunity for isolating and segregating exosomes in connection with their parental cellular origin, based on a shared antigenic profile. Conjugation to magnetic beads (e.g., such as anti-CD81 magnetic beads), chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

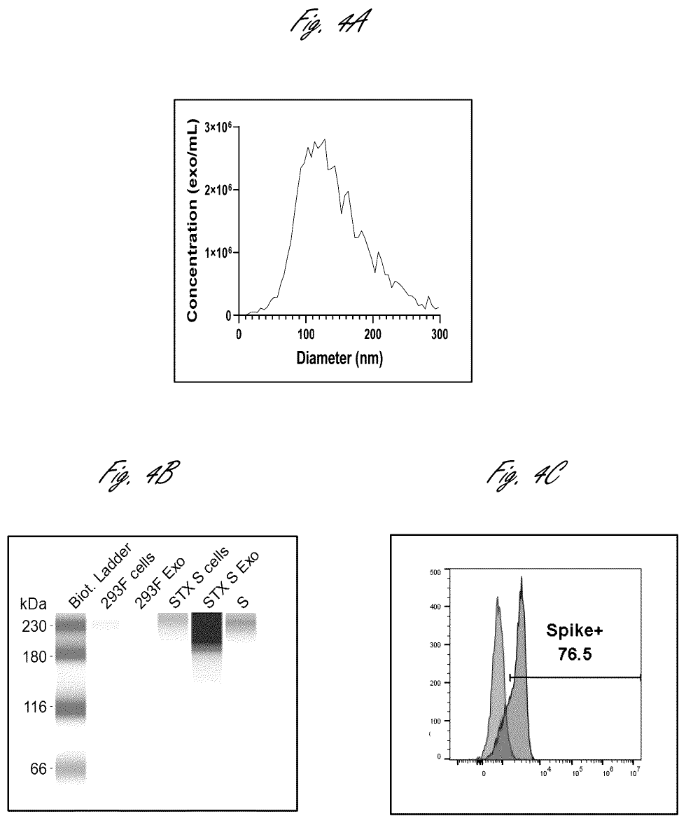

For example, exosomes (and other extracellular vesicles) may be produced via 293F cells. The 293F cells may be transfected with (or transduced with a lentivirus bearing) a polynucleotide that encodes a spike protein or a nucleocapsid protein, or chimeral fusions thereof, as described herein (see FIG. 3A), and expressing the spike protein or nucleocapsid protein, such that the spike protein or nucleocapsid is sorted into and displayed in or on the exosomes isolated therefrom. An example procedure for making exosomes from 293F cells may include steps as follows: 293F cells (Gibco™, Cat. #51-0029, ThermoFisher Scientific, Waltham, MA) may be tested for pathogens and found to be free of viral (cytomegalovirus, human immunodeficiency virus I and II, Epstein Barr virus, hepatitis B virus, and parvovirus B19) and bacterial (Mycoplasma) contaminants. Cells may be maintained in FreeStyle™ 293 Expression Medium (Gibco, Cat. #12338-018, ThermoFisher Scientific, Waltham, MA) and incubated at 37° C. in 8% CO2. For exosome production, 293F cells may be seeded at a density of 1.5E6 cells/ml in shaker flasks in a volume of about ¼ the flask volume and grown at a shaking speed of about 110 rpm. HEK293 cells may be grown in Dulbecco's modified Eagle's medium (DMEM) supplemented with 10% fetal calf serum.

To purify exosomes, the 293F cells may be cultured in shaker flasks for a period of three days. Cells and large cell debris may be removed by centrifugation at 300×g for 5 minutes followed by 3,000×g for 15 minutes. The resulting supernatant may be passed through a 0.22 μm sterile filtration filter unit (Thermo Fisher, Cat. #566-0020) to generate a clarified tissue culture supernatant (CTCS). The CTCS may be concentrated by centrifugal filtration (Centricon Plus-70, Ultracel-PL Membrane, 100 kDa size exclusion, Millipore Sigma, Cat. #UFC710008, St. Louis, MO), with about 120 mL CTCS concentrated to about 0.5 mL. Concentrated CTCS my then be purified by size exclusion chromatography (SEC) in 1×PBS (qEV original columns/35 nm: Izon Science, Cat. #SP5), with the exosomes present in each 0.5 ml starting sample eluting in three 0.5 ml fractions. Purified exosomes may be reconcentrated using Amicon® Ultra-4 100 kDa cutoff spin columns (Cat. #UFC810024). This process may yield a population of exosomes/small EVs that have the expected ultrastructure and size distribution profile of human exosomes and contain the exosomal marker proteins CD9 and CD63, at a concentrating effect of about 500-fold, to a final concentration of 1E10-2E12 exosomes/ml. The concentration and size of the isolated extracellular vesicles may be measured using NANOSIGHT nanoparticle tracking analysis system (Malvern Panalytical, Malvern, UK).

### SARS-CoV-2 Proteins

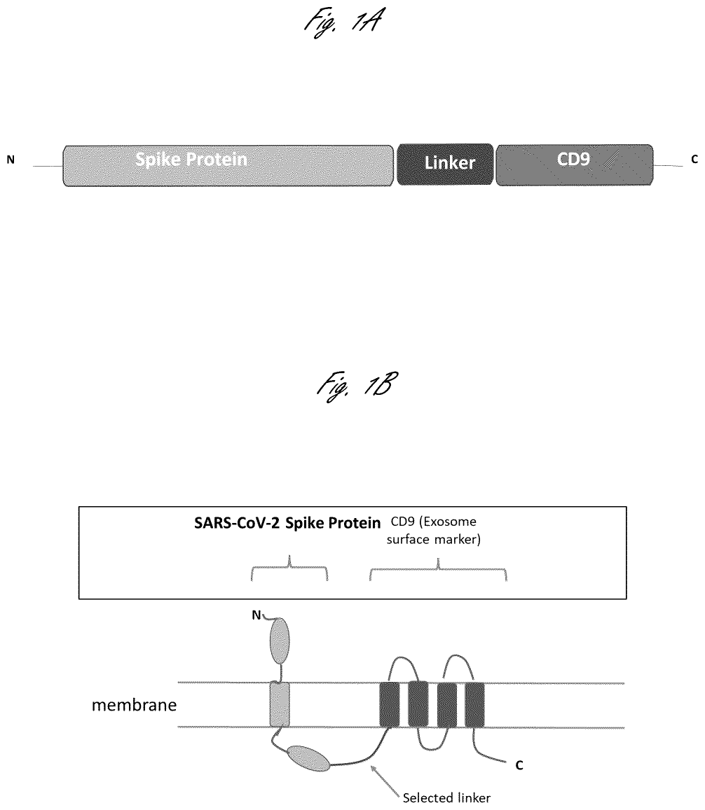

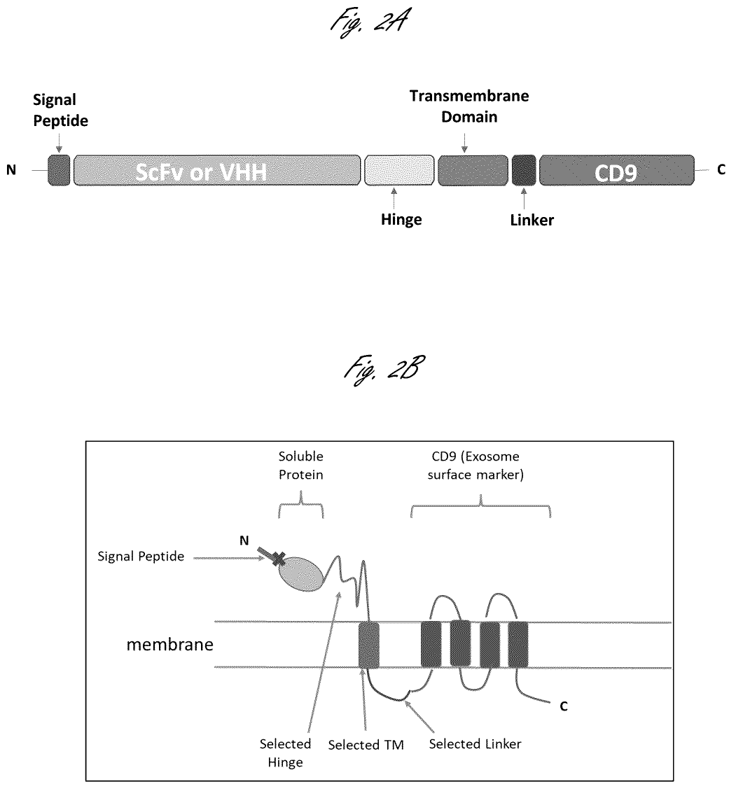

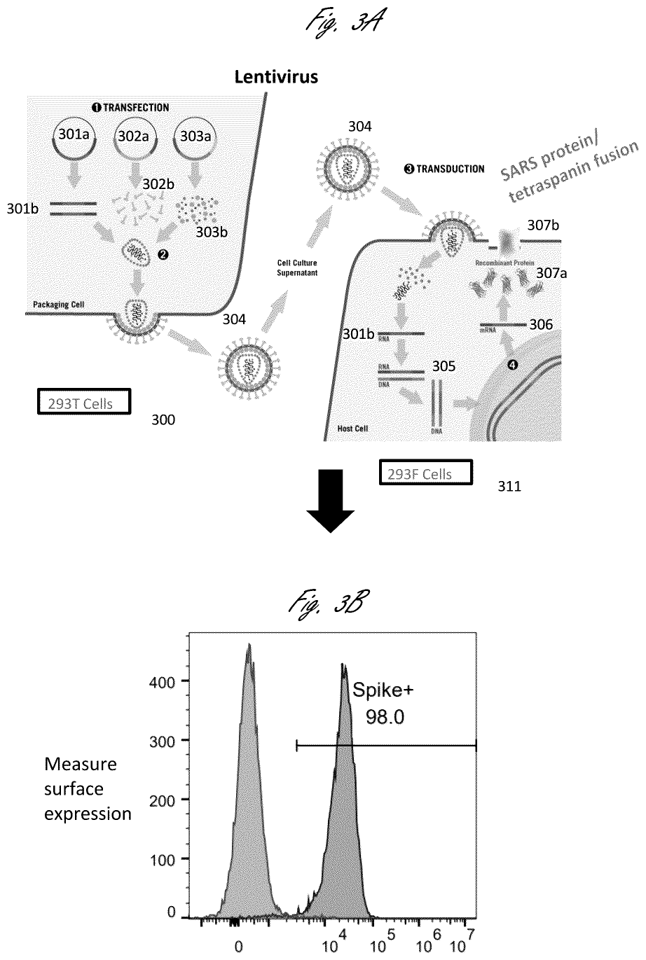

Disclosed are membrane-bound vesicles that contain one or more populations of SARS-CoV-2 structural proteins. By contain, what is meant is that the contained protein may be within the lumen of the vesicle, displayed on the surface of the vesicle, or both within the lumen and on the surface. Here, those fusion proteins containing a tetraspanin protein polypeptide sequence are mostly displayed on the surface of the vesicle. Those proteins that are displayed on the surface of the vesicle may have a portion of the protein inside the lumen, a portion of the protein spanning the membrane of the vesicle, i.e., a transmembrane spanning region or domain, and a portion of the protein extending outside the vesicle. In one embodiment, the SARS-CoV-2 structural protein is a spike glycoprotein(S), a nucleocapsid (N) protein, a membrane (M) protein, or an envelope (E) protein, or any combination thereof. See Satarker and Nampoothiri, “Structural Proteins in Severe Acute Respiratory Syndrome Coronavirus-2,” Arch Med Res. 2020 August; 51 (6): 482-491. See, e.g., FIGS. 1A, 1B, 2A, and 2C, which depict spike/CD9 and nucleocapsid/CD fusion proteins for expression of spike or nucleocapsid antigen on the surface of the exosomes.

In one embodiment, the antigenic protein is SARS-CoV-2 spike glycoprotein (a.k.a. spike protein or simply “spike”). The spike protein can be of any variant of SARS-CoV-2, such as, e.g., the Wuhan-1 strain, an omicron variant (e.g., BA.2 variant), a delta variant (e.g., B.1.617.2, AY.3, AY.103, AY.44, AY.43 variant, or the like), and epsilon variant (e.g., B.1.427 or B.1.429 variant), or any variant now known or yet to be discovered. As used herein, the term spike refers to any SARS-CoV-2 spike glycoprotein, chimera, or fragment thereof unless otherwise specified.

In some embodiments, the SARS-CoV-2 spike protein is the Wuhan-1 strain SARS-CoV-2 spike protein or a Delta variant SARS-CoV-2 spike protein; a furin-blocked, trimer-stabilized form of the Wuhan-1 strain SARS-CoV-2 spike protein; the Wuhan-1 strain SARS-CoV-2 spike protein with an amino acid change of D614G; the Wuhan-1 strain SARS-CoV-2 spike protein with di-proline substitutions of 986KV987-to-986PP987 (S-2P); and/or the Wuhan-1 strain SARS-CoV-2 spike protein with cleavage site mutations of 682RRAR685-to-682GSAG685, or equivalent (S-CSM).

### Extracellular Vesicles Displaying Spike Protein or a Soluble Protein (ScFv/VHH)

In one embodiment, the invention provides extracellular vesicles that express on their surface (a.k.a. “display”) spike protein or another targeting moiety such as an ScFv or VHH. The spike protein may be a delta variant with any one or more of trimer stabilization mutation, a prefusion conformation stabilization mutation (e.g., di-proline stabilization mutations), and a furin cleavage site mutation. See, e.g., Walls et al., “Structure, Function, and Antigenicity of the SARS-CoV-2 Spike Glycoprotein,” Cell 180, 281-292, Apr. 16, 2020; Wrapp et al., “Cryo-EM structure of the 2019-nCoV spike in the prefusion conformation,” Science 367, 1260-1263 (2020) 13 Mar. 2020; Kirchdoerfer et al., “Stabilized coronavirus spikes are resistant to conformational changes induced by receptor recognition or proteolysis,” Sci Rep 8, 15701 (2018), doi.org/10.1038/s41598-018-34171-7; Pallesen et al., “Immunogenicity and structures of a rationally designed prefusion MERS-CoV spike antigen,” PNAS, E7348-E7357, published online Aug. 14, 2017 pnas.org/cgi/doi/10.1073/pnas.1707304114; Juraszek, et al., “Stabilizing the closed SARS-CoV-2 spike trimer,” Nat Commun 12, 244 (2021), doi.org/10.1038/s41467-020-20321-x; Johnson, 2020; and Xiong 2020; and references therein.

Here, synthetic fusion proteins containing a C-terminal tetraspanin protein and either a SARS-CoV-2 spike protein (FIGS. 1A and 1B) or an ScFv or VHH protein (FIGS. 2A and 2B) were produced. It is important to note that ScFv and VHH proteins are soluble proteins, therefore the engineered fusion protein that includes tetraspanin and other transmembrane domains enables the placement of ScFv or VHH protein at the vesicle surface to provide a readily accessible moiety for cell or tissue targeting (see FIG. 2B).

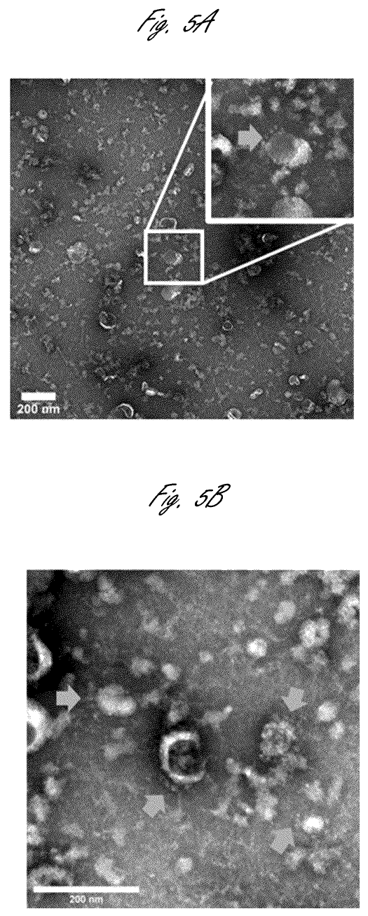

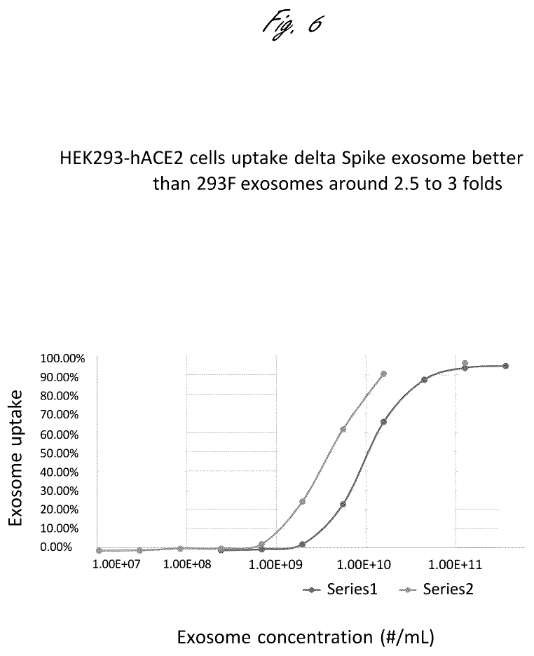

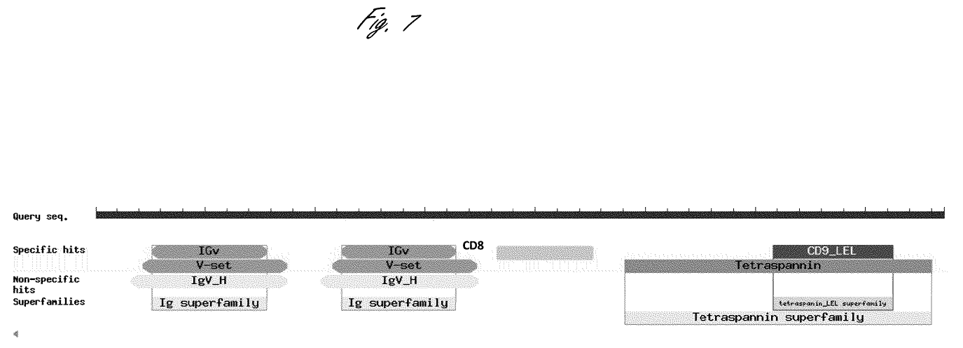

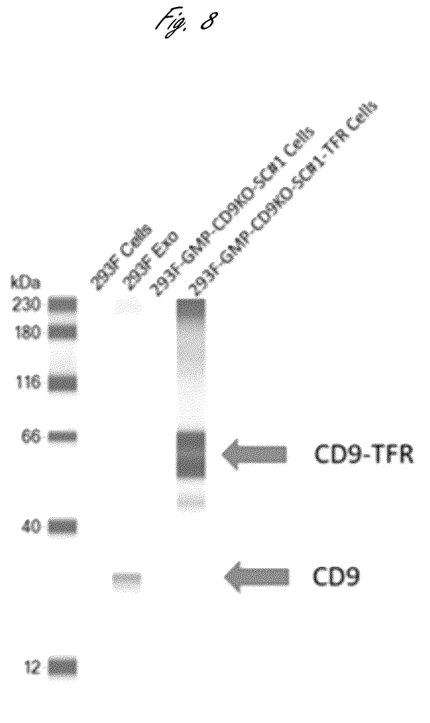

In one embodiment, the exosomes that express targeting protein on their surface were made from targeting moiety-expressing 293F cells. Turning to FIG. 3A, in specific exemplar embodiment, a packaging cell (300) was transfected with a plasmid that encodes the spike or ScFv/VHH-tetraspanin fusion protein (301a) and plasmids that encode lentivirus structural proteins (302a and 303a). The lentivirus proteins were produced (302b and 303b) and incorporated the fusion protein RNA to form a lentivirus vector containing the fusion protein RNA (304). A host cell (311) was transduced with the fusion protein RNA-bearing lentivirus (304), enabling the production of the SARS-CoV-2 spike or ScFv/VHH-tetraspanin fusion protein (307a), and its sorting to the plasma membrane (307b) (FIG. 3B) and the surface of exosomes that were produced by the transduced host cells (FIGS. 4C, 5A, and 5B).

Turning to FIGS. 4A-C and 5A and 5B, exosomes were isolated from 293F cells that harbored the spike-CD9 construct (FIG. 1A and FIG. 1B). FIG. 4A shows the size distribution of those exosomes from about 50 nm to about 270 nm, with a median of about 100-150 nm. FIG. 4B shows expression of spike-containing fusion protein in the transduced 293F cells (lane 4), and enriched expression/display of the spike-containing fusion protein in the exosomes that were derived from the transduced 293F cells (lane 5). FIG. 4C demonstrates significant expression of spike-containing fusion protein on those exosomes as determined by spike flow cytometry.

Transmission electron micrography confirmed that spike protein was expressed on the surface of the exosomes derived from the spike-CD9 transduced 293F cells (arrows at FIGS. 5A and 5B point to spikes at the surface of the vesicles).

### Targeting and Uptake of Spike-Expressing Exosomes

It is further envisioned that the exosomes could be engineered to selectively target organs or tissues of interest and allow safe and targeted delivery of drug cargos (e.g., ASOs, siRNAs, mRNAs, small drugs, antibodies and antibody fragments, and the like) to particular target cell, tissue, or organ.

In one example, HEK293 cells were engineered to express hACE2, a known pneumocyte receptor known to bind SARS-CoV-2 spike glycoprotein, and contacted with (i) exosomes produced by unengineered 293F cells or (ii) exosomes produced by 293F cells engineered to produce exosomes that present spike-CD9 fusion protein at the exosome surface.

Turning to FIG. 6, HEK293-hACE2 cells were seeded into 24-well plates and contacted with from about 1E7 to 1E12 exosomes per mL in reduced serum medium at about 37° C. for about 3.5 hr. In one series (series 2), the exosomes expressed spike-CD9. In another series (series 1), the exosomes did not express spike-CD9 or spike in any other form. Here, HEK293-hACE2 cells demonstrated better uptake of spike-expressing exosomes at about 2.5 to 3-fold lower concentration relative to “wildtype” 293F wt exosomes. See also Table 1.

Thus, spike-expressing exosomes may be used to deliver any drug cargo to lung or other tissues that express a spike receptor, such as ACE2.

TABLE 1
Uptake of Wildtype and Spike-Expressing
Exosomes by HEK293-hACE2 Cells

| 293T | | | | |
|---|---|---|---|---|
| Exosome @ | | Viability of | Delta | Viability of |
| #/m, 3x lot | | wt 293T | Spike | Delta Spike |
| (Lot. 18, | wt 293T | exosome- | Exosome | exosome- |
| 2.43E12, | Exosome | uptaken cells | uptake + | uptaken cells |
| 2.6 mg/ml) | uptake+ | (BD FVS780-) | (GG1-1) | (BD FVS780-) |
| 6E+11 | 96.07% | 84.68% | | |
| 2E+11 | 95.09% | 96.87% | 97.6% | 85.63% |
| 6.7E+10 | 88.9% | 87.1% | 86.61%* | 81.75%* |
| 2.2E+10 | 67.04% | 85.08% | 92.06% | 85.86% |
| 7.4E+9 | 24.29% | 86.24% | 63.24% | 86.02% |
| 2.5E+9 | 3.5% | 87.05% | 25.7% | 86.9% |
| 8.2E+8 | 0.92% | 85.5% | 3.66% | 87.22% |
| 2.7E+8 | 0.39% | 86.5% | 1.29% | 85.5% |
| 9.1E+7 | | | 1.26% | 86.28% |
| 3E+7 | | | 0.36% | 85.77% |
| 1E+7 | | | 0.21% | 84.93% |
| PBS | 0.66% | 85.03% | | |

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### Exosomes Expressing Transferrin Receptor Binding Moiety

The transferrin receptor (TfR) is a carrier protein for transferrin, and imports iron by internalizing the transferrin-iron complex through receptor-mediated endocytosis. In general, TfR1, the predominant TfR, is expressed at low levels on most normal cells. Increased expression is observed muscle cells. Therefore, in one embodiment, an anti-TfR nanobody can be used for targeting extracellular vesicles to muscle cells. In one embodiment, the extracellular vesicle engineered to express the TfR binding moiety is loaded with a cargo, such as a drug, RNA, DNA (e.g., an anti-sense oligomer [ASO]), a modified nucleic acid mimic such as a neutral charged antisense phosphorodiamidate morpholino oligomers (PMO ASO), or the like.

In one example, a chimeral anti-TfR1 nanobody (the TfR1 binding moiety) was designed and expressed in a HEK293 producer cells, and extracellular vesicles were produced having the moiety on their surface. In one embodiment, the TfR1 binding moiety-expressing vesicles were loaded with an ASO. The targeting of the vesicles to muscle cells was tested in vitro and in vivo, and the delivery of the ASO cargo to said target tissue and cells was demonstrated.

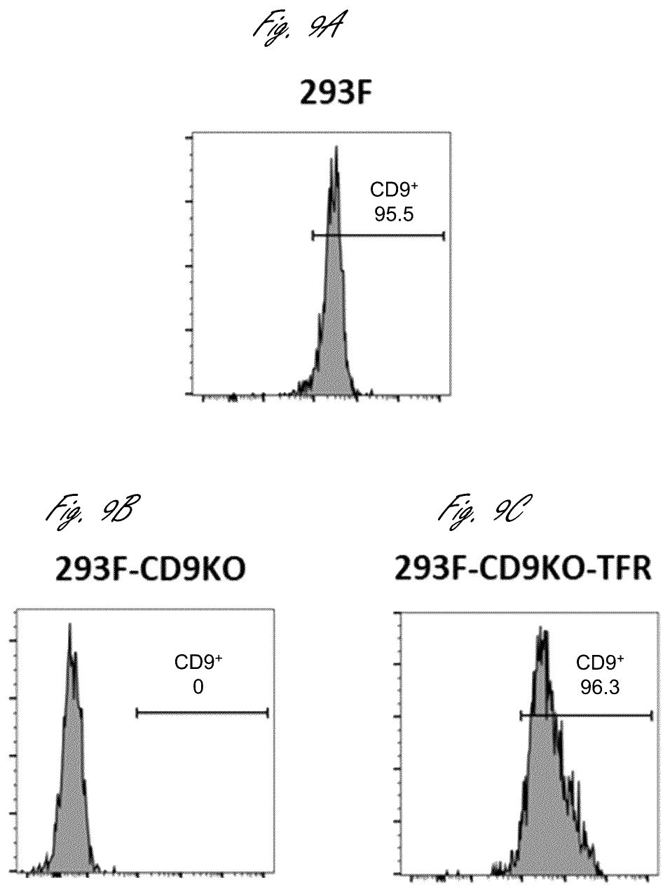

In one embodiment, the targeting moiety is an anti-TfR1 VHH construct proximate the N-terminus of a fusion protein that includes, from amino to carboxy, a first IgVH domain followed by a second IgVH domain followed by a CD8 domain with a transmembrane domain followed by a CD9 tetraspannin sequence, which is represented schematically in FIG. 7. In one embodiment, the targeting moiety includes an amino acid sequence that is (i) at least 80% identical to SEQ ID NO:1, (ii) identical to SEQ ID NO:1, (iii) at least 80% identical to SEQ ID NO:5, or (iv) identical to SEQ ID NO:5. In one embodiment, the targeting moiety-containing fusion protein includes an amino acid sequence that is (i) at least 80% identical to SEQ ID NO:2, (ii) identical to SEQ ID NO:2, (iii) at least 80% identical to SEQ ID NO:6, or (iv) identical to SEQ ID NO: 6.

In one embodiment, the exosome is produced from cells, such as e.g., 293 cells, containing a nucleic acid construct that encodes a targeting moiety having an amino acid sequence that is (i) at least 80% identical to SEQ ID NO:1, (ii) identical to SEQ ID NO:1, (iii) at least 80% identical to SEQ ID NO:5, or (iv) identical to SEQ ID NO:5. In one embodiment, the exosome is produced from cells, such as e.g., 293 cells, containing a nucleic acid construct that encodes a targeting moiety-containing fusion protein includes an amino acid sequence that is (i) at least 80% identical to SEQ ID NO:2, (ii) identical to SEQ ID NO:2, (iii) at least 80% identical to SEQ ID NO:6, or (iv) identical to SEQ ID NO:6. In one embodiment, the exosome is produced from cells, such as e.g., 293 cells, containing a nucleic acid construct having a nucleic acid sequence that is (i) at least 80% identical to SEQ ID NO:3, (ii) identical to SEQ ID NO:3, (iii) at least 80% identical to SEQ ID NO:7, or (iv) identical to SEQ ID NO:7. In one embodiment, the exosome is produced from cells, such as e.g., 293 cells, containing a nucleic acid construct having a nucleic acid sequence that is (i) at least 80% identical to SEQ ID NO:4, (ii) identical to SEQ ID NO:4, (iii) at least 80% identical to SEQ ID NO:8, or (iv) identical to SEQ ID NO:8.

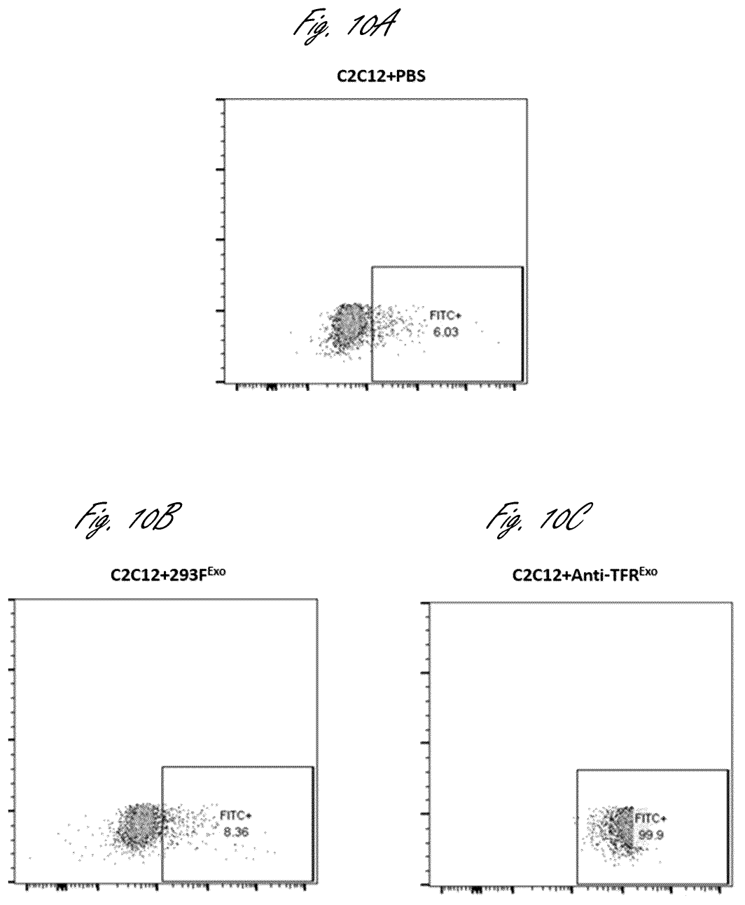

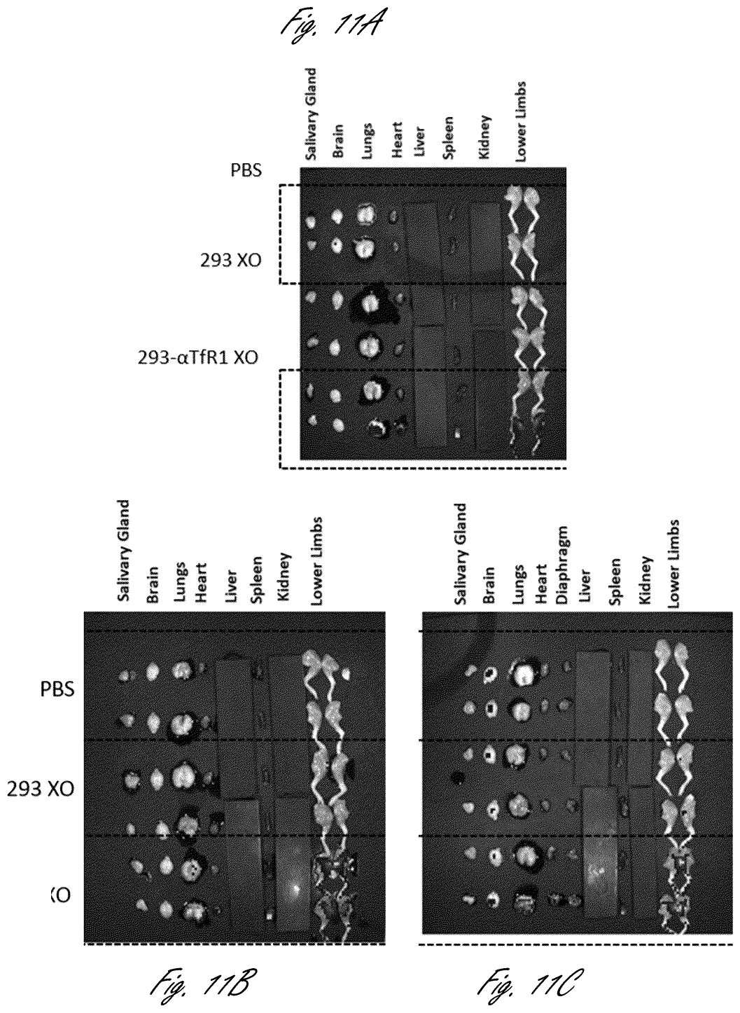

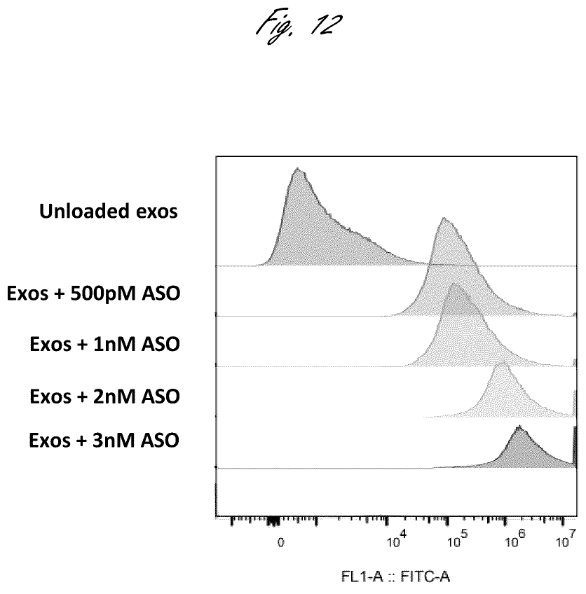

In one embodiment, one or more tetraspannin genes is/are knocked-out or knocked-down in the cell line from which the engineered exosomes expressing the targeting moiety CD9 fusion protein (see e.g., FIGS. 2A, 2B, and 7) are produced. In those embodiments in which the targeting moiety fusion protein contains a CD9 sequence, CD9-expression in the producer cell is knocked-out or knocked down. In some embodiments in which the producer cell produces the targeting moiety fusion protein of SEQ ID NO:2 or SEQ ID NO:6 or as depicted in FIGS. 2A, 2B, and 7, the host cell is a 293FΔCD9 cell (FIGS. 8 and 9A-D).

CD9+293F cells produce extracellular vesicles, more particularly exosomes, that express CD9, as shown in the western blot of FIG. 8, column 3 (CD9 band shown at wildtype 24 kD), and anti-CD9-labeled flow cytometry at FIG. 9, panel A (95.5% of CD9+293F cells and exosomes express CD9). In some embodiments, the cells used to produce the engineered exosomes expressing a targeting moiety fused to a CD9 sequence have the endogenous CD9 knocked-out (293F-CD9KO) to enable greater expression of the targeting moiety-CD9 fusion in the engineered exosomes. The western blot at FIG. 8 shows lack of detectable expression of endogenous CD9 in the 293F-CD9KO cells (column 4). When harboring and expressing an anti-TfR1-VHH-CD9 fusion protein, the 293F-CD9KO cell expresses the higher molecular weight fusion protein, which is detected with a CD9 antibody, and does not express the wildtype/endogenous CD9 protein at 24 kD (column 5). Likewise, the 293F-CD9KO cells and exosomes do not express CD9 as detected by flow cytometry (FIG. 9, panel B), but do express VHH-CD9 fusion protein in 293F-CD9KO-TFR cells and exosomes as detected by anti-CD9 antibodies by flow cytometry (FIG. 9, panel C).

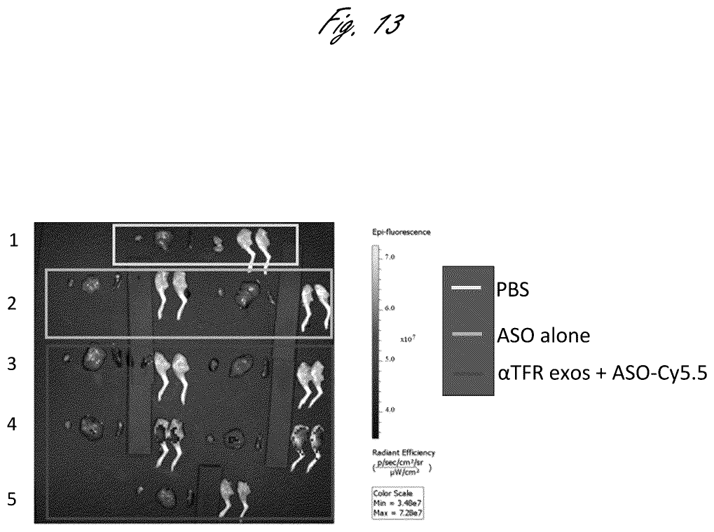

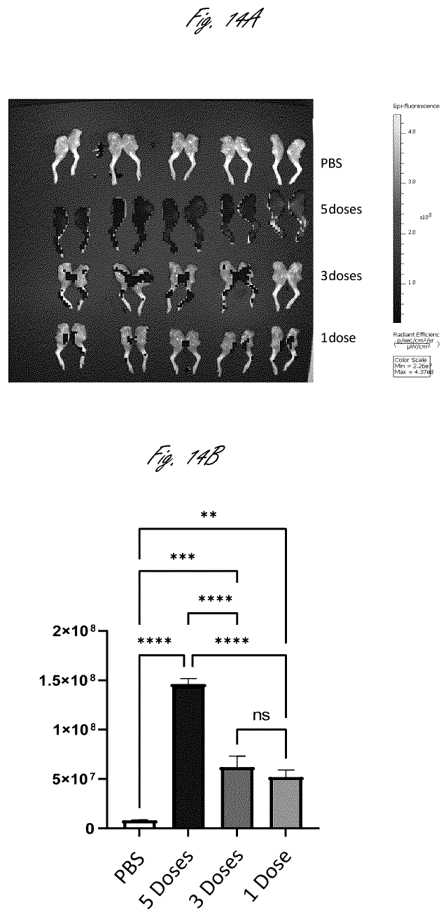

To test whether exosomes expressing anti-TfR1-VHH can target muscle cells, an immortalized mouse myoblast cell line, C2C12, was contacted with exosomes produced from a 293F-CD9KO-TFR engineered cell line and then subjected to anti-VHH flowcytometry. As shown in FIG. 10, panel C, C2C12 cells contacted with anti-TfR1-VHH expressing exosomes target and bind to contacted C2C12 murine muscle cells (compare to C2C12 cells contacted with PBS [panel A] and C2C12 cells contacted with non-engineered 293F exosomes [panel B]).

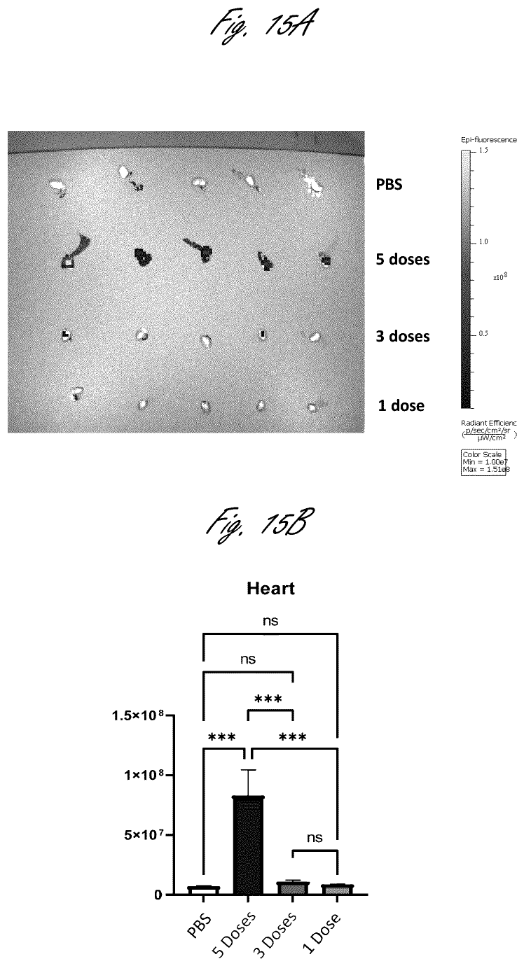

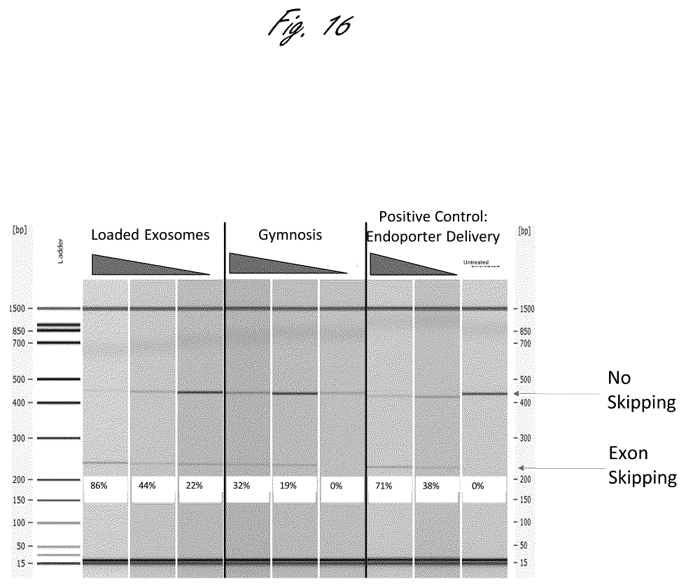

To test whether exosomes expressing anti-TfR1-VHH can target muscle cells in vivo, exosomes carrying an anti-TfR-VHH targeting moiety were labeled and intravenously injected into wild-type Balb/c female mice. Mouse tissues, including salivary glands, brains, lungs, heart, liver, spleen, kidney, and lower limbs, were imaged at 15 minutes, 30 minutes, 1 hour, 2 hours, 6 hours, and 24 hours post-injection. Three 24 hours images are shown in FIG. 11 (panels A-C), which demonstrate the targeting and decoration of lower limb muscle by the TfR1-targeted exosomes (bottom two rows of each image), but not non-engineered exosomes (middle two rows), or the PBS controls (top two rows). For both targeted and non-engineered exosomes, liver and kidney tissue, which are covered up in the images, showed signal, which is expected since the liver is a natural sink and kidney tissue a natural target for the HEK293 (kidney cells generate kidney-homing exosomes) exosomes. Thus, it was shown that exosomes produced from a 293F-CD9KO-TFR engineered cell line successfully target muscle tissue in vivo.

### Exosomes Expressing Transferrin Receptor Binding Moiety Delivers Fluorescently Labeled Anti-Sense Oligonucleotides (ASO) to Muscle Tissue

To test whether cargo-loaded exosomes expressing anti-TfR1-VHH can target and deliver cargo to muscle cells in vivo, exosomes loaded with a fluorescein amidite (FAM)-labelled antisense oligonucleotide (ASO) and carrying an anti-TfR-VHH targeting moiety were intravenously injected into wild-type Balb/c female mice.

Anti-TfR1 exosomes were loaded with increasing amounts of a Cy5.5-labeled ASO using a Calcium chloride2 heat shock method. Flow cytometry analysis demonstrated the successful loading of the anti-TfR1-expressing/TfR1 targeting exosomes with the ASO in a dose dependent manner (FIG. 12).

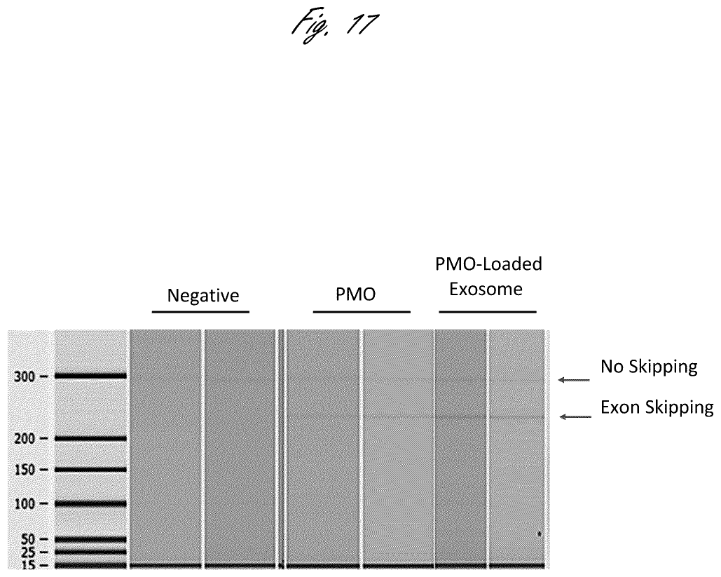

Anti-TfR1 exosomes loaded with Cy5.5-labeled ASO were injected into mice and after 24 hours tissues were imaged for Cy5.5 fluorescence to determine delivery of Cy5.5-labeled ASO into the tissues. As shown in FIG. 13, labeled ASO was successfully delivered by TfR1-targeted exosomes to hindlimb muscle (red boxed area, row 4), whereas ASO alone was not detected in hindlimb (green boxed area, row 2). Thus, it was shown that exosomes produced from a 293F-CD9KO-TFR engineered cell line and loaded with a labeled ASO successfully target muscle tissue and deliver the ASO cargo to muscle tissue in vivo. In some embodiments, Cy5.5-labeled ASO delivered in TfR-targeted exosomes demonstrated significant signal accumulation in murine hindlimb muscle at 5 doses (FIGS. 14A and 14B).

Anti-TfR1 exosomes loaded with Cy5.5-labeled ASO were injected into mice and after 24 hours heart tissues were imaged for Cy5.5 fluorescence to determine delivery of Cy5.5-labeled ASO into heart tissue. As shown in FIG. 15A, labeled ASO was successfully delivered by TfR1-targeted exosomes to heart muscle tissue (row 2) after 5 doses. At 5 doses, significant Cy5.5-ASO loading was detected in heart tissue (FIG. 15A, row 2; FIG. 15B showing quantitative histogram). Thus, it was shown that exosomes produced from a 293F-CD9KO-TFR engineered cell line and loaded with a labeled ASO successfully target heart muscle tissue and deliver the ASO cargo to heart muscle tissue in vivo.

### Exosomes Expressing Transferrin Receptor Binding Moiety Delivers Mouse Dystrophin Exon 23 Skipping PMO ASO to Murine Myoblasts and to Wild-Type Mice and Effectuates Exon 23 Skipping

Phosphorodiamidate morpholino oligomer (PMO) anti-sense oligomer (ASO) targeting exon 23 of murine dystrophin was loaded within the TfR1-targeting exosomes, and the loaded exosomes were delivered to murine myoblast cells in vitro. Controls included naked PMO ASO (gymnosis), and endoporter mediated transfection of the PMO ASO.

Here, dose dependent exon skipping was observed by all three approaches, with loaded exosomes showed the best overall exon skipping efficiency at all tested dosages (22% exon skipping with 2.4 uM PMO ASO-loaded TfR1-targeted exosomes, 44% exon skipping with 8 uM PMO ASO-loaded TfR1-targeted exosomes, and 86% exon skipping with 24 uM PMO ASO-loaded TfR1-targeted exosomes) (FIG. 16). Here also, 0% exon skipping was observed through gymnosis compared to 22% via exosomes at 2.4 uM PMO ASO. A comparable level of exon skipping was observed between the 8 uM loaded exosomes (44%) and 7.5 uM endoporter (38%) samples.

Further, it was observed that the murine dystrophin exon 23-skipping PMO ASO (i) alone and (ii) loaded in TfR1-targeted exosomes and delivered via either i.m. or i.v. lead to exon skipping in wild type mice skeletal muscle (FIG. 17).

### EMBODIMENTS

Various embodiments of the invention are disclosed, including the following embodiments.

Embodiment 1 provides an engineered vesicle comprising a fusion protein that comprises a first polypeptide sequence and a second polypeptide sequence, wherein: the fusion protein spans a membrane of a vesicle, the first polypeptide sequence comprises a sequence of a targeting moiety, the second polypeptide sequence comprises a sequence of a vesicle protein, and the targeting moiety is positioned outside of the vesicle.

Embodiment 2 provides an engineered vesicle of embodiment 1, wherein the targeting moiety comprises a receptor ligand, a receptor, an antibody, a heavy chain only antibody (VHH), a ScFv, a virus antigen, a virus antigen receptor, or fragments or chimeras thereof.

Embodiment 3 provides an engineered vesicle of embodiment 1 or embodiment 2, wherein the targeting moiety comprises a soluble protein, a type I transmembrane protein, or a type II transmembrane protein.

Embodiment 4 provides an engineered vesicle of any one of embodiments 1-3, wherein the targeting moiety binds specifically to no more than five tissues or cell-types.

Embodiment 5 provides an engineered vesicle of any one of embodiments 1-4, wherein the targeting moiety binds to muscle tissue, a muscle cell, or a muscle cell receptor.

Embodiment 6 provides an engineered vesicle of any one of embodiments 1-5, wherein the targeting moiety binds to an acetylcholine receptor, a transferrin receptor, a ryanodine receptor, a cholinergic receptor, a dystrophin, a myosin heavy chain, an alpha actinin, a PRAME family member 9, an FGF8, a protein phosphatase 1 regulatory subunit 27, an isopentenyl-diphosphate delta isomerase 2, a membrane integral NOTCH2 associated receptor 2, a SERCA2, an acetylcholine receptor epsilon, an SCN4A, a muscle specific creatine kinase (CK-MM), or a junctional sarcoplasmic reticulum protein 1.

Embodiment 7 provides an engineered vesicle of any one of embodiments 1-4, wherein the targeting moiety binds to lung tissue, a lung cell, or a lung cell receptor.

Embodiment 8 provides an engineered vesicle of any one of embodiments 1-4 and 7, wherein the targeting moiety binds to an ACE2 receptor, a surfactant protein, a secretoglobin family member, an advanced glycosylation end-product specific receptor, a membrane spanning 4-domains A15, a napsin A aspartic peptidase, a rhotekin 2, a solute carrier family 34 member, a mannose receptor C-type 1, a macrophage receptor, a mast cell expressed membrane protein, a mesothelin, or a periaxin.

Embodiment 9 provides an engineered vesicle of embodiment 6 or embodiment 8, wherein the targeting moiety is an antibody, an ScFv, or a VHH.

Embodiment 10 provides an engineered vesicle of any one of embodiments 1-4 and 7-8, wherein the targeting moiety is a virus glycoprotein.

Embodiment 11 provides an engineered vesicle of any one of embodiments 1-4, 7-8, and 10, wherein the targeting moiety comprises a coronavirus spike glycoprotein, an influenza hemagglutinin, an influenza neuraminidase, a respiratory syncytial virus F glycoprotein, or a respiratory syncytial virus G glycoprotein.

Embodiment 12 provides an engineered vesicle of any one of embodiments 1-4, 7-8, and 10-11, wherein the targeting moiety comprises a SARS-CoV-2 spike protein.

Embodiment 13 provides an engineered vesicle of any one of embodiments 1-12, wherein the vesicle protein is selected from the group consisting of Lamp-1, Lamp-2, CD13, Flotillin, Syntaxin −3, CD44, ICAM-1, Integrin alpha4, L1CAM, LFA-1, Vti-1A and B, CD9, CD37, CD53, CD63, CD81, CD82, CD151, ICAM-1 and tetraspanins.

Embodiment 14 provides an engineered vesicle of any one of embodiments 1-13, wherein the vesicle is an exosome.

Embodiment 15 provides an engineered vesicle of any one of embodiments 1-14, wherein the vesicle protein is a CD9 protein.

Embodiment 16 provides an engineered vesicle of any one of embodiments 1-15, wherein the fusion protein comprises, in order from amino terminus to carboxy terminus, a SARS-CoV-2 spike protein polypeptide, a linker polypeptide, and a CD9 polypeptide.

Embodiment 17 provides an engineered vesicle of any one of embodiments 1-15, wherein the fusion protein comprises, in order from amino terminus to carboxy terminus, a signal peptide, an ScFv or VHH protein polypeptide, a hinge region, a transmembrane domain polypeptide, a linker polypeptide, and a CD9 polypeptide.

Embodiment 18 provides an engineered vesicle of any one of embodiments 1-7 further comprising a cargo.

Embodiment 19 provides an engineered vesicle of embodiment 18, wherein the cargo is a fluorescent dye, a hydrophobic small molecule drug, a hydrophilic small molecule drug, a nucleic acid, a peptide, a peptide amino acid, an antibody or antibody fragment, or a contrast agent.

Embodiment 20 provides an engineered vesicle of embodiment 18 or 19, wherein the drug cargo is an antisense oligonucleotide (ASO) or a small interfering RNA (siRNA).

Embodiment 21 provides a vesicle comprising an exogenous polynucleotide.

Embodiment 22 provides a vesicle of embodiment 21, wherein the vesicle is an extracellular vesicle.

Embodiment 23 provides a vesicle of embodiment 22, wherein the extracellular vesicle is an exosome.

Embodiment 24 provides a vesicle of any one of embodiments 21-23, wherein the polynucleotide is an RNA.

Embodiment 25 provides a vesicle of embodiment 24, wherein the RNA is an mRNA.

Embodiment 26 provides a vesicle of embodiment 24, wherein the RNA is an siRNA.

Embodiment 27 provides a vesicle of embodiment 24, wherein the RNA is an miRNA.

Embodiment 28 provides a vesicle of any one of embodiments 21-23, wherein the polynucleotide is a DNA.

Embodiment 29 provides a vesicle of embodiment 28, wherein the DNA is a single-stranded oligonucleotide.

Embodiment 30 provides a vesicle of embodiment 29, wherein the single-stranded oligonucleotide is an anti-sense oligonucleotide (ASO).

Embodiment 31 provides a vesicle of embodiment 30, wherein the ASO is an exon-skipping anti-sense oligonucleotide.

Embodiment 32 provides a vesicle of embodiment 31, wherein the exon-skipping anti-sense oligonucleotide targets any one or more of exons 2-10 and 45-55 of the human dystrophin gene or murine analog thereof.

Embodiment 33 provides a vesicle of any one of embodiments 21-32, wherein the polynucleotide has an unmodified sugar-phosphate backbone.

Embodiment 34 provides a vesicle of any on of embodiments 21-32, wherein the polynucleotide has a chemical modification.

Embodiment 35 provides a vesicle of embodiment 34, wherein said chemical modification is any one or more of 2′-O-methyl phosphorothioate, phophorodithioate, methylphosphonate, and phosphorodiamidate modifications.

Embodiment 36 provides a vesicle of embodiment 34, wherein the polynucleotide is a phosphorodiamidate morpholino oligomer.

Embodiment 37 provides a vesicle of embodiment 34, wherein the polynucleotide is a phosphorodiamidate morpholino anti-sense oligomer.

Embodiment 38 provides a vesicle of any one of embodiments 21-37, wherein the polynucleotide is contained within the lumen of the vesicle.

Embodiment 39 provides a vesicle of any one of embodiments 21-37, wherein the polynucleotide is positioned on the outside of the vesicle.

Embodiment 40 provides a vesicle of embodiment 39, wherein the polynucleotide is conjugated to a moiety that binds an exosome surface marker protein.

Embodiment 41 provides a vesicle of any one of embodiments 21-40, wherein the vesicle is derived from a cardiosphere-derived cell (CDC) or a HEK293 cell or derivative thereof.

Embodiment 42 provides a method of making a vesicle of any one of embodiments 21-41 comprising contacting a vesicle with a polynucleotide, opening pores in a vesicle membrane, allowing the polynucleotide to enter the vesicle, and closing the pores, wherein the polynucleotide is contained within the vesicle.

Embodiment 43 provides a method of embodiment 42, wherein the pores are opened in the vesicle membrane by heat shocking the vesicle.

Embodiment 44 provides a method of embodiment 42, wherein the pores are opened in the vesicle membrane by freeze-thawing the vesicle.

Embodiment 45 provides a method of embodiment 42, wherein the pores are opened in the vesicle by sonicating the vesicle.

Embodiment 46 provides a method of embodiment 42, wherein the pores are opened in the vesicle by subjecting the vesicle to a voltage potential.

Embodiment 47 provides a method of embodiment 42 comprising initially combining the vesicle, Calcium chloride, and the polynucleotide; placing the initial combination on ice; exposing the combination to heat at 42° C.; and placing the heat exposed combination on ice to produce a recovered vesicle containing the polynucleotide.

Embodiment 48 provides a method of embodiment 47, wherein the Calcium chloride is at a concentration of 0.1M.

Embodiment 49 provides a method of embodiment 47 or 48, wherein the initial combination is placed on ice for about 40 minutes.

Embodiment 50 provides a method of any one of embodiments 47-49, wherein the heat exposure is for about 60 seconds.

Embodiment 51 provides a method of any one of embodiments 47-50, wherein the heat exposed combination is placed on ice for about 15 minutes.

Embodiment 52 provides a method of any one of embodiments 47-51 further comprising removing the Calcium chloride from the recovered combination.

Embodiment 53 provides a method of any one of embodiments 47-52, wherein the vesicle and the polynucleotide are first combined, and then the Calcium chloride is added to form the initial combination.

Embodiment 54 provides a method of embodiment 42 comprising initially combining the vesicle, Calcium chloride, and the polynucleotide; placing the initial combination on ice; freezing the combination; and thawing the frozen combination.

Embodiment 55 provides a method of embodiment 54, wherein the Calcium chloride is at a concentration of 0.1M.

Embodiment 56 provides a method of embodiment 54 or 55, wherein the initial combination is placed on ice for about 40 minutes.

Embodiment 57 provides a method of any one of embodiments 54-56, wherein the freezing is at −80° C. for about 15 minutes.

Embodiment 58 provides a method of any one of embodiments 54-57, wherein the thawing is a room temperature for about 10 minutes.

Embodiment 59 provides a method of any one of embodiments 54-58, wherein the freezing and thawing is repeated three times.

Embodiment 60 provides a method of any one of embodiments 54-59 further comprising removing the Calcium chloride from the thawed combination.

Embodiment 61 provides a method of any one of embodiments 54-60, wherein the vesicle and the polynucleotide are first combined, and then the Calcium chloride is added to form the initial combination.

Embodiment 62 provides a method of embodiment 45 comprising initially combining the vesicle and the polynucleotide; sonicating the combination; and placing the sonicated combination on ice.

Embodiment 63 provides a method of embodiment 47 comprising combining the vesicle and polynucleotide with Calcium chloride.

Embodiment 64 provides a method of embodiment 63 comprising removing the Calcium chloride from the sonicated combination.

Embodiment 65 provides a method of embodiment 46 comprising initially combining the vesicle and the polynucleotide; loading the combination into an electroporation cuvette; electroporating the combination; healing the electroporated combination on ice; centrifuging and filtering the healed electroporated combination.

Embodiment 66 provides a method of embodiment 65, wherein the polynucleotide comprises a cholesterol adduct.

Embodiment 67 provides a method of embodiment 65 or 66, wherein the combination comprises about 1-10 ug polynucleotide in a reaction volume of about 800 uL.

Embodiment 68 provides a method of any one of embodiments 65-67, wherein the combination comprises about 1E9-5E11 vesicles in a reaction volume of about 800 uL.

Embodiment 69 provides a method of any one of embodiments 65-68, wherein the electroporation cuvette has a 0.4 cm gap.

Embodiment 70 provides a method of any one of embodiments 65-69, wherein the electroporating comprises a voltage of 250-750 volts.

Embodiment 71 provides a method of any one of embodiments 65-70, wherein the electroporating comprises a capacitance of 50-125 uF.

Embodiment 72 provides a method of any one of embodiments 65-71, wherein the electroporating comprises a resistance of 200 ohms.

Embodiment 73 provides a method of any one of embodiments 65-72, wherein the healing is on ice for 10-30 minutes.

Embodiment 74 provides a method of any one of embodiments 65-73, wherein the centrifuging comprises about 3,000 RPM for about 1 minute in a tabletop centrifuge to remove arcing products.

Embodiment 75 provides a method of any one of embodiments 65-74, wherein the filtering comprises filtering the healed electroporated combination through a 0.22 uM filter.

Embodiment 76 provides a method of any one of embodiments 65-75, wherein the filtering further comprises filtering the healed electroporated combination through a 100 KDa filter.

Embodiment 77 provides a method of making a vesicle of any one of embodiments 21-41 comprising contacting a vesicle with a polynucleotide, wherein the polynucleotide adheres to the membrane of the vesicle.

Embodiment 78 provides a method of embodiment 77, wherein the polynucleotide is linked to a moiety that can bind to a vesicle protein.

Embodiment 79 provides a method of embodiment 78, wherein the moiety is CP05 and the vesicle protein is CD63.

Embodiment 80 provides a method of any one of embodiments 42-79, wherein the polynucleotide is siRNA, mRNA, anti-sense oligonucleotide (ASO), phosphorodiamidate morpholino oligomer (PMO), tagged ASO, and/or tagged RNA.

Embodiment 81 provides a method of any one of embodiments 42-80, wherein the polynucleotide is an ASO.

Embodiment 82 provides a method of embodiment 81, wherein the ASO is a PMO.

Embodiment 83 provides a method of embodiment 81 or 82, wherein the ASO targets a dystrophin exon.

Embodiment 84 provides a method of any one of embodiments 81-83, wherein the ASO is conjugated to a moiety that binds to an exosomal protein.

Embodiment 85 provides a method of any one of embodiments 81-84, wherein the ASO is an exon-skipping anti-sense oligonucleotide that targets any one or more of exons 2-10 and 45-55 of the human dystrophin gene.

Embodiment 86 provides a method of any one of embodiments 42-85, wherein the vesicle is an extracellular vesicle.

Embodiment 87 provides a method of embodiment 86, wherein the extracellular vesicle is an exosome.

Embodiment 88 provides a method of any one of embodiments 42-87, wherein the vesicle is obtained from cardiosphere-derived cells (CDCs) or HEK293 cells.

Embodiment 89 provides a method of treating a muscle disease comprising administering a vesicle of any one of embodiments 21-41 or a vesicle made by any one of embodiments 42-88 to a subject in need thereof.

Embodiment 90 provides a method of embodiment 89, wherein the muscle disease is a muscular dystrophy.

Embodiment 91 provides a vesicle of any one of embodiments 21-41 or made according to any one of embodiments 23-69 for use in treating a muscle disease.

Embodiment 92 provides a vesicle of embodiment 91, wherein the muscle disease is a muscular dystrophy.

### EXAMPLES

### Example 1: Electroporation

Here, electroporation can be performed at 150V, 50 uF, 200 Ohm; 400V; 250V, 125 uF; 400V, 125 uF; or the like, with a square wave or exponential volt delivery. Any electroporation apparatus may be used, including, for example, a BIORAD GENE PULSER XCELL TOTAL SYSTEM #1652660 (BIORAD, Hercules, CA), or the like. Here, also, the number of vesicles (i.e., exosomes) per reaction may be 1E9-5E11, including 5E10, 1E11, and 2.5E11 particles per reaction, containing about 2 ug polynucleotide (e.g., RNA, siRNA, mRNA, cholesterol tagged RNA, DNA, cholesterol tagged DNA, oligomers, ds oligomers, ss oligomers, ASO, PMO, and the like). Table 1 provides exemplar parameters.

TABLE 1
Electroporation Parameters

| Exosome | | | | |
|---|---|---|---|---|
| particles | Polynucleotide | Voltage | Capacitance | Delivery |
| 5E10 | 2 μg | 400 V | | Square wave |
| 2.5E11 | 2 μg | 400 V | | Square wave |
| 5E10 | 2 μg | 250 V | 125 uF | Exponential |
| 5E10 | 2 μg | 400 V | 125 uF | Exponential |
| 2.5E11 | 2 μg | 250 V | 125 uF | Exponential |
| 2.5E11 | 2 μg | 400 V | 125 uF | Exponential |

A specific exemplary electroporation workflow may include the following steps for example. The following workflow refers to NA (nucleic acid) and siRNA, but is applicable to any and all polynucleotides, including natural polymers and chemically modified polymers (e.g., PMOs and the like).
a. Loading reaction preparation
   i. Obtain 1×PBS and filter through 0.1 um filter. This is the loading buffer used to prepare all reaction with.
   ii. Prepare exosome work stock at 1.12E11 per 150 uL (7.47E8 ev/uL). This is constant per loading r×n.
   iii. Prepare nucleic acid (NA) (e.g., siRNA) work stock at 2 ug per 100 uL (20 ng/uL). The amount of NA to load can be scaled up per loading r×n.
   iv. Loaded Exosome Mix Prep: Prep mix containing 1.12E11 Exosomes+2 ug NA in 800 uL vol per electroporation r×n. (Do in triplicates per condition tested).
b. Loading reagent preparation
   i. Obtain ExoQuick-TC (SBI, Palo Alto, CA) to purify exosomes.
   ii. Exo-TC Prep for Electroporated Samples: 200 uL ExoTC+200 uL PBS per 800 uL electroporated sample per 1.7 mL Eppendorf tube.
   iii. Exo-TC Prep for siRNA only Spike-in Ctrl: 200 uL ExoTC+900 uL PBS per 100 uL siRNA (20 ng/uL) spike-in sample per 1.7 mL Epp. Tube.
      iv. Qiazol Prep for Spike-in Ctrl: 700 uL Qiazol per 1.7 mL Epp. Tube.
c. Spiking in controls
   i. Exo-TC Prep for Electroporated Samples: To 400 uL ExoTC+PBS, add 800 uL Electroporated samples in a dropwise fashion over reagent. 1.2 mL Final Vol. DO NOT MIX, INCUBATE IN 4 C O/N.
   ii. Exo-TC Prep for siRNA only Spike-in Ctrl:
   iii. TO 1100 uL ExoTC+PBS, add 100 μL of NA (e.g., siRNA) (20 ng/uL) in a dropwise fashion over reagent. 1.2 mL Final Vol. DO NOT MIX, INCUBATE IN 4 C O/N.
   iv. Qiazol Ctrl: To 700 uL Qiazol, add 150 μL of Exosomes (work Stock (7.47E8 ev/uL)+100 μL of siRNA (20 ng/uL). Mix and store in −80 C O/N.
d. Electroporation
   i. Obtain: Loaded Exosome Mix Prep and Electroporated NA (e.g., siRNA) only Mix Prep.
   ii. Obtain cuvettes (e.g., Gene Pulser/MicroPulser Electroporation Cuvettes, 0.4 cm gap #1652091, BIORAD, Hercules, CA).
   iii. Per cuvette, transfer 800 uL mix to be electroporated.
   iv. Electroporate using Gene Pulser Electroporator (BioRad), select parameter, e.g., 150V, 50 uF, 200Ω.
   v. Immediately post electroporation transfer cuvettes on ice to heal, 10-30 min.
   vi. Collect replicate electroporation r×ns in 15 mL conical tubes.
   vii. Centrifuge 3K RPM for 1 min to pellet arcing products (aggregates).
   viii. Transfer electroporated mix into new 15 ml conical tubes. Discard arcing products.
   ix. In dropwise fashion, transfer 800 uL aliquots of electroporated reactions onto Exo-TC reagent within 1.7 mL Eppendorf tubes (1.2 mL final vol). Incubate 4 C O/N.
   x. Transfer 800 μL aliquots of electroporated reactions into 1.7 mL Eppendorf tubes Proceed to in vitro cargo delivery or bank in −80 C until ready for in-vitro cargo delivery
e. Characterization post overnight incubation
   i. Bring down the temperature of a tabletop centrifuge to 4 C.
   ii. Obtain ExoTC treated samples and gently load into tabletop centrifuge. Centrifuge 2000 g for 20 min
   iii. Post centrifugation, gently remove 1000 uL solution. Centrifuge once more for 5 min in 4 C at 2000 g. Save extracted solution until end of experiment just in case it contains exosomes.
   iv. Post centrifugation, gently remove the remaining 200 uuL of solution. Save extracted solution until end of experiment just in case it contains exosomes.
   v. Transfer 700 uL Qiazol per tube containing exosomes or aggregated siRNA. Mix by vortexing for 10 sec. Spin down tubes. Bank in −80 C or proceed with RNA isolations.
   vi. Isolate RNA using miRNEasy Micro Kit as per Manufacturer's SOP.
   vii. Perform reverse transcription using TaqMan Small RNA Assays Kit.
   viii. Spike-in 10 ng of siRNA into RT plate.
   ix. Post RT, perform eight 10 fold serial dilutions from the 10 ng siRNA spike-in cDNA. Load in qPCR plate to generate a standard curve.
   x. Perform qPCR using TaqMan Probe provided in the TaqMan Small RNA Assays Kit. Use TaqMan Universal MasterMix II, No UNG PCR mastermix. Follow Manufacturer's SOP.
f. qPCR data analysis
   i. Obtain CT values (CT or cycle threshold is the number of cycles required for the fluorescent signal to cross the threshold, i.e., to exceed background level).
   ii. Obtain standard curve.
   iii. Use Linear Formula to obtain amount of percent siRNA recovered from all of the samples.
   iv. Normalize % siRNA recovered to that which was recovered by Qiazol. Qiazol is set at 100% recovery.
   v. Deduct the Exo TC spike in siRNA % recovered values from the remaining samples'% RNA recovery.
   vi. You now have the % loaded exosomes and % aggregated siRNA values.
   vii. Deduct the % aggregated values from the % loaded values, you now have the % Loading Efficiency.

### Example 2: siRNA Loading of Exosomes by Electroporation

Exosomes were isolated from cells (e.g., 293F cells, see, e.g., Tsai et al., JBC, 297 (5) 101266 (2021)) and loaded with siRNA according to the following procedure: (i) prepare exosomes to 1.4E8 p/uL in PBS (700 uL); (ii) prepare siRNA to 100 ng/uL in PBS (100 uL); (iii) combine exosomes and siRNA (800 uL); (iv) load into cuvette and then electroporate; (v) heal reaction mix on ice for about 30 minutes; (vi) centrifuge the reaction at 3,000 RPM for 1 minute; (vii) filter the reaction mix through 0.22 μm filter (Filter A); (viii) filter the reaction mix through 100 KDa filter (Filter B); and (ix) characterize the loaded exosome product prior to performing in vitro or in vivo cargo delivery.

In the examples presented here, the siRNA targets the ribonucleoside-diphosphate reductase subunit M2 (RRM2) gene and was specifically designed as follows: (i) cholesterol tag added to the 5′ end of the sense strand (5′-Chol-GGAGUGAUGUCAAGUCCAAUU-Cy3-3′) [SEQ ID NO. 9]; (ii) CY3 dye added to the 3′ end of the sense strand; (iii) UU overhangs on the 3′ ends of both the sense and antisense strands (5′-UUGGACUUGACAUCACUCCUU-3′) [SEQ ID NO. 10].

Various filtration processes may be employed. In one process (F01), exosomes were recovered after the first filtration step (Filter A), brought up to 350 μL and banked in −80° C. 800 uL PBS was added to the filter (A) and incubated overnight. The following day, the filter was vortexed for 15 sec, the banked samples were thawed and added to the 800 uL, and then the samples were centrifuged down to 250 uL. For further analysis, the samples were brought up to 350 uL for characterization and testing via in vitro cargo delivery (IVCD) (filtration sample 1 or F01).

In another process (F02), exosomes were recovered from the second filtration step (Filter B) immediately after centrifugation and then brought up to 350 uL and banked in −80° C. The following day, the exosomes were thawed, then characterized and tested via IVCD (filtration sample 2 or F02).

In another process (F03), exosomes were recovered after the second filtration step (Filter B), brought up to 350 μL and banked in −80° C. 800 uL PBS was added to the filter (A) and incubated overnight. The following day, the filter was vortexed for 15 sec, the banked samples were thawed and added to the 800 uL, and then the samples were centrifuged down to 250 uL. For further analysis, the samples were brought up to 350 uL for characterization and testing via in vitro cargo delivery (IVCD) (filtration sample 3 or F03).

In one example, 1.12E11 293F cell derived exosomes were loaded with 10 ug RRM2-CY3 siRNA via electroporation. Three different filtration procedures (F01, F02, F03) using 0.22 um Acrodisc filters (Filter A) and Amicon 100 KDa filters (Filter B) were used within a comparison study to rid the reactions of aggregated siRNA, siRNA arcing products, and free (not taken up by exosomes) siRNA. 50 uL per reaction underwent flow characterization. Here, it was demonstrated that RNA aggregates and arcing products were formed by electroporation. Those aggregates and arcing products were removed by all three filtration methods tested (F01, F02, and F03).

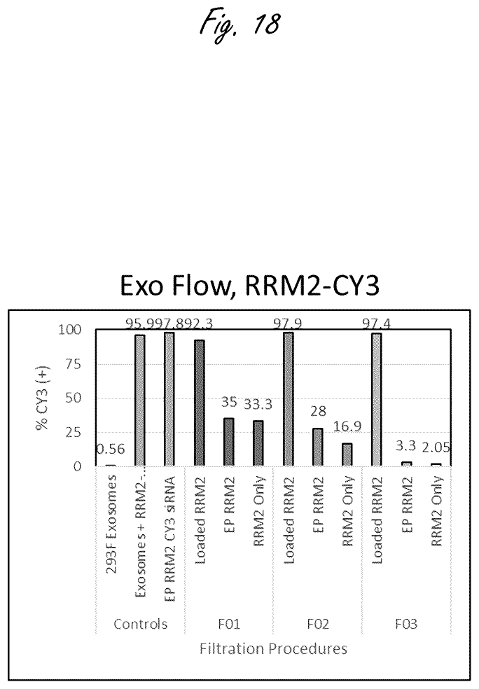

Here also, it was demonstrated that siRNA can be loaded into exosomes free of electroporation-induced siRNA aggregates after purification using any of the three filtration methods. It was observed that over 90% (or between 92% and 98%) of the exosomes were positive for RRM2-CY3 after purification (see FIG. 18).

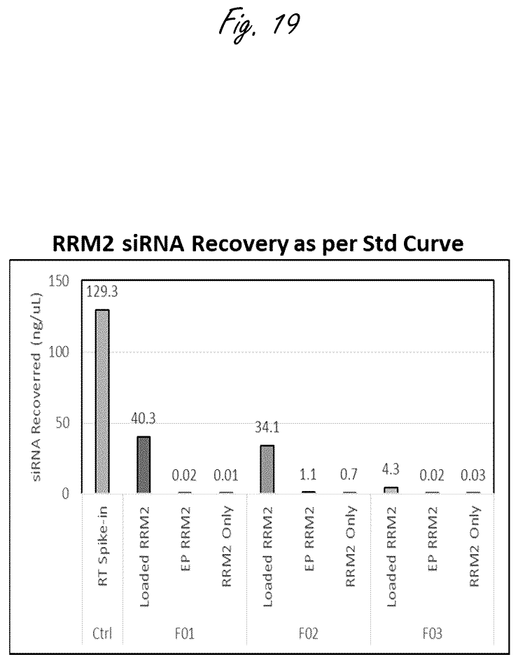

Exosomes loaded with the exemplar RRM2-Cy3 siRNA were demonstrated to knock down (KO) RRM2 gene expression in target cells. The filtration procedures removed both the aggregated and free RRM2 siRNA from the reaction mix to obtain only siRNA that is associated with the exosomes. Using a qPCR standard curve, the RRM2 concentration recovered from the loaded exosomes was observed to be about 40 ng/uL. FIG. 19 depicts the amount of siRNA recovered from the loaded exosomes based on the standard curve for each filtration process. Here, the exosomes from the F01 process were calculated to contain about 40 ng/uL, and the exosomes from the F02 process were calculated to contain about 34 ng/uL. Loading may be improved by increasing the number of exosomes per reaction, which may also improve target gene knock down in targeted cells.

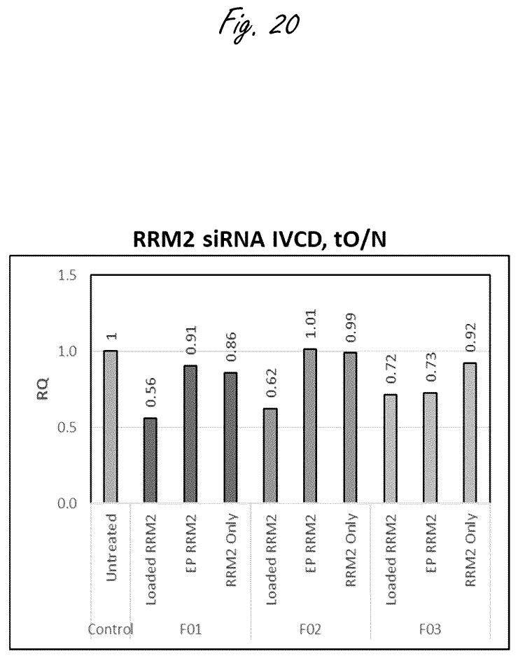

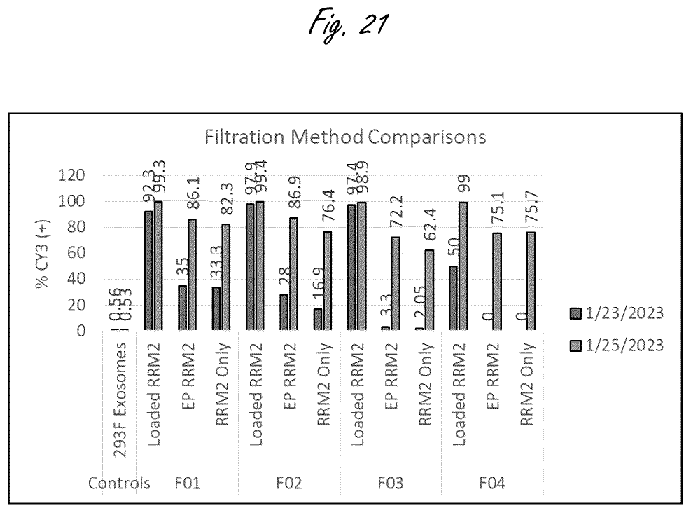

The siRNA-Cy3-loaded exosomes were contacted with cells in an in vitro cell delivery assay. FIG. 20. Here, RRM2 was calculated to be knocked down in target cells by about 50% to about 80% by siRNA-loaded exosomes.

In another example, 5.6E11 293F cell derived exosomes were loaded with 10 ug of the RRM2-CY3 siRNA via electroporation. As in the previous example, three different filtration procedures (F01, F02, F03) using 0.22 um Acrodisc filters (Filter A) and Amicon 100 KDa filters (Filter B) were used within a comparison study to rid the reactions of aggregated siRNA, siRNA arcing products, and free (not taken up by exosomes) siRNA. 50 uL per reaction underwent flow characterization. Here again, it was demonstrated that RNA aggregates and arcing products were formed by electroporation, and that those aggregates and arcing products were removed by all three of the filtration methods tested (F01, F02, and F03).

siRNA loading efficiency was tested. Here, 10 ug RRM2-CY3 siRNA was electroporated with or without the presence of 5.6E11 293F derived exosomes within an 800 uL reaction volume. All reactions were performed in 0.1 um filtered PBS. All aggregate and arcing products and free unbound siRNA were washed away using various methods of filtration. 10 ug RRM2-CY3 siRNA in an 800 uL reaction volume that did not undergo electroporation but did undergo filtration was also used as a control. 50 uL of the reaction was assayed via flow using the Beckman CytoFLEX cytometer. The percent loading was determined by flow and the results for this experiment are shown in FIG. 21. Here, all filtration methods (F01-F04) resulted in about 99% or greater exosomes loaded (loading efficiency) (˜92.3%-˜99.3% F01; ˜ 97.9%-˜99.4% F02; ˜97.4%-˜98.9% F03; ˜ 50%-˜899% F04).

Post IVCD gene knockdown caused by the siRNA-loaded exosomes was also assessed. Here, 10 ug RRM2-CY3 siRNA was electroporated with or without the presence of 5.6E11 293F-derived exosomes within an 800 uL reaction volume. All reactions were prepared in 0.1 um-filtered PBS. All aggregates and arcing products and free unbound siRNA were washed away using various methods of filtration (e.g., F01, F02, F03, F04). 10 ug RRM2-CY3 siRNA in an 800 uL reaction volume that did not undergo electroporation but did undergo filtration was also used as a control. 50 μL of the reaction mix was added to 50 uL of an ovarian carcinoma cell line (SKOV3) cells prepared at 200 cells/uL for a total of 10K cells/well. Cells were treated overnight and collected in buffer RLT/bME (QIAGEN) for RNA isolations and RT-PCR the following day.

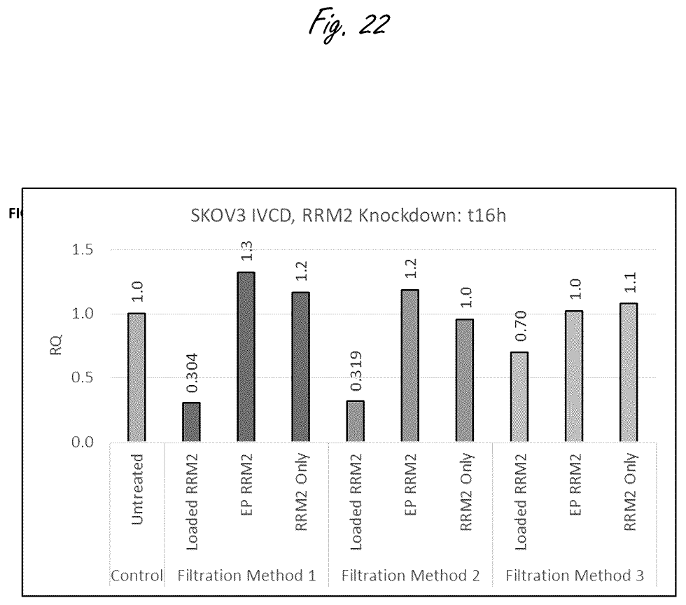

The results of the post IVCD RRM2 gene knockdown using the 5.6E11 293F-derived loaded exosomes are shown in FIG. 22. Here, two of the three filtration procedures (F01 and F02) yielded siRNA-loaded exosomes that suppressed the RRM2 gene by about 70%. It is important to note that all of the filtration procedures removed the free/unbound siRNA from the electroporated loaded exosome reactions.

### Example 3: Antisense Oligonucleotide (ASO) Loading of Exosomes by Electroporation

Exosomes were isolated from 293F cells and loaded with an antisense oligonucleotide DNA (ASO) according to the following procedure: (i) prepare exosomes to 1.4E8 particles/uL in PBS (700 uL); (ii) prepare ASO (polynucleotide) to 100 ng/uL in PBS (100 uL); (iii) combine exosomes and ASO (800 uL); (iv) load into cuvette and then electroporate; (v) heal reaction mix on ice for about 30 minutes; (vi) centrifuge the reaction at 3,000 RPM for 1 minute; (vii) filter the reaction mix through 0.22 μm filter (Filter A); (viii) filter the reaction mix through 100 KDa filter (Filter B); and (ix) characterize the loaded exosome product prior to performing in vitro or in vivo cargo delivery (IVCD).

In the examples presented here, the ASO targets the firefly luciferase gene (f-Luc) and was specifically designed as follows: 5′-GCG AAG AAG GAG AAT AGG GTT-3′/6-FAM/[SEQ ID NO. 11], wherein the 3′ end of the oligonucleotide is labeled with 6-carboxyfluorescein (6-FAM) to enable detection at ˜517 nm.

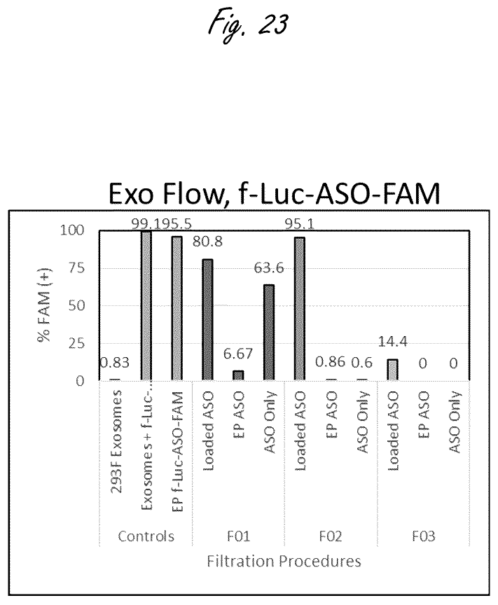

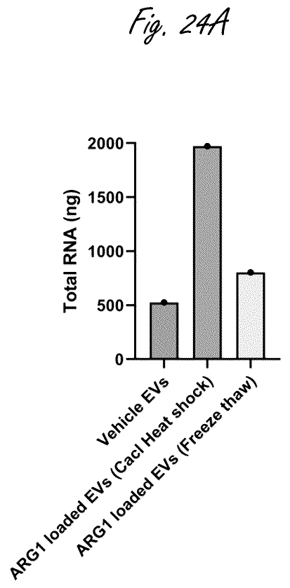

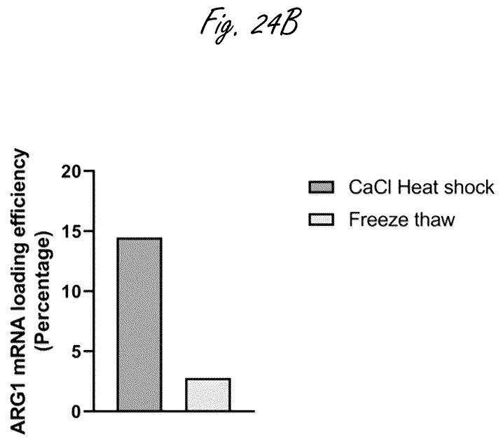

1.12E11 293F cell derived exosomes were loaded with 10 ug f-Luc-ASO-FAM via electroporation. Three different filtration procedures using 0.22 um Acrodisc filters and Amicon 100 KDa filters were used (i.e., F01, F02, and F03 as described herein) within a comparison study to rid the reactions of aggregated oligonucleotides and free unbound or unloaded oligonucleotides. Loading efficiency was determined by flow characterization of 50 uL reaction samples. FIG. 23 shows the percent FAM+ exosomes for each filtration method as determined by flow detection of fluorescein. Here, post filtration, over 90% of the exosomes were positive for the ASO (F02).

Here, it was discovered that exosomes can be loaded with siRNAs and/or ASOs at >90% loading efficiency using electroporation. Aggregated and free unbound siRNAs and ASOs can be washed away using filtration procedures where only loaded exosomes are recovered.

### Example 4: Nucleic Acid Loading of Exosomes by Calcium Chloride2 Heat Shock

Exosomes have been considered as drug carrier particles to use in vaccination and therapeutic approaches. Here we assessed Calcium chloride2 heatshock method for loading various lengths of nucleic acids from siRNA and ASO to mRNA in exosomes. ASO and siRNA emerged as a powerful tool for targeting mutant genes in various disorders including cancers and Duchenne Muscular Dystrophy. Developing mRNA delivery have enhanced in therapy and vaccine applications. Several mechanical and chemical approaches have been applied to load nucleic acids in exosomes. Using Calcium chloride2 could increase the intact loading of nucleic acids in exosomes and consequently their therapeutic efficiency.

Exosomes isolated from HEK293 cells were mixed with ASO and Calcium chloride2 on ice and followed with heat shock at 42° C. The unloaded nucleic acids and Calcium chloride2 were cleaned up by enzymatic digestion and filtration column. HEK293 cells were treated with siRNA loaded exosomes for 48 h. The expression of the target gene in cells treated with loaded siRNAs was measured using qPCR. Exosomes loaded with Cy5 labeled ASO were labeled with anti-DC9-APC antibodies to detect in flowcytometry compared to the unlabeled naked exosomes. Total RNA of exosomes loaded with mRNA were extracted to assay fragment sizes in bioanalyzer.

Exosomes loaded with dye labeled ASO showed a clear shift in FITC and PE axis of loaded exosomes samples compared to the negative controls. Shifting in different concentrations of loaded ASO run into flowcytometry confirmed the exosome loading. Analysis of HEK293 cells uptake siRNA loaded exosomes showed increase the silence of target gene up to more than 80% in 48 h. The electropherogram showed the presence of the full-length size of mRNA in loaded exosomes compared to the naked exosomes samples.

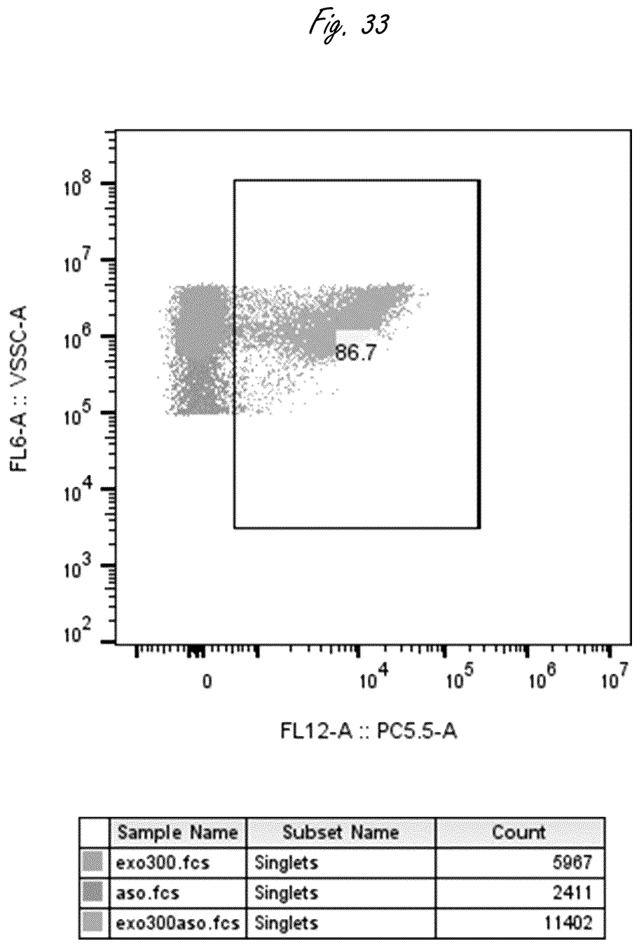

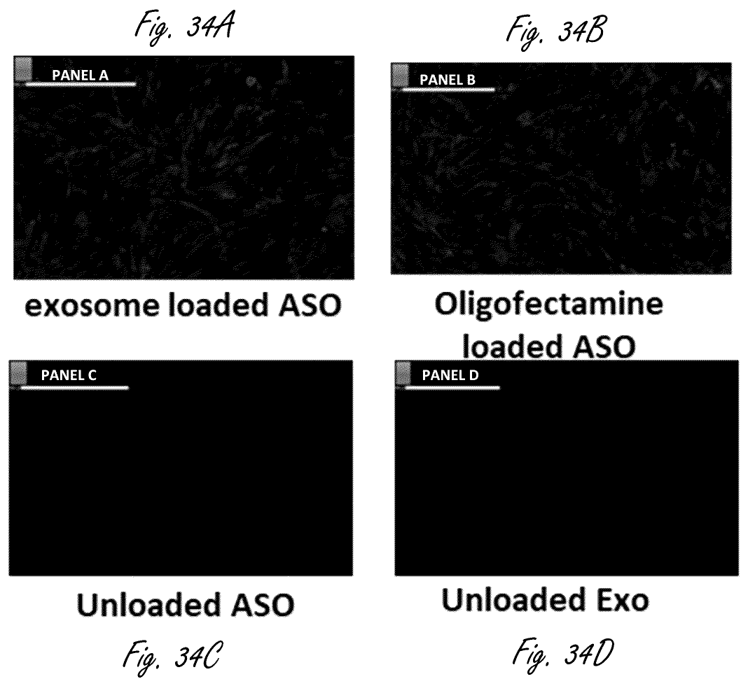

Turning to FIG. 33, HEK293F derived exosomes were loaded with Cy5-labeled ASO using the Calcium chloride2 heat shock method. The flowcytometry assay showed Cy-5 fluorescence to be associated with the exosome population (inset region indicating Cy-5-ASO-loaded exosome population at 86.7% loading efficiency), demonstrating loading of the ASO into the exosomes.

Turning to FIG. 34, panels A-D, the Cy5-labeled ASO-loaded exosomes were added to HEK293 cells in culture media. The uptake of the Cy5-labeled ASO via loaded exosomes was observed as shown by the intracellular localization of the cy5 signal (Panel A). This is comparable to the oligofectamine-ASO administered to cells (positive control, Panel B). Unloaded exosomes (Panel D) and ASO alone (Panel C) were used as negative control and show no uptake of ASO into the cells.

Similarly, FIG. 31 depicts the loading of HEK-293 cell-derived exosomes engineered to express a transferrin −1 receptor binding moiety (anti-TfR1 scFv or VHH) with from 500 pM to 3 nM FAM-labelled dystrophin exon-skipping ASO using the Calcium chloride2 heat shock method. Here again, successful loading of the ASO (here, a FAM-labeled dystrophin exon-skipping ASO) into exosomes by Calcium chloride2 heat shock is demonstrated by concentration-dependent increase in fluorescence via exosome flow cytometry (fluorescent intensity as a function of ASO concentration).

Similarly, FIG. 32 (and FIG. 49) demonstrates the muscle-specific targeting and delivery of the Cy5-labeled dystrophin exon-skipping ASO described above via muscle-targeting engineered exosomes. Here, the ASO-loaded exosomes carrying an anti-TfR targeting moiety were loaded with Cy5.5 labeled exon-skipping ASO and injected into wild-type Balb/c female mice by I.V., the tissue was imaged at 24 hours post I.V. injection. The tissue shown in FIG. 32, panel B are lower limb skeletal muscle. Here, 2 of 5 mice showed a good accumulation of labeled signal in the lower limbs. No muscle-targeting was observed in ASO-only (without exosomes) injected mice.

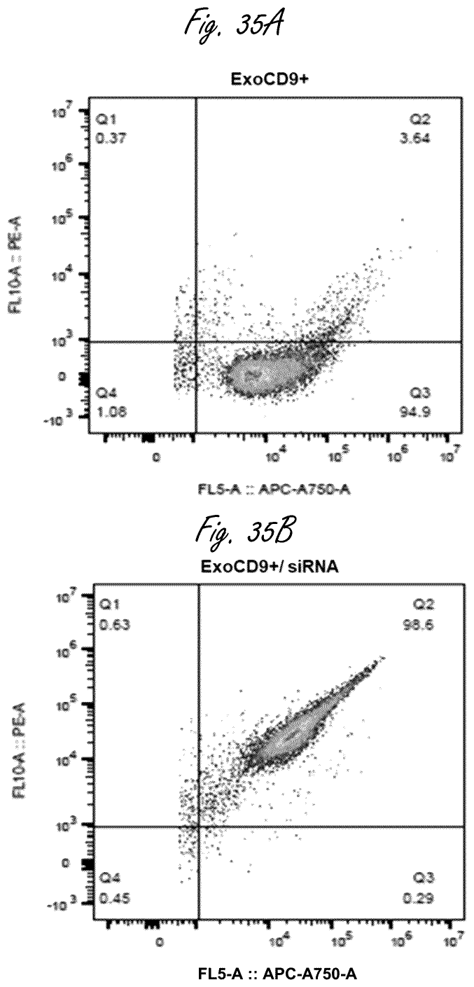

Turning to FIG. 35, HEK293F derived exosomes were loaded with luciferase message small interfering RNA (Luc-siRNA) using a Calcium chloride2 heat shock loading method, followed by RNase treatment to remove unloaded molecules. Panels A and B depict a flowcytometry assay with allophycocyanin (APC)-labeled anti-CD9 fluorescence on the Y-axis and phycoerythrin (PE)-labeled fLuc-siRNA fluorescence on the X-axis. Panel A depicts unloaded exosomes being positive for CD9 and negative for fLuc-siRNA (quadrant 3, 94.9%). Panel B depicts fLuc-siRNA-loaded exosomes showing a clear population of loaded exosomes positive for both APC CD9 (exosome marker) and PE siRNA (quadrant 4, 98.6%), confirming loading of the exosome with siRNA.

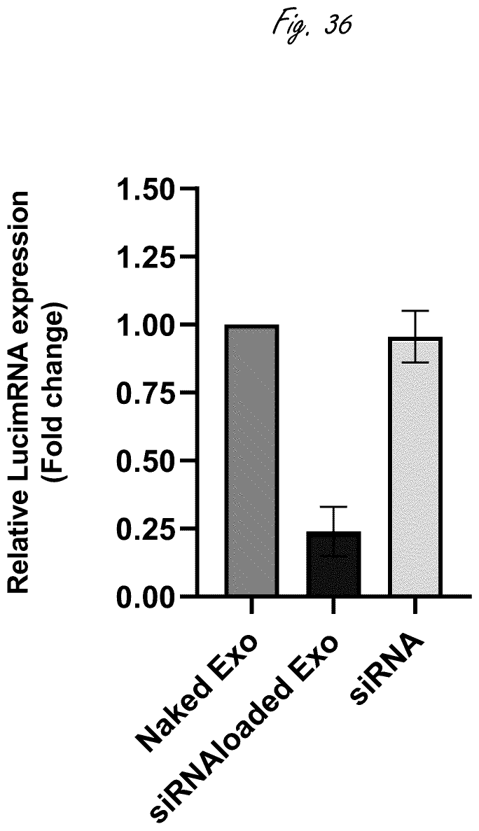

Turning to FIG. 36, the fLuc-siRNA loaded exosomes were added to media containing HEK293F-Luc cells. After 48 h, the fluc-mRNA expression was measured using qPCR. Here, significant reduction of fluc expression was observed in those HEK293F-Luc cells treated with the fLuc-siRNA-loaded exosomes (middle bar), thereby confirming the functional delivery of the siRNA by the fLuc-siRNA-loaded exosomes.

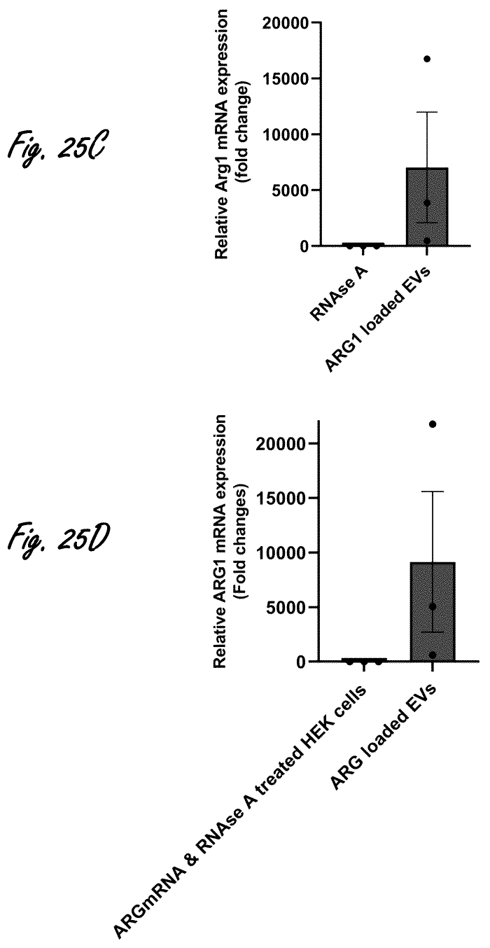

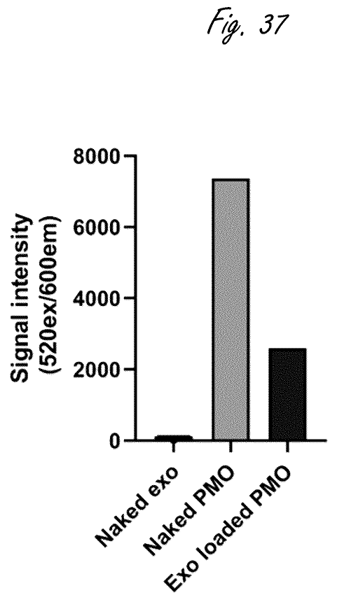

Turning to FIG. 37, HEK293F derived exosomes that were loaded with mRNA via a Calcium chloride2 heat shock method were treated with RNase to remove unloaded mRNA. Here, total RNA was isolated from mRNA loaded and control exosomes and analyzed by Bioanalyzer RNA analysis. An intact full-length mRNA was detected at about 2000 on the ladder, confirming loading of the mRNA (e.g., ARG1 mRNA) into the exosomes. At FIG. 25B, for example, mRNA-loaded exosomes were added to the HEK293F cell-containing media, the cells were then harvested after 24 h, and mRNA expression was assessed by qPCR. As shown, elevated expression of the mRNA (e.g., ARG1 mRNA) was detected, confirming functional delivery of mRNA by exosomes.

Here, loading exosomes with various nucleic acids, including but not limited to ASO, siRNA, and mRNA, was accomplished using Calcium chloride2 heat shock.

### Example 5: Exosomes Loading with PMO ASO

It is envisioned that phosphorodiamidate morpholino oligomers (PMO), including antisense phosphorodiamidate morpholino oligonucleotides, can be loaded into exosomes, such as 293F-derived exosomes, cardiosphere-derived cell (CDC)-derived exomes, skeletal muscle cell-derived exosomes, C2C12-derived exosomes, or any other exosomes derived from any other cell source, by electroporation, or by Calcium chloride2 heat shock or freeze thaw as described herein for native ASOs or other nucleic acid oligomers. It is envisioned that PMOs, such as PMO ASOs, are designed to effect exon skipping in target recipient cells to enable production of an internally deleted partially functional protein. Exon skipping PMO ASOs for use in treating Duchenne muscular dystrophy (DMD) or spinal muscular atrophy (SMA) patients include, for example, eteplirsen, which induces skipping of exon 51 of dystrophin, golodirsen and viltolarsen (a 25-mer oligomer that is 100% complementary to the target sequence 5′-GAACACCUUCAGAACCGGAGGCAAC-3′ [SEQ ID NO:12]), each of which induces skipping of exon 53 of dystrophin, and casimersen, which induces skipping of exon 45 of dystrophin to treat Duchenne muscular dystrophy (DMD) patients with eligible mutations, and nusinersen to treat spinal muscular atrophy (SMA). PMOs and there delivery into cells is described in Chapter 12: In Vitro Delivery of PMOs in Myoblasts by Electroporation, by Remko Goossens and Annemieke Aartsma-Rus, in Antisense RNA Design, Delivery, and Analysis, Arechavala-Gomeza V, Garanto A, editors, New York: Humana; 2022, which is incorporated herein by reference.

It is further envisioned that PMO ASOs (or other neutral charge oligomers-anti-sense or other) or natural ASOs can be loaded into any and all exosomes, such as, e.g., CDC-derived exosomes and 293F exosomes, by way of sonication, heat shock, freeze-thaw, Calcium chloride-included sonication, Calcium chloride-included heat shock, or Calcium chloride-included freeze-thaw, as described herein for other nucleic acid oligomers. It is further envisioned that neutral oligomers, such as PMO ASO, can be loaded into exosomes via passive diffusion with or without pore formation in the exosome membrane and with or without the addition of charged ions. For example, it is envisioned that neutral oligomers (e.g., PMO ASO) can be mixed with exosomes and allowing the oligomers to passively diffuse into the lumen of the exosome and/or associate with the exosome membrane to produce neutral oligomer loaded exosomes.

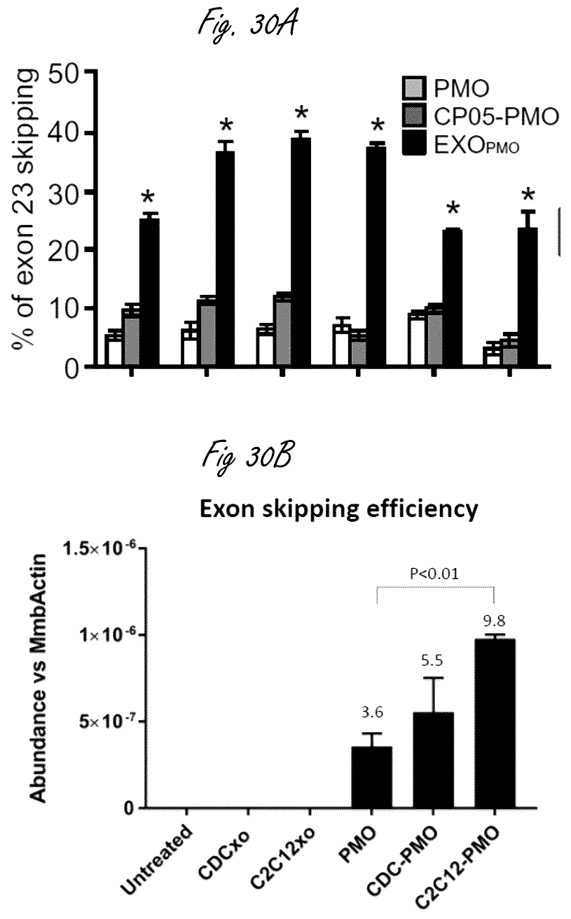

In one example, CDC-exosomes and C2C12-exosomes bearing a dystrophin exon 23-skipping PMO ASO, which targets the boundary of exon 22 and 23 in the murine DMD gene, effectively induced about 40% exon 23 skipping in target C2C12 cells after contact of the C2C12 cells with the exon 23 PMO ASO-bearing exosomes (FIGS. 30A and 30B).

In some embodiments, the PMO is attached to the exosome membrane by linking the PMO to a moiety (e.g., CP05) that binds to an exosomal surface marker (e.g., CD63), and combining the conjugated PMO (e.g., CP05-PMO) with the exosomes. Peptide CP05 is described in Gao, et al., “Anchor peptide captures, targets, and loads exosomes of diverse origins for diagnostics and therapy,” Science Translational Medicine, vol. 10, no. 444, 6 Jun. 2018, and Perez, et al., “Enhancing the Therapeutic Potential of Extracellular Vesicles Using Peptide Technology,” in: Langel, Ü. (eds) Cell Penetrating Peptides. Methods in Molecular Biology, vol 2383, pp 119-141. Humana, New York, NY.

In one embodiment, exosomes were loaded with a PMO ASO directed to exon 51 of the dystrophin gene (PMO ASO 51), e.g., a 3-prime lissamine labeled PMO ASO having a sequence of 5′-CT CCA ACA TCA AGG AAG ATG GCA TTT CT-3′ (SEQ ID NO. 13) (PMO ASO 51). Here, 293 exosomes were loaded with the PMO ASO using the Calcium chloride2 heat shock method disclosed herein. Alternatively, the PMO ASO may be loaded using heat shock but without including Calcium chloride2 in the process.

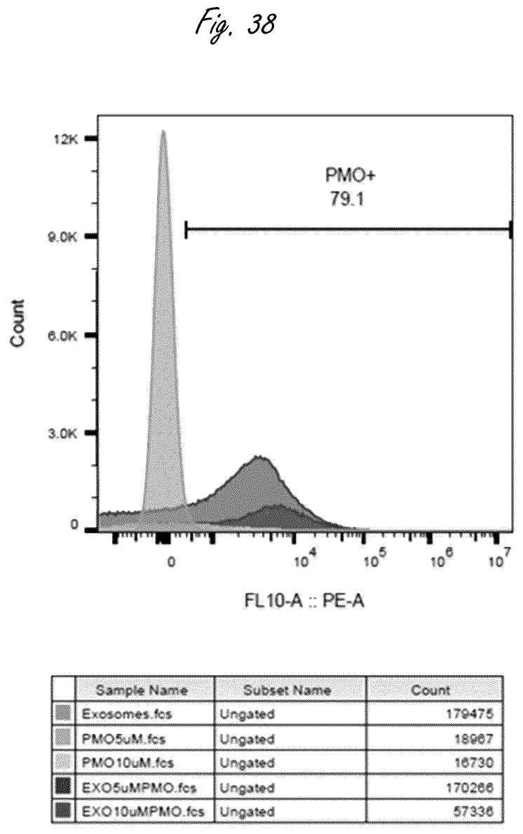

Here, FIG. 37 shows the results of a citation assay demonstrating 35% loading of the PMO ASO 51 in exosomes from Initial 0.6 uM PMO used for loading. (575-593 ex/em of Lissamine). FIG. 38 demonstrates that 79.1% of exosomes subjected to loading with either 5 uM or 10 UM of the subject PMO ASO 51 harbor the PMO ASO 51.

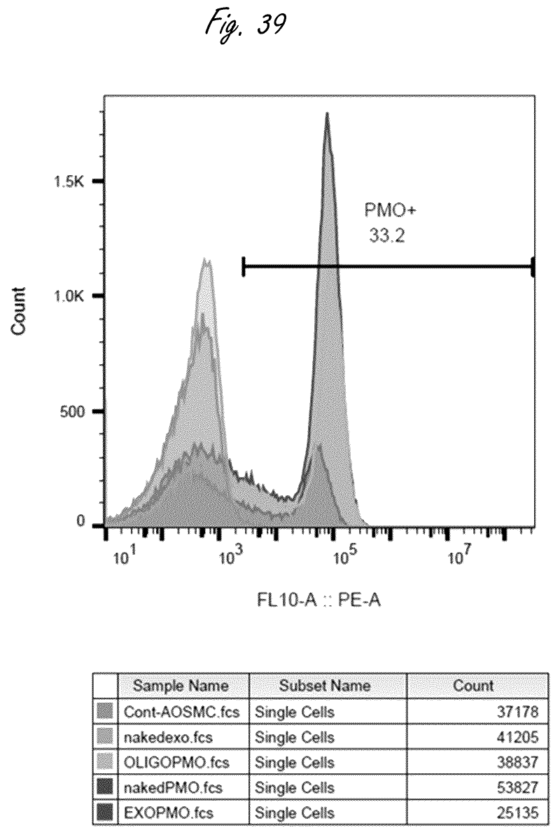

Aortic smooth muscle (AoSM) cells were transfected with 5 pM PMO loaded in exosomes, oligofectamine, or added alone to the cells. After 24 h the cells washed and harvested for flowcytometry. Untreated cells and cells treated with naked exosomes considered as negative control population. Here, it was observed that all PMO ASO 51 transfected cell populations shifted to the right to the PE channel (FIG. 39).

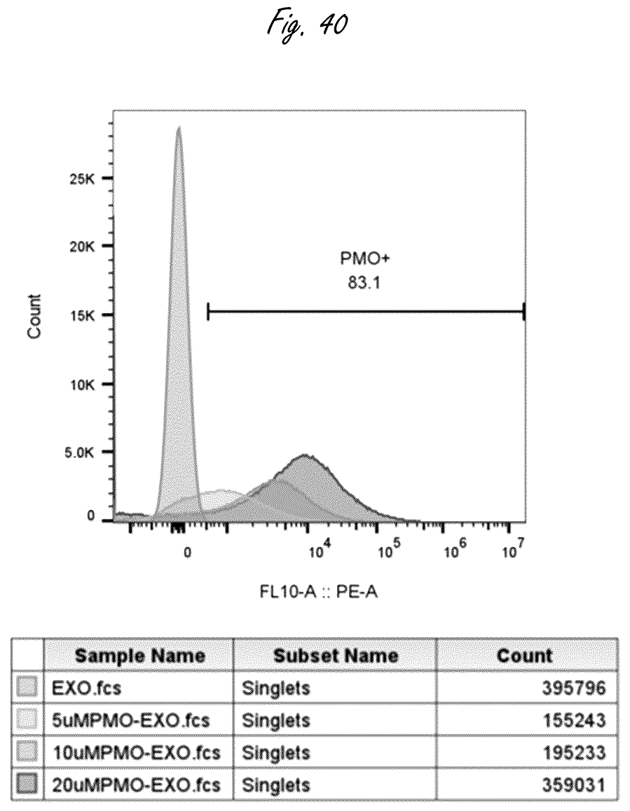

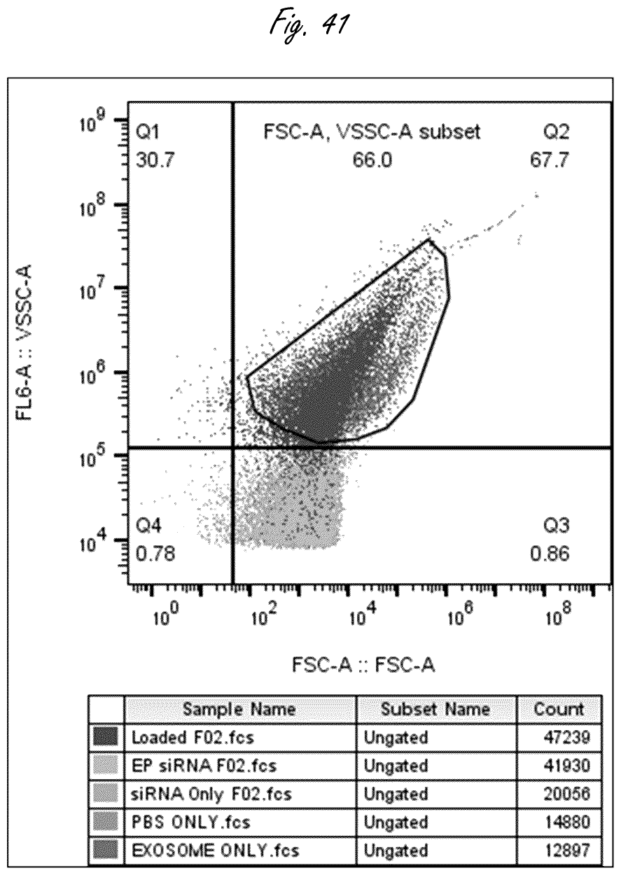

Higher concentrations of PMO ASO 51 were loaded into exosomes (i.e., 5 μM, 10 μM, and 20 μM) using the disclosed heat shock method and with one filtration step. The exosomes were analyzed by flow cytometry and compared to unloaded (“naked”) exosomes, which demonstrated 83.1% exosomes loaded with the PMO ASO 51 (FIG. 40).

### Example 6: mRNA Loading of Exosomes by Heat Shock and Freeze Thaw

Heat shock: A first mixture of 293-derived exosomes, an ARG1 mRNA, and 0.1M Calcium chloride was placed on ice for 40 minutes. Then the mixture was heat shocked by exposure at 42° C. for 60 seconds and then immediately placed on ice for 15 minutes.

Freeze thaw: A second mixture of 293-derived exosomes, an ARG1 mRNA, and 0.1M Calcium chloride was placed on ice for 40 minutes. Then the mixture was frozen at −80° C. for 15 minutes and then immediately thawed at room temperature for 10 minutes. This freeze-thaw cycle was repeated three times.

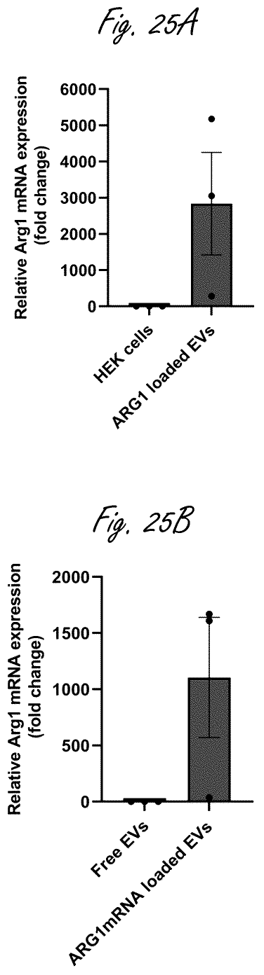

Under both protocols, after heat shock or freeze—that, the loaded exosomes were passed through a desalting matrix (e.g., SpinOUT GT-600 column) for removal of the Calcium chloride and then treated with RNAase A (10 ug/mL) at 37° C. for 30 min to remove siRNAs attached to outside of the exosomes membranes. The total loaded mRNA in exosomes was determined for each method (FIG. 24a) and the loading efficiency determined therefrom (FIG. 24b). Loading efficiency was calculated according to the ARG1 mRNA loaded in EVs to the total ARG1 mRNA used for the reaction (10 ug). Here, for ARG1 mRNA loading, the Calcium chloride heat shock method resulted in higher loading efficiency than the Calcium chloride freeze-thaw method.

More specifically for the heat shock method, a mixture containing 3.2E11 exosome particles, 10 ug mRNA, and 0.1M Calcium chloride was placed on ice for 40 min. Then the mixture was heat shocked at 42° C. for 60 seconds and them immediately placed on ice for 15 minutes. The loaded exosomes mixture was desalted through a SpinOUT GT-600 column and then been treated with RNAase A (10 ug/mL) at 37° C. for 30 minutes to remove mRNAs attached to outside of the exosomes membranes.

Next, HEK293-f-luci cells were seeded in 24 well plates at a concentration of 10E5 cells/well for overnight, to which the ARG1 mRNA-loaded exosomes were added. Controls included: (i) HEK293 exosomes under the Calcium chloride heat shock procedure without loading (FIG. 25A); (ii) HEK293 cells with no exosomes treatment (vehicle control) (FIG. 25B; (ii) HEK293 cells treated with RNAse A; and (iii) ARG1 mRNA treated with RNAse A. After 24 hours of treatment, total RNA was isolated from all transfected cells and vehicle control and quantified by qRT-PCR.

Here, as shown in the right most bars of the histograms in FIGS. 25A-D, cells treated with ARG1 loaded exosomes showed from about 500-fold to about 15,000-fold increase in ARG1 mRNA expression relative to controls (the left most bars of FIGS. 25A-D). Thus, ARG1 mRNA-bearing exosomes produced by calcium heat-shocked cells successfully delivered ARG1 mRNA to recipient cells.

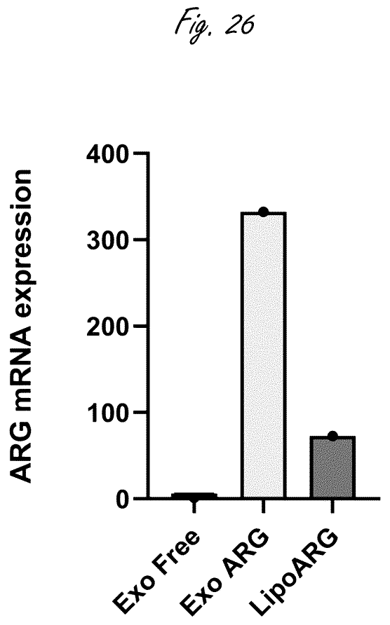

The effectiveness of delivery of mRNA (i.e., e.g., ARG1 mRNA) by way of exosome vehicle delivery relative to delivery by way of lipofectamine was tested. Here, total RNA were isolated from ARG1 Loaded exosomes and then loaded in lipofectamine. HEK293-f-luci cells were seeded in 24 well plates at a concentration of 10E5 cells/well overnight. Then ARG1 loaded lipofectamine or ARG1 mRNA loaded exosomes were used for transfection. After 24 hours, total RNA were isolated from the transfected cells and qPCR run to measure ARG1 mRNA expression. As shown in FIG. 26, about 300% more mRNA was successfully delivered to cells using exosome delivery versus lipofectamine delivery.

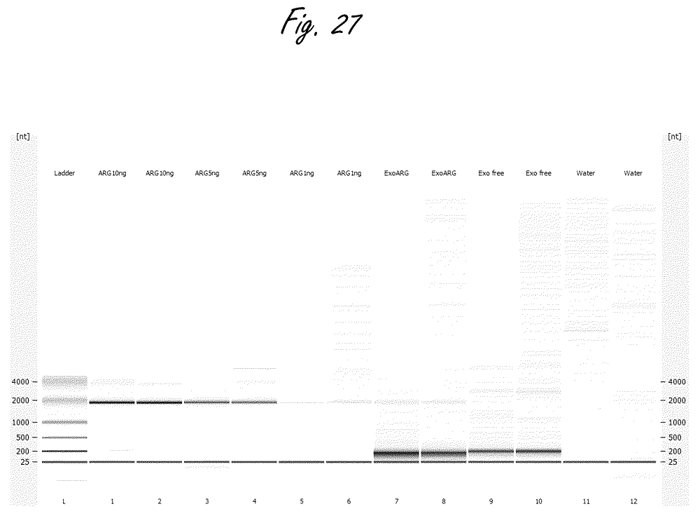

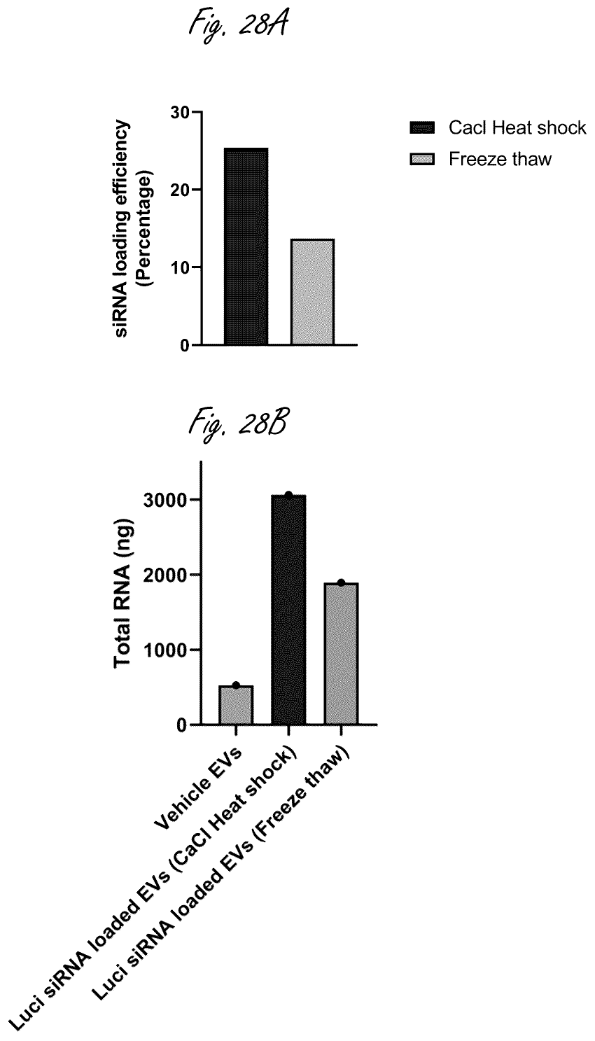

To determine the amount of mRNA (i.e., e.g., ARG1 mRNA) loaded into exosomes, total RNAs from ARG1 mRNA loaded exosomes and total RNAs from free exosomes were isolated and run into bioanalyzer to compare with different concentration of ARG1 mRNA (FIG. 27). Here, the total loaded ARG1 mRNA in 3.2E11 exosome particles was determined to be almost 1 ng.

### Example 7: mRNA Loading of Exosomes by Heat Shock and Freeze Thaw

Calcium chloride heat shock method: A mixture of extracellular vesicles (e.g., exosomes) (3.2E11 particles), siRNA (10 ug), and Calcium chloride (0.1M) was placed on ice for 40 minutes. The mixture was heat shocked at 42° C. for 60 seconds and then immediately placed on ice for additional 15 minutes to recover.

Calcium chloride freeze-thaw method: A mixture of extracellular vesicles (e.g., exosomes) (3.2E11 particles), siRNA (10 ug), and Calcium chloride (0.1M) was placed on ice for 40 minutes. The mixture was then frozen in −80° C. for 15 minutes and then immediately thawed at room temperature for 10 minute. This freeze-thaw cycle was repeated three times.

Here, the exemplar siRNA was designed to interfere with luciferase expression (siRNA-luci).

Following either heat shock or freeze thaw, the loaded exosomes were passed through a SpinOUT GT-600 column for desalting the Calcium chloride followed by treating with RNAase A (10 ug/mL) at 37° C. for 30 minutes to remove siRNAs attached to outside of exosomes membrane.

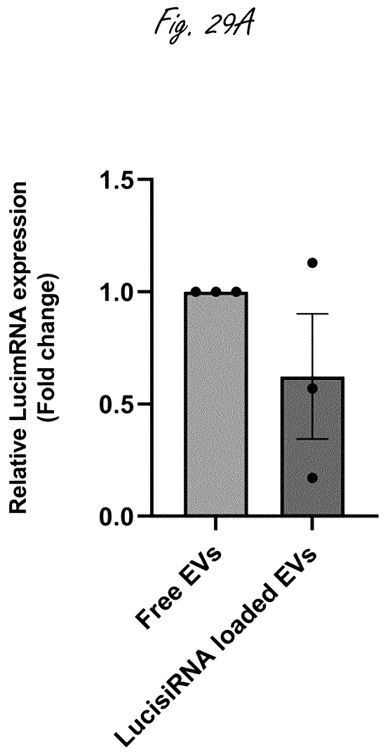

Here, the calculated observed loading efficiency of siRNA-luci into exosomes was observed to be at least about 25% via the Calcium chloride heat shock method and at least about 13% via the Calcium chloride freeze-thaw method (FIGS. 28A and 28B). Loading efficiency was calculated according to the siRNA loaded in the exosomes relative to the total siRNA used for the reaction (in this case, 10 ug).

The effectiveness of siRNA-loaded exosomes to deliver their payload to cells and effect a response was tested. A mixture of EVs (3.2E11 particles), siRNA-luci (10 μg), and Calcium chloride (0.1 M) was placed on ice for 40 minutes, followed by heat shock at 42° C. for 60 seconds, followed by placing on ice for 15 minutes. The siRNA-loaded EVs were passed through a SpinOUT GT-600 column for desalting the Calcium chloride and then treated with RNAase A (10 μg/mL) at 37° C. for 30 min to remove siRNAs attached to the outside of EV (extracellular vesicle) membranes.

The receiving cells, i.e., HEK293-f-luci cells, were seeded in 24 well plates at a concentration of 10E5 cells/well for overnight. Exosomes loaded with the siRNA-luci were added to the treatment wells. After 24 h or 48 h, total RNA was isolated from all transfected receiving cells and the relative expression of luciferase (luci mRNA) in those receiving cells was normalized to GAPDH mRNA expression. Experiments include a negative control (receiving cells with no EV contact) (Exo free), empty exosome control (receiving cells contacted with exosomes that do not contain siRNA-luci) (Free EVs), and experimental group (receiving cells contacted with exosomes containing siRNA-luci) (siRNA Exo).

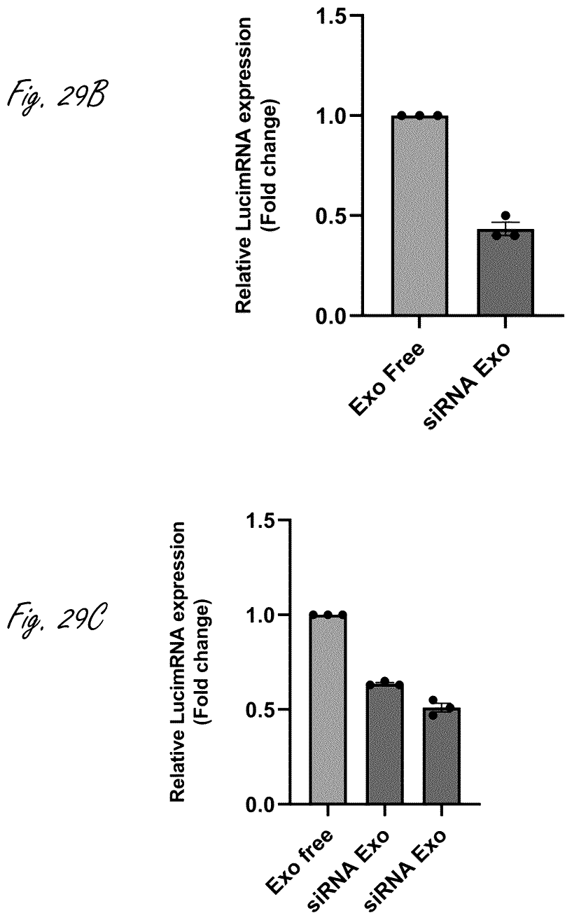

Here, the experimental groups showed reduced luciferase expression in receiving cells of at least about 75% to at least about 50% of control group luciferase expression levels in a dose-dependent manner at 24 hours with 3.2E11 exosomes loaded with 10 μg siRNA-luci [FIG. 29A]; at 48 hours with 3.2E11 exosomes loaded with 10 μg siRNA-luci [FIG. 29B]; and at 48 hours with 3.2E11 or 6.4E11 exosomes loaded with 20 μg siRNA-luci [FIG. 29C].

### Example 8: Exosomes Loaded with siRNA Targeting the RRM2 Gene Trigger Apoptosis in the SKOV3 Ovarian Cancer Cell-Line

Using exosomes as vehicles to deliver therapeutic cargos such as small RNAs has been a novel approach in addressing specific cancers. To load a high concentration of the siRNA therapeutic, exogenous methods of loading have been investigated. Here, it is demonstrated that electroporation as a method of loading yields high loading efficiency with great in vitro functional efficacy.

293F cell derived exosomes were loaded via electroporation, as described herein, with CY3 tagged siRNA targeting the Ribonucleotide Reductase Regulatory Subunit M2 (RRM2) gene. Loading efficiencies (LE) were determined via flow cytometry and RT-qPCR. In vitro cargo delivery (IVCD) was performed on the SKOV3 Ovarian Cancer cell-line. A set number of loaded exosomes loaded with a known concentration of siRNA were added to a set number of SKOV3 cells. Electroporated RRM2 siRNA, and RRM2 siRNA (un-electroporated) were also included as negative controls. Replicate wells (n=3 per method) underwent the following characterizations: cell imaging and viability determination, RRM2 gene expression analysis via RT-qPCR, RRM2 Protein expression analysis via Western blot, and Caspase 3/7 apoptosis assay using a luminometer.

Loading efficiencies of >98% was determined via both flow cytometry and RT-qPCR analyses. IVCD onto the SKOV3 cell line resulted in 70% suppression of the RRM2 gene, resulting in 26% suppression of the RRM2 protein, causing 80% of the cell population to undergo apoptosis. The respective siRNA controls had a negligible effect on the cells.

### Exosome Loading

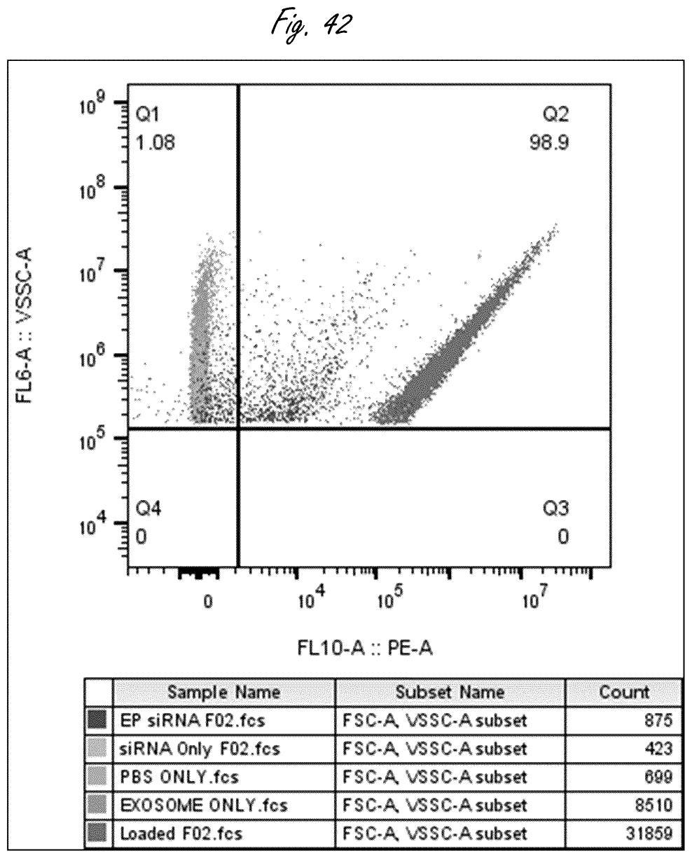

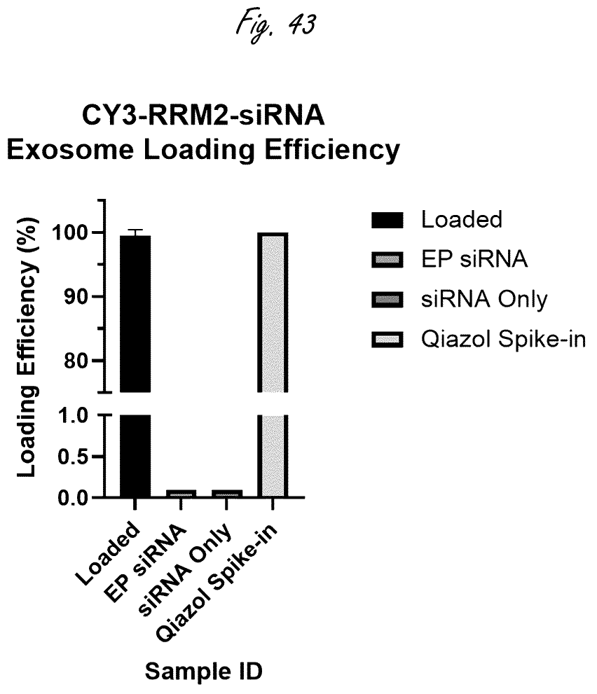

HEK293F cell-derived exosomes were loaded with CY3 tagged siRNA targeting the RRM2 gene. Two separate methods were used to determine loading efficiencies of the siRNA cargo within the exosomes, flow cytometry and RT-qPCR. Flow cytometry analysis exhibiting the ungated population of samples showed the exosome only and siRNA loaded exosomes (Q2), and the PBS, siRNA only and electroporated unloaded (EP) siRNA samples overlap one another (Q3) (FIG. 41). Flow cytometry analysis exhibiting the gated population demonstrated yields >98% loading efficiency of the CY3 tagged siRNA within the loaded exosomes (Q2) (FIG. 42). RT-qPCR analysis of the siRNA-loaded exosomes and respective controls, using the standard curve method for quantification, showed >98% loading efficiency for the siRNA into the exosomes (FIG. 43). The % loading efficiency is used to determine dosing.

### In Vitro Cargo Delivery

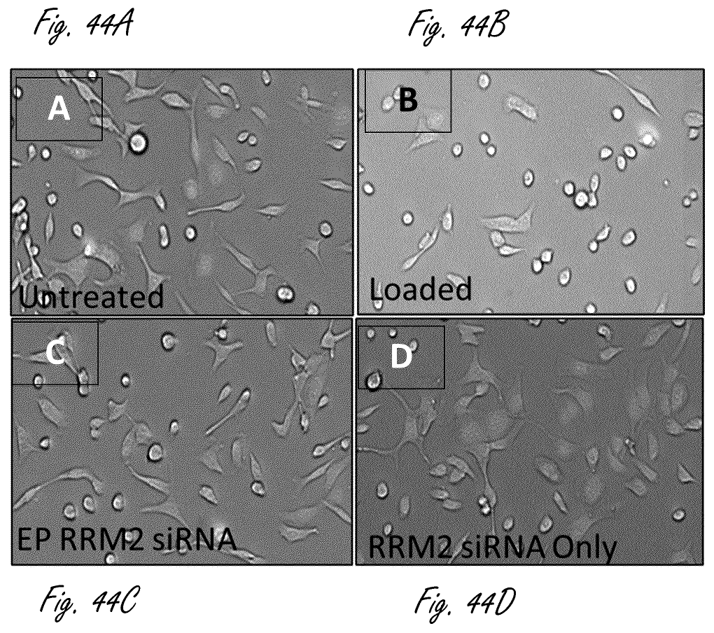

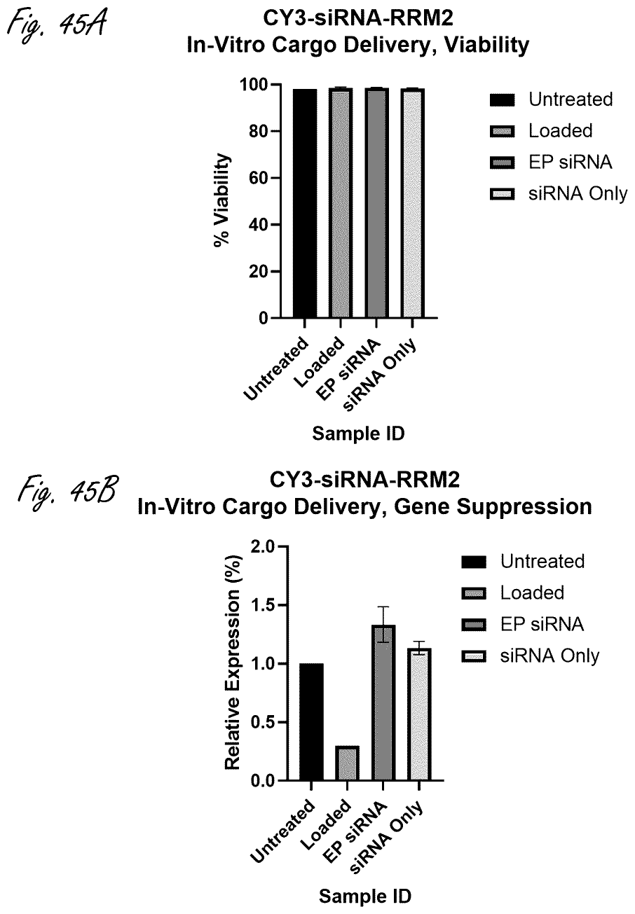

Loaded exosomes and respective controls were added to the SKOV-3 ovarian cancer cell-line at the same dose in triplicate per characterization assay, for treatment overnight. The following day, the SKOV-3 cells were characterized via image analysis, cell viability, and RRM2 gene suppression. For the image analysis and viabilities, cells were stained with Hoechst and propidium iodide (FIG. 44). The wells with the CY3-siRNA loaded exosomes are shown in red color (FIG. 44, panel B) and the wells with the respective controls, i.e., untreated cells (FIG. 44, panel A), are normal (FIG. 44, panels A, C, and D) (Blue: Hoechst nuclear stain, Red: PI dead cell stain). Cell viabilities were >98% post overnight treatment with siRNA-loaded exosomes (controls were untreated cells, cells treated with EP siRNA, and cells treated with siRNA (FIG. 45, panel A). The wells treated with the loaded exosomes were observed to have many rounded cells as they were undergoing apoptosis, their membrane was not yet compromised, and PI-dye was not up-taken within the cells. To measure siRNA effect, RRM2 gene expression was analyzed. Total RNA was isolated from the cells undergoing in vitro cargo delivery for. Post RT-qPCR, double dCT analysis was performed relative to the untreated cells. Only the cells treated with loaded exosomes resulted in 70% RRM2 gene suppression (FIG. 45, panel B).

### Western Blot Analysis

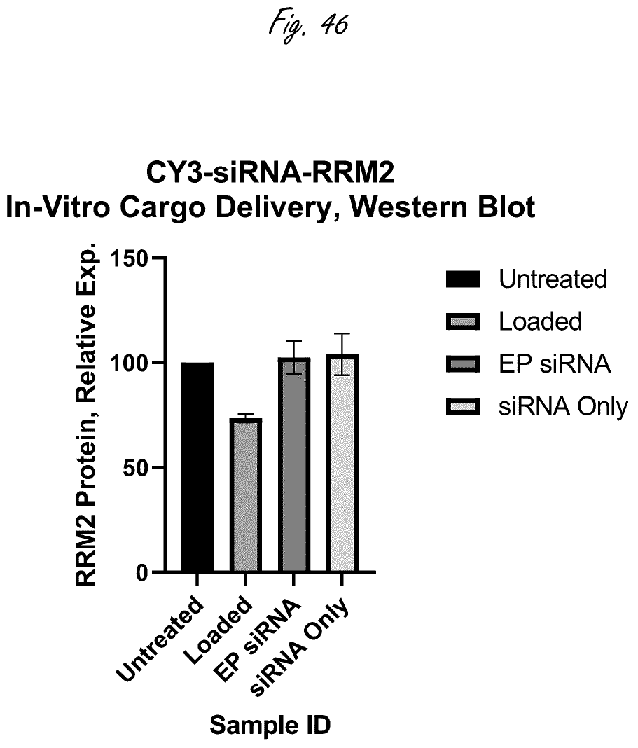

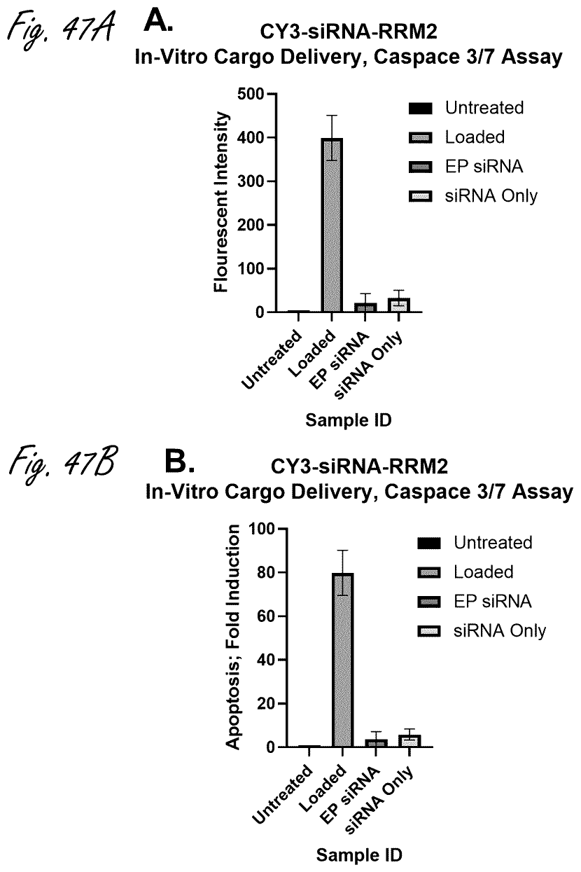

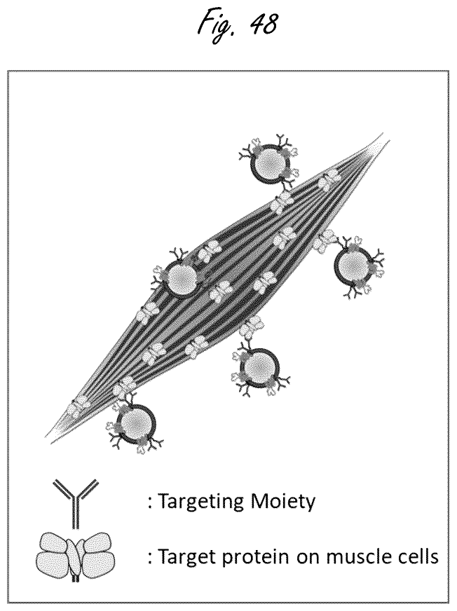

Whole cell protein lysates of SKOV-3 cells treated with RRM2 siRNA-loaded exosomes were analyzed for both b-Actin (˜43 KDa) and RRM2 (˜45 KDa) protein expression levels using the Jess western blot system. The area under the curve generated for both proteins per sample was used to normalize RRM2 protein levels, which in-turn were normalized relative to the untreated control population. The cells treated with loaded exosomes have a 26% reduction in RRM2 protein expression relative to the untreated cells (FIG. 46).

### Apoptosis Assay

A caspase 3/7 fluorescent assay kit was utilized to measure apoptosis (FIG. 47, panel A). The fluorescent intensity of the cells treated with loaded exosomes was used to determine the induction of apoptosis relative to the untreated cell population. Relative to the untreated control population, 80% of the cells treated by the loaded exosomes are undergoing apoptosis (FIG. 47, panel B).

Here, it was demonstrated that 293F cell derived exosomes loaded with CY3-RRM2-siRNA have a potent effect on eradicating the SKOV3 ovarian cancer cell line. Using electroporation to load the exosomes, loading efficiencies of >98% were obtained. Within an in-vitro cargo delivery model, SKOV3 cells treated with the loaded exosomes resulted in 70% reduction in the expression of the RRM2 gene, yielding in 26% reduction in the RRM2 protein which in-turn caused 80% of the cells to undergo apoptosis. Using exosomes loaded with siRNA to target the silencing of the RRM2 gene would be a promising therapeutic/co-therapeutic in combating Ovarian Cancer.

What is claimed is:

1. An engineered vesicle comprising a fusion protein that comprises a first polypeptide sequence and a second polypeptide sequence, wherein:
a. the fusion protein spans a membrane of a vesicle,
b. the first polypeptide sequence comprises a sequence of a targeting moiety,
c. the second polypeptide sequence comprises a sequence of a vesicle protein, and
d. the targeting moiety is positioned outside of the vesicle.

2. The engineered vesicle of claim 1, wherein the targeting moiety comprises a receptor ligand, a receptor, an antibody, a heavy chain only antibody (VHH), a ScFv, a virus antigen, a virus antigen receptor, or fragments or chimeras thereof.

3. The engineered vesicle of claim 1, wherein the targeting moiety comprises a soluble protein, a type I transmembrane protein, or a type II transmembrane protein.

4. The engineered vesicle of claim 1, wherein the targeting moiety binds specifically to no more than five tissues or cell-types.

5. The engineered vesicle of claim 1, wherein the targeting moiety binds to muscle tissue, a muscle cell, or a muscle cell receptor.

6. The engineered vesicle of claim 1, wherein the targeting moiety binds to an acetylcholine receptor, a transferrin receptor, a ryanodine receptor, a cholinergic receptor, a dystrophin, a myosin heavy chain, an alpha actinin, a PRAME family member 9, an FGF8, a protein phosphatase 1 regulatory subunit 27, an isopentenyl-diphosphate delta isomerase 2, a membrane integral NOTCH2 associated receptor 2, a SERCA2, an acetylcholine receptor epsilon, an SCN4A, a muscle specific creatine kinase (CK-MM), or a junctional sarcoplasmic reticulum protein 1.

7. The engineered vesicle of claim 1, wherein the vesicle protein is selected from the group consisting of Lamp-1, Lamp-2, CD13, Flotillin, Syntaxin −3, CD44, ICAM-1, Integrin alpha4, L1CAM, LFA-1, Vti-1A and B, CD9, CD37, CD53, CD63, CD81, CD82, CD151, ICAM-1 and tetraspanins.

8. The engineered vesicle of claim 1, wherein the vesicle is an exosome.

9. The engineered vesicle of claim 1, wherein the vesicle protein is a CD9 protein and the targeting moiety comprises an anti-TfR1 VHH.

10. The engineered vesicle of claim 1, wherein the fusion protein comprises, in order from amino terminus to carboxy terminus, a signal peptide, an ScFv or VHH protein polypeptide, a hinge region, a transmembrane domain polypeptide, a linker polypeptide, and a CD9 polypeptide.

11. The engineered vesicle of claim 1 further comprising a cargo.

12. The engineered vesicle of claim 11, wherein the cargo is a fluorescent dye, a hydrophobic small molecule drug, a hydrophilic small molecule drug, a nucleic acid, a peptide, a peptide amino acid, an antibody or antibody fragment, or a contrast agent.

13. The engineered vesicle of claim 11, wherein the drug cargo is an antisense oligonucleotide (ASO) or a small interfering RNA (siRNA).

14. The engineered vesicle of claim 13, wherein the ASO is an exon-skipping anti-sense oligonucleotide.

15. The engineered vesicle of claim 14, wherein the exon-skipping anti-sense oligonucleotide targets any one or more of exons 2-10 and 45-55 of the human dystrophin gene or murine analog thereof.

16. The engineered vesicle of claim 15, wherein the ASO is a phosphorodiamidate morpholino oligomer.

17. The engineered vesicle of claim 1, wherein the vesicle is derived from a cardiosphere-derived cell (CDC) or a HEK293 cell or derivative thereof.

18. A method of making an engineered vesicle comprising contacting a vesicle that expresses an engineered targeting moiety with a polynucleotide, opening pores in a vesicle membrane, allowing the polynucleotide to enter the vesicle, and closing the pores, wherein the polynucleotide is contained within the vesicle.

19. The method of claim 18, wherein the pores are opened in the vesicle membrane by heat shocking the vesicle, by freeze-thawing the vesicle, by sonicating the vesicle, or by subjecting the vesicle to a voltage potential.

20. A method of treating a muscle disease comprising administering an engineered vesicle of claim 1 to a subject in need thereof.

21. The method of claim 20, wherein the muscle disease is a muscular dystrophy.


SEQUENCE LISTING (13 sequences)

SEQ ID NO: 1 - protein, 236 aa, synthetic construct
QLVESGGGVVQPGGSLRLSCAASGEIFSINFMRWYRQAPGKQREWVAGFTRDGSTNYPDSAKGRFTISRDNAKNTVYLQIDSLKPEDTAVYYCYMLDTWGQGTQVTVSSGSTSGSGKPGSGEGSTKGEVQLVESGGGVVQPGGSLRLSCAASGEIFSINFMRWYRQAPGKQREWVAGFTRDGSTNYPDSAKGRFTISRDNAKNTVYLQIDSLKPEDTAVYYCYMLDTWGQGTQVTV

SEQ ID NO: 2 - protein, 579 aa, synthetic construct
MLLLVTSLLLCELPHPAFLLIPEVQLVESGGGVVQPGGSLRLSCAASGEIFSINFMRWYRQAPGKQREWVAGFTRDGSTNYPDSAKGRFTISRDNAKNTVYLQIDSLKPEDTAVYYCYMLDTWGQGTQVTVSSGSTSGSGKPGSGEGSTKGEVQLVESGGGVVQPGGSLRLSCAASGEIFSINFMRWYRQAPGKQREWVAGFTRDGSTNYPDSAKGRFTISRDNAKNTVYLQIDSLKPEDTAVYYCYMLDTWGQGTQVTVSSKPTTTPAPRPPTPAPTIASQPLSLRPEACRPAAGGAVHTRGLDFACDIYIWAPLAGTCGVLLLSLVITLYCNHRNGGGGSGGGGSGGGGSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 3 - DNA, 1749 bp, synthetic construct
  source 1..1749: mol_type = other DNA
gccaccatgctgctgctggtgaccagcctgctgctgtgtgagctgccccaccccgcctttctgctgatccccgaggtgcagctggtggagtctgggggaggcgtggtgcagcctggggggtctctgagactctcctgtgcagcctctggagagatcttcagtatcaattttatgcgctggtaccgccaggctccagggaagcagcgcgagtgggtcgcaggttttactagggatggaagcacaaactatccagactccgcgaagggccgattcaccatctctagagacaacgccaagaacacggtgtatctgcaaatagacagcctgaaacctgaggacacggccgtctattattgttatatgttggacacctggggccaggggacccaggtcactgtctcctcaggctccacctctggcagcggcaagccaggcagcggagagggctccacaaagggagaggtgcagctggtggagtctgggggaggcgtggtgcagcctggggggtctctgagactctcctgtgcagcctctggagagatcttcagtatcaattttatgcgctggtaccgccaggctccagggaagcagcgcgagtgggtcgcaggttttactagggatggaagcacaaactatccagactccgcgaagggccgattcaccatctctagagacaacgccaagaacacggtgtatctgcaaatagacagcctgaaacctgaggacacggccgtctattattgttatatgttggacacctggggccaggggacccaggtcactgtctcctcaaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaatggcggaggcggaagcggaggcggaggctccggcggaggcggaagcccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctaataa

SEQ ID NO: 4 - DNA, 1749 bp, synthetic construct; identical to SEQ ID NO: 3
  source 1..1749: mol_type = other DNA

SEQ ID NO: 5 - protein, 236 aa, synthetic construct
QLVESGGGEVQPGGSLKLSCVASGTDFSINFVRWYRQRPGKQREWVAGFTANGDTNYPDSMKGRFTISRDNAKNTVYLQINSLKSEDTAVYYCYMLDNWGQGTQVTVSSGSTSGSGKPGSGEGSTKGEVQLVESGGGEVQPGGSLKLSCVASGTDFSINFVRWYRQRPGKQREWVAGFTANGDTNYPDSMKGRFTISRDNAKNTVYLQINSLKSEDTAVYYCYMLDNWGQGTQVTV

SEQ ID NO: 6 - protein, 579 aa, synthetic construct
MLLLVTSLLLCELPHPAFLLIPEVQLVESGGGEVQPGGSLKLSCVASGTDFSINFVRWYRQRPGKQREWVAGFTANGDTNYPDSMKGRFTISRDNAKNTVYLQINSLKSEDTAVYYCYMLDNWGQGTQVTVSSGSTSGSGKPGSGEGSTKGEVQLVESGGGEVQPGGSLKLSCVASGTDFSINFVRWYRQRPGKQREWVAGFTANGDTNYPDSMKGRFTISRDNAKNTVYLQINSLKSEDTAVYYCYMLDNWGQGTQVTVSSKPTTTPAPRPPTPAPTIASQPLSLRPEACRPAAGGAVHTRGLDFACDIYIWAPLAGTCGVLLLSLVITLYCNHRNGGGGSGGGGSGGGGSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 7 - DNA, 1749 bp, synthetic construct
  source 1..1749: mol_type = other DNA
gccaccatgctgctgctggtgaccagcctgctgctgtgtgagctgccccaccccgcctttctgctgatccccgaggtgcagctggtggagtctgggggaggcgaggtgcagcctggggggtctctgaaactctcctgtgtagcctctggaaccgacttcagtatcaattttgtgcgctggtaccgtcagcgtccagggaagcagcgcgagtgggtcgcaggatttactgcgaatggtgatacaaactatccagactccatgaaggggcgattcaccatttccagagacaacgccaagaatacggtgtatctacagataaacagcctgaaatctgaggacacggccgtctattattgctatatgttagataattggggccaggggacccaggtcaccgtctcctcaggctccacctctggcagcggcaagccaggcagcggagagggctccacaaagggagaggtgcagctggtggagtctgggggaggcgaggtgcagcctggggggtctctgaaactctcctgtgtagcctctggaaccgacttcagtatcaattttgtgcgctggtaccgtcagcgtccagggaagcagcgcgagtgggtcgcaggatttactgcgaatggtgatacaaactatccagactccatgaaggggcgattcaccatttccagagacaacgccaagaatacggtgtatctacagataaacagcctgaaatctgaggacacggccgtctattattgctatatgttagataattggggccaggggacccaggtcaccgtctcctcaaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaatggcggaggcggaagcggaggcggaggctccggcggaggcggaagcccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctaataa

SEQ ID NO: 8 - DNA, 9390 bp, synthetic construct
  source 1..9390: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccggcgcgccagatctcaagcttaactagctagcgccaccatgctgctgctggtgaccagcctgctgctgtgtgagctgccccaccccgcctttctgctgatccccgaggtgcagctggtggagtctgggggaggcgaggtgcagcctggggggtctctgaaactctcctgtgtagcctctggaaccgacttcagtatcaattttgtgcgctggtaccgtcagcgtccagggaagcagcgcgagtgggtcgcaggatttactgcgaatggtgatacaaactatccagactccatgaaggggcgattcaccatttccagagacaacgccaagaatacggtgtatctacagataaacagcctgaaatctgaggacacggccgtctattattgctatatgttagataattggggccaggggacccaggtcaccgtctcctcaggctccacctctggcagcggcaagccaggcagcggagagggctccacaaagggagaggtgcagctggtggagtctgggggaggcgaggtgcagcctggggggtctctgaaactctcctgtgtagcctctggaaccgacttcagtatcaattttgtgcgctggtaccgtcagcgtccagggaagcagcgcgagtgggtcgcaggatttactgcgaatggtgatacaaactatccagactccatgaaggggcgattcaccatttccagagacaacgccaagaatacggtgtatctacagataaacagcctgaaatctgaggacacggccgtctattattgctatatgttagataattggggccaggggacccaggtcaccgtctcctcaaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaatggcggaggcggaagcggaggcggaggctccggcggaggcggaagcccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctaataagtttaaacctaggcgtagcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 9 - RNA, 21 nt, synthetic construct
  source 1..21: mol_type = other RNA
ggagtgatgtcaagtccaatt

SEQ ID NO: 10 - RNA, 21 nt, synthetic construct
  source 1..21: mol_type = other RNA
ttggacttgacatcactcctt

SEQ ID NO: 11 - DNA, 21 bp, synthetic construct
  source 1..21: mol_type = other DNA
gcgaagaaggagaatagggtt

SEQ ID NO: 12 - RNA, 25 nt, synthetic construct
  source 1..25: mol_type = other RNA
gaacacctcagaaccggaggcaact

SEQ ID NO: 13 - DNA, 28 bp, synthetic construct
  source 1..28: mol_type = other DNA
ctccaacatcaaggaagatggcatttct